SEQUENCE LISTING (45 sequences)

SEQ ID NO: 1 - DNA, 1301 bp, Artificial sequence
  cDNA of Atlg09350
acttatcaacaactcaaaactgatcatcacaaagaaaagagttttctctgataatctttctcggacaagatggcacctgagatgaacaacaagttgagctacggagaaaagaagagagcgtacgttacgttcctcgccggaaccggagactacgtgaaaggagtggttggtctggctaaagggctaaggaaaactaaaagcaagtacccattagtggttgctgtgttacctgacgtgccggccgatcaccggagacagctattggaccaaggctgcgtcatcaaggagattcagccggtttacccaccggataaccaaactcagtttgctatggcttactacgtcctcaactactctaaacttcgtatttggaagtttgtagagtacagcaagctgatatacttagacggagacatacaagtgtttgagaacatagatcacttgtttgatcttcctgacggcaacttctacgccgttaaagactgtttctgcgagaagacttggagccacacgcctcagtacaagattggctactgccaacagtgtccggacaaggtgacgtggcctgagtcagagcttggtcctaagccaccgttgtacttcaacgccggcatgttcgtctacgagccaagcctccccacttattacaaccttttggagacactcaaagttgtccctcccacaccttttgctgaacaggatttcttgaacatgtacttcaaagacatatacaagcctattccaccagtttacaatcttgtcttggccatgctctggaggcatccagagaacatagagcttaacgaagctaaggttgttcattactgtgcagccggtgctaagccttggaggttcacaggccaagaaggaaatatggagagggaagacatcaagatgcttgtagagaaatggtgggacatttacaacgacgagtctcttgactacaaaaactttaatgtgcattgcggacaaaaagaagatgttcacaggaaaccgaaaacccttccacagttctttacagatttgtctgaagctgatgtgcttcaatgtgccaaagctccatccgcggcttagacaactttgtgagggctgacgagcgaggttcttgtcaacaccagtttgctaatagtttgtgaaactggtgatgtttacttaggaatttgttatatgaaattgtgctttactgttttttaatttactgaattaagaggactttttaatccccaaaccctctttgtaccttatatgtttgctatggactttccttcctaaaatacaatgaaaatatatatagttttgtt

SEQ ID NO: 2 - protein, 334 aa, Arabidopsis thaliana
  protein sequence of Atlg09350
MAPEMNNKLSYGEKKRAYVTFLAGTGDYVKGVVGLAKGLRKTKSKYPLVVAVLPDVPADHRRQLLDQGCVIKEIQPVYPPDNQTQFAMAYYVLNYSKLRIWKFVEYSKLIYLDGDIQVFENIDHLFDLPDGNFYAVKDCFCEKTWSHTPQYKIGYCQQCPDKVTWPESELGPKPPLYFNAGMFVYEPSLPTYYNLLETLKVVPPTPFAEQDFLNMYFKDIYKPIPPVYNLVLAMLWRHPENIELNEAKVVHYCAAGAKPWRFTGQEGNMEREDIKMLVEKWWDIYNDESLDYKNFNVHCGQKEDVHRKPKTLPQFFTDLSEADVLQCAKAPSAA

SEQ ID NO: 3 - DNA, 1623 bp, Arabidopsis thaliana
  Genomic sequence lg09350
acttatcaacaactcaaaactgatcatcacaaagaaaagagttttctctgataatctttctcggacaagatggcacctgagatgaacaacaagttgagctacggagaaaagaagagagcgtacgttacgttcctcgccggaaccggagactacgtgaaaggagtggttggtctggctaaagggctaaggaaaactaaaagcaagtacccattagtggttgctgtgttacctgacgtgccggccgatcaccggagacagctattggaccaaggctgcgtcatcaaggagattcagccggtttacccaccggataaccaaactcagtttgctatggcttactacgtcctcaactactctaaacttcgtatttggaaggtatttatatacatgatagtatctaggtttaaaagacttgagaatctgaatttaagttttcttgatgaaacaataactatttggatgcagtttgtagagtacagcaagctgatatacttagacggagacatacaagtgtttgagaacatagatcacttgtttgatcttcctgacggcaacttctacgccgttaaagactgtttctgcgagaagacttggagccacacgcctcagtacaagattggctactgccaacagtgtccggacaaggtgacgtggcctgagtcagagcttggtcctaagccaccgttgtacttcaacgccggcatgttcgtctacgagccaagcctccccacttattacaaccttttggagacactcaaagttgtccctcccacaccttttgctgaacaggtaatacaaaatctacacagtatatatatatatatatatatatatatatatatgattatatacatacatggggcagctaccactatacatattagaggtcttgtaccttactctaatattatgaggctaagttgatttgtgtgtgttttataggatttcttgaacatgtacttcaaagacatatacaagcctattccaccagtttacaatcttgtcttggccatgctctggaggcatccagagaacatagagcttaacgaagctaaggttgttcattactgtgcagccggtgctaagccttggaggtatgatctaattatcttcttgttaatttaaaaaaaaaaaggtgaaagatctgaaattgcttctctgttttgtgaataggttcacaggccaagaaggaaatatggagagggaagacatcaagatgcttgtagagaaatggtgggacatttacaacgacgagtctcttgactacaaaaactttaatgtgcattgcggacaaaaagaagatgttcacaggaaaccgaaaacccttccacagttctttacagatttgtctgaagctgatgtgcttcaatgtgccaaagctccatccgcggcttagacaactttgtgagggctgacgagcgaggttcttgtcaacaccagtttgctaatagtttgtgaaactggtgatgtttacttaggaatttgttatatgaaattgtgctttactgttttttaatttactgaattaagaggactttttaatccccaaaccctctttgtaccttatatgtttgctatggactttccttcctaaaatacaatgaaaatatatatagttttgtt

SEQ ID NO: 4 - protein, 335 aa, Arabidopsis thaliana
  GolS2 protein sequence
MAPEINTKLTVPVHSATGGEKRAYVTFLAGTGDYVKGVVGLAKGLRKAKSKYPLVVAVLPDVPEDHRKQLVDQGCVVKEIEPVYPPENQTEFAMAYYVINYSKLRIWEFVEYNKMIYLDGDIQVFDNIDHLFDLPNGQFYAVMDCFCEKTWSHSPQYKIGYCQQCPDKVTWPEAKLGPKPPLYFNAGMFVYEPNLSTYHNLLETVKIVPPTLFAEQDFLNMYFKDIYKPIPPVYNLVLAMLWRHPENIELDQVKVVHYCAAGAKPWRFTGEEENMDREDIKMLVKKWWDIYNDESLDYKNVVIGDSHKKQQTLQQFIEALSEAGALQYVKAPSAA

SEQ ID NO: 5 - DNA, 1396 bp, Arabidopsis thaliana
  GolS2 nucleotide sequence
actcagcacactacacacaaactaaacgaaacaaaaacaaacacaaacacaaacacagaagtattttctaaaagacaaagaagatggcacctgagatcaataccaaactcaccgtaccggtccactcagcaaccggcggagaaaagagggcgtacgtgactttccttgctggaactggagactacgtgaaaggggtggtgggtcttgctaaggggctaaggaaagctaagagcaagtacccactcgtggtggcggttttgccggatgtaccggaggatcaccggaaacagttggtggatcaaggctgtgttgttaaggagatagagccggtttatccaccggagaatcaaaccgagtttgctatggcttactatgtcattaactactctaagcttcgtatctgggagttcgtggagtacaacaagatgatatacttggacggtgacatacaagtgttcgacaacatagatcacttgtttgatcttccaaacggccaattctatgcagttatggactgtttttgcgagaagacatggagtcactcgccacagtacaagatcggttattgccaacagtgtccggacaaggtgacatggccagaggccaagcttggtcctaagccgcctttgtacttcaacgccggtatgttcgtttacgagcctaacctctccacttatcataacctcttggagacggtcaaaattgtgcctcccactctttttgctgaacaggattttttgaatatgtacttcaaggacatatacaagccaataccaccagtgtataacttggtcttggcaatgctttggaggcatccagagaatatagagcttgaccaagtcaaagttgttcactactgtgctgctggtgctaaaccttggagattcactggagaagaagagaacatggacagagaagacattaagatgctggtcaagaaatggtgggacatttacaatgacgagtctcttgattacaagaatgttgtcattggggacagccacaagaagcaacagacacttcagcagtttatcgaagctttgtctgaagccggtgcgcttcagtatgtcaaagctccatctgcagcttagccacaatataatcatcgagctcgttgctgcatacgcctcttcatgactataatattgcttattgctttagtttgaaacttgtttataggaccatatagttatacatatactatatgagattgtgttactattttaatttttatgggttttggaggattttttaaatttccacatccgtttttacttatcatatactagcttgttgattaggaacaatgctcccgacaatattgtaaagtatacttgtttaagttgttatccatgagaatacaaacgtatatagttcagttatatttatattcttc

SEQ ID NO: 6 - protein, 344 aa, Arabidopsis thaliana
  GolS1 protein sequence
MAPGLTQTADAMSTVTITKPSLPSVQDSDRAYVTFLAGNGDYVKGVVGLAKGLRKVKSAYPLVVAMLPDVPEEHRRILVDQGCIVREIEPVYPPENQTQFAMAYYVINYSKLRIWKFVEYSKMIYLDGDIQVYENIDHLFDLPDGYLYAVMDCFCEKTWSHTPQYKIRYCQQCPDKVQWPKAELGEPPALYFNAGMFLYEPNLETYEDLLRTLKITPPTPFAEQDFLNMYFKKIYKPIPLVYNLVLAMLWRHPENVELGKVKVVHYCAAGSKPWRYTGKEANMEREDIKMLVKKWWDIYDDESLDYKKPVTVVDTEVDLVNLKPFITALTEAGRLNYVTAPSAA

SEQ ID NO: 7 - DNA, 1355 bp, Arabidopsis thaliana
  GolS1 nucleotide sequence
ctcatcagcaaacacaaacacacgactagataaactctaaatcctcacagcagatcacgtgctaatcacatggctccggggcttactcaaaccgctgatgctatgtccaccgtgacgataacaaaaccgtcactgccatcagtccaagacagcgatcgagcttacgtgacgtttcttgctggaaacggtgattacgtgaaaggagtcgttggtttagccaaagggttaaggaaagtcaaatcggcttatccactcgtagtagcgatgttacccgacgtcccggaggaacaccgtcgtatacttgtggatcaaggatgcatcgtccgtgaaatcgaacccgtttacccacccgagaaccaaactcagttcgccatggcttattacgtcatcaactactctaaactccgtatctggaagtttgtggagtatagtaaaatgatatatttagatggagacattcaagtttacgaaaacatcgatcacttgtttgacctaccagatggctatttgtacgcggtgatggattgtttctgtgagaaaacatggagtcacacgccgcaatacaagatcagatattgccaacaatgccccgacaaagtccagtggccaaaagcggagcttggagagccaccggctctttacttcaacgccggaatgttcttgtacgagcctaacctcgagacttacgaggatctactacgaacacttaaaatcactcctccgactcctttcgctgaacaggattttttgaacatgtactttaagaaaatctacaagccgattcctttagtgtacaatctcgtccttgcgatgttatggcgtcacccagaaaatgtagagcttggaaaagtcaaggtggttcactactgtgcagcgggttcgaagccgtggagatacacagggaaagaagcgaacatggagagggaagatataaaaatgttagtgaaaaaatggtgggacatttacgacgacgaatccttggattacaagaaacctgttaccgttgtggacacagaggtcgatctcgtgaatctgaagccgttcatcaccgctcttactgaagctggccggctcaactacgtgaccgcaccgtccgctgcttgaatgttgccaggagttaaaattgtcggtggatcaggtagttatttttctttccggtcagtaagaggttggggattatgtgtcgggtcgacaaccacacatactgtctctggttccaatgctttggacgttgacatgtgatttgctttacttttttgtctataatgtgtgttatcctcaattttagcgtctatcttcgaatatcaaacttgagtgacaaaataaatattccaaataacaggtcttcttcacac

SEQ ID NO: 8 - protein, 328 aa, Glycine max
  GolS protein sequence
MAPNITTVKTTITDAQAKVATDHGRAYVTFLAGNGDYVKGVVGLAKGLRKVKSMYPLVVAVLPDVPQDHRNILTSQGCIVREIEPVYPPENQTQFAMAYYVINYSKLRIWEFVEYSKMIYLDGDIQVFDNIDHLFDLPDNYFYAVMDCFCEPTWGHTKQYQIGYCQQCPHKVQWPTHFGPKPPLYFNAGMFVYEPNLATYRDLLQTVQVTQPTSFAEQDFLNIYFKDKYRPIPNVYNLVLAMLWRHPENVELDKVKVVHYCAAGSKPWRYTGKEENMEREDIKMLVKKWWDIYEDETLDYNNPLNVDKFTAALMEVGEVKFVRAPSAA

SEQ ID NO: 9 - protein, 40 aa, Cucurbita pepo
  GolS protein sequence
MSPAATETAIESTDAPKRAFVTFLAGNGDYWKGVVGLAKG

SEQ ID NO: 10 - DNA, 195 bp, Artificial sequence
  portion of Atlg09350 nucleotide sequence
tatttggatgcagtttgtagagtacagcaagctgatatacttagacggagacatacaagtgtttgagaacatagatcacttgtttgatcttcctgacggcaacttctacgccgttaaagactgtttctgcgagaagacttggagccacacgcctcagtacaagattggctactgccaacagtgtccggacaaggt

SEQ ID NO: 11 - DNA, 193 bp, Artificial sequence
  portion of BT097874.1 nucleotide sequence
tatttgggagttcgtggagtacaagaagacgatatacctagacggtgacatccaagtatttggaaacatagaccacttgtttgatctgcctgataattatttctatgcggtgatggattgtttctgcgagaagacttggagccacaccccctcagttccagattgggtactgccaacagtgccctgataaggt

SEQ ID NO: 12 - DNA, 1406 bp, Glycine max
  GolS nucleotide sequence
agccaaaagtttgttttcatagtgtgttttgtttcccaaatcctactcttgtgaccacaacccttcctcctctttcttttgaaacctctttttttctattccccaaccaaacaagcaaacgctactcactcatcatcactgagatcatggctcctaatatcaccactgtcaaaaccaccatcaccgacgctcaagccaaggtcgccaccgatcatggtcgtgcctacgtcaccttcctcgccggaaacggtgactatgtgaaaggtgtcgttggcttggcaaaaggtctgagaaaagtgaagagcatgtaccctctggtggttgcagtgctacccgatgttccccaagatcaccgcaacattctcacctcccaaggttgcattgttagagagattgagcccgtgtaccccccagagaatcaaacccagtttgccatggcatattacgtcatcaactattccaagctacgtatttgggagtttgtggagtacagcaagatgatatacctagacggtgatatccaagtttttgacaacattgaccacttgtttgacttgcctgataactacttctatgcggtgatggactgtttctgtgagccaacttggggccacactaaacaatatcagatcggttactgccagcagtgcccccataaggttcagtggcccactcactttgggcccaaacctcctctctatttcaatgctggcatgtttgtgtatgagcccaatttggctacttaccgtgacctccttcaaacagtccaagtcacccagcccacttcctttgctgaacaggattttttgaacatttacttcaaggacaaatataggccaattcctaatgtctacaatcttgtgctggccatgctgtggcgtcaccctgagaacgttgagcttgacaaagttaaagtggttcactactgtgctgctgggtctaagccttggaggtacactgggaaggaggagaatatggagagagaagatatcaagatgttagtgaaaaagtggtgggatatatatgaggatgagactttggactacaacaatccactcaatgtggataagttcactgcggcacttatggaggttggtgaagtcaagttcgtccgtgccccatctgctgcttaagagtgtctttggaaatcaagtgtgatccaagtacatgtacaaagtcatacatcattacattaacttttatgtatttctaaaagtcatacatcattacattaagttttatgtatttctaaagtcttaagacttaagaggaccttttttatgtgtcccggcttttctttttttctttttccaattctgtcattgtaaagcaggtgaataccggtatccttaattttataaatggatatgaattttattttgcaaaaaaaaaaaaaaaaaaaaaaa

SEQ ID NO: 13 - DNA, 1807 bp, Cucurbita pepo
  GAS1 gene
actgttcaaggtcaaagataaagttctctagagttttggtatcttgagccccagagaagggcttgggctccaagacctttatcttgttgaattagactacaccccagtcggggtttggttccccaccgttcttatggtgagatttaatctagcgctaagatttgcgacttcaaccctaaccaccactccgacgacttctcttgaatcctcaaacatttctgagaccatctgaacgatcgcttgttgggcattgtctaagcgttcattgcgttcttcgattcgggcaacagaggccatcgagctagccccaagctcgaagccaccagcagtagtggccttgtcttctaaggtctcaacagtaaccattaattcacggacatacattacctcaaggtggcctaccatcttgtcgatcctataggctttagcggcaatctcattcatgcaagattccaaaaaccacattccatcaggtcagtcttgtcgagttgttttgcaatgggacatatttgtttactacaattcaaggagtcgactctgctctaataccacttatcacaatcacactctcgtggcattgggacaacgattgtgcagcacttgttccacatgaaaaggggctagattctaacctgagttttatgaaaaagggcgagattctaacctgagttttaaacaccttcttggctttcctatatccatcttaatctataggtacccaagttggtgtaatgaagaataatctgaaaatgatttgagttctttggattataagaacagaaattgttttagttcttatttgatgatgtttagaaggcatgaaatgagttgggaaaaagtgcagaatcctataaaaagttggaaataacagccttcacaccaatggaatactgaggtgcactagaagacaacgtatttccacaaagctaagctgattcaaccgtgtggggcatcttcttcttcaaaaccattcaaattgtatatacaagaatcgaatcatctggacaaaagcagctttccttttcttttcattctttttttccactttgtgtagtgcctaagtctctacgcctcgtgtccaccccttcaggactcagcctcctcgcgggcacatcactcagtgtctggctctgataccatttataacgctccaactccactgctagccgagattgtcctctttggactttctcttcaaagtttttaaaacgcgtctagtaggaagaggtttctatacccttacatttatacgagcatagaatcgtaatatattaaataaaatgtcaaagtctgagattcttagatcctccccaaattgtccacatttgtcttgtctacaaacttgcaagagagggaatgatccataattaagatgagtatccaaaaggcaaaggggatgatccaacggtgcaaaacataagaatcttgtccatctcacggcttaaggataggattccaactcatattcatcccaccaatcccattctcttaattgcagtgtttgtattaataccaagtaatcacaatactaacatgctcccccacggctttataaatacctgcattcccatccctccacaactcaagcttgaaggactaaacctacaacttaaacctgccctacaagtccttcttgcctccctttctcaacaactcttttgttgttccacccacataatcaatatgtctccggctgccaccgaaactgccatcgagtcgaccgatgcccccaagagggcattcgtgacgttcttggccggtaatggagactattggaaaggtgtagttggattggcaaagggtctca

SEQ ID NO: 14 - DNA, 36 bp, Artificial sequence
  Primer AtGolS3.Fw 5′
cgcggatccatggcacctgagatgaacaacaagttg

SEQ ID NO: 15 - DNA, 33 bp, Artificial sequence
  Primer AtGolS3.Rv 5′
cgcgagctcctggtgttgacaagaacctcgctc

SEQ ID NO: 16 - protein, 334 aa, Populus Trichocarpa
  GolS3 protein sequence
MAPDITTPLANNATTLVKQASISSCAYVTFLAGDGDYWKGVVGLAKGLRKAESKYPLVVAILPDVPEEHRKILVSQGCIVREIEPVHPPENQTRFAMPYYVINYSKLRIWEFVEYSKMIYLDGDIQVFDNIDHLFDMPDGCFYAVMDCFCEKTWSNSPQYKIGYCQQCPDKVQWPAEMGPKPPLYFNAGMFVYEPNLSTYHDLLETLKITSPTLFAEQDFLNMFFRDVYKPIPSDYNLVLAMLWRHPENINLDKVKVVHYCAAGSKPWRYTGKEENMDREDIKMLVQKWWDIYNDESLDHKNTVVASSGSELQPILEALYEAGVDLHFTAPSAA

SEQ ID NO: 17 - protein, 334 aa, Populus Trichocarpa
  GolS5 protein sequence
MAPDITATLANNTNSLVKQASISSCAYVTFLAGDGDYWKGVVGLAKGLRKAKCNYPLVVAILPDVPEEHRKILASQGCIVREIEPVNPPENQTQFAMAYYVINYSKLRIWEFVEYSKMIYLDGDIQVFDSIDHLFDMPDGYFYAAMDCFCEKTWSNSPQYKIGYCQQCPDKVHWPAEMGPKPPLYFNAGMFVYEPNLSTYHDLLETLKVTPPTLFAEQDFLNMFFRDVYKPIPSDYNLVLALLWRHPENINLDKVKVVHYCAAGSKPWRYTGKEDNMDREDIKMLVNKWWDIYHDESLDYKNTVVAAAGAELQPFLAALSEAGIAHYITAPSAA

SEQ ID NO: 18 - protein, 336 aa, Populus Trichocarpa
  GolS7 protein sequence
MAPELVQAALKPAGFTKPASLPSRAYVTFLAGNGDYVKGVVGLAKGLRKVKTAYPLIVAVLPDVPEEHRRILESQGCIVREIEPVYPPENQTQFAMAYYVINYSKLRIWEFVEYSKMIYLDGDIQVYDNIDHLFDLPDGHFYAVMDCFCEKTWSHTPQYKIGYCQQCPDKVNWPAEMGQPPSLYFNAGMFVFEPSISTYHDLLKTLKVTPPTPFAEQDFLNMYFKDIYKPIPLVYNLVLAMLWRHPENVELDKVKVVHYCAAGSKPWRYTGKEENMQREDIKMLVEKWWGIYNDESLDYMKFVADGFDAEPVNLQSFIAALSEAGAVQYVTAPSAA

SEQ ID NO: 19 - protein, 325 aa, Populus Trichocarpa
  GolS8 protein sequence
MSPNSIIEPTTDLQKRAYVTFLAGNGDYVKGVVGLAKGLRKVKSAYPLVVAILPDVPEEHREILESQGCIIREIEPLYPPENQTQFSMAYYVINYSKLRIWEFVDYGKMIYLDGDIQVFDNIDHLFEKPTGYFYAVMDCFCEKTWSTTPQYQIKYCQQCPEKVQWPLEMGSPPPLYFNAGMCLFEPKLETYFDLLETLKVTTPTSFAEQDFLNMFFRDVYQPIPPVYNLVSAMLWRHPDKFDLDKVKVVHYCAAGAKPWRYTGKEENMDREDIKVLVKKWWEIYEDESLDFKNATVPVAQEKLGPLIAALTEDGVGNHMNLPSAA

SEQ ID NO: 20 - protein, 336 aa, Populus Trichocarpa
  GolS2 protein sequence
MAPGVPMDVISCTGKVSTASTGYSKRAFVTFLAGNGDYVKGVVGLAKGLRKVKSAYPLVVAMLPDVPEEHRDILRSQGCIVREIEPIYPPENQIQFAMAYYVINYSKLRIWNFEEYSKMMYLDADIQVFENIDHLFDTQDGYFYAVMDCFCEKTWSHSPQHSIGYCQQCPEKVTWPAEMGSPPPLYFNAGMFVFEPSRLTYESLLETLQITPPTPFAEQDFLNMFFQKTYKPIPLMYNLVLAMLWRHPENVEVEKVKVVHYCAAGSKPWRYTGKEANMDREDIKMLVARWWDIYNNESLDFKAENSVPEEETLSRSSILSSKPEPAISYISAPSAA

SEQ ID NO: 21 - protein, 337 aa, Populus Trichocarpa
  GolS6 protein sequence
MAPELVRSALKPAGFTKLANLPSRAYVTFLAGDGDYVKGVVGLAKGLRKVKTAYPLIVAVLPDVPEEHRQILESQGCIVREIEPVYPPENQTQFAMAYYVINYSKLRIWEFVEYSKMIYLDGDIQVYDNIDHLFDLPDGRFYAVMDCFCEKTWSHTPQYKIGYCQQCPDKVNWPAEMGQPPSPYFNAGMCVFEPSIATYHDLLKTLKVTPPTPFAEQDFLNMYFKDIYTPIPLVYNLVLAMLWRHPENVELDRVKVVHYCAAGSKPWRYTGKEENMQREDIKMLVKKWWDIYSDESLDSKKLVADCTTDAEPVNLQPFIAALSEAGAVQYVTAPSAA

SEQ ID NO: 22 - protein, 338 aa, Populus Trichocarpa
  GolS1 protein sequence
MAPGVPIDGNILGTGKVSTVNTGYSKRAYVTFLAGNGDYVKGVVGLAKGLRKVKSAYPLVVAILPDVPEEHRDILRSQGCIVREIEPIYPPENQIQFAMAYYVINYSKLRIWNFEEYSKMIYLDADIQVFENIDHLFDTQDGYFYAVMDCFCEKTWSHSPQYSVGYCQQCPEKITWPAEMGSPPPLYFNAGMFVFEPSRLTYESLLERLQITPPTPFAEQDFLNMFFQKTYKPIPLLYNLVLAMLWRHPENVEVEKVKVVHYCAAGSKPWRYTGEEANMDREDIKMLVAKWWDIYNDESLDFNGENSVPEEETIFSRSSILSSMPEPAISYVPAPTAA

SEQ ID NO: 23 - protein, 325 aa, Populus Trichocarpa
  GolS9 protein sequence
MSPNAIIEPTFNSHKRAYVTFLAGNGDYVKGVVGLAKGLRKAKSSYPLMVAILPDVPEEHRKILESQGCIVREIEPVYPPDNQTQFAMAYYVINYSKLRIWEFVDYEKMIYLDGDIQVFDNIDHLFDEPNGYFYAVMDCFCEKTWSSTPQYQIGYCQQCPEKVRWPKEMGSPPPLYFNAGMFVFEPKLLTYFDLLETLKVTPPTSFAEQDFLNMFFRDVYKPIPAVYNLVSAMLWRHPENFELDKVKVVHYCAAGAKPWRYTGKEENMDREDIQVLVKKWWEIYEDESLDYKNITVPVDQEKLGPLIAALTDDGVINHRNLPSAA

SEQ ID NO: 24 - protein, 337 aa, P. albaxgrandidentata hybrid poplar
  GolSI protein sequence
MAPGVPMDVISCTGKVSTASHGYSKRAFVTFLAGNGDYVKGVVGLAKGLRRVKSAYPLVVAMLPDVPEEHRDILRSQGCIVREIEPIYPPENQIQFAMAYYVINYSKLRIWNFEDYSKMMYLDADIQVFENIDHLFDTQDGYFYAVMDCFCEKTWSHSPQYSIGYCQQCPEKVTWPAEMGSPPPLYFNAGMFVFEPSRLTYESLLETLQITPPTPFAEQDLLNMFFQKTYKPIPLMYNLVLAMLWRHPENVEVEKVKVVHYCAAGSKPWRYTGKEANMDREDIKMLVARWWDIYNNESLDFKAENSVPDQEETLSRSSILSSKPGPAISYISAPSAA

SEQ ID NO: 25 - protein, 337 aa, P. albaxgrandidentata hybrid poplar
  GolsSII protein sequence
MAPHITTTLANTTNSLVKQASLSSCAYVTFLAGDGDYWKGVVGLAKGLRKAKSKYPLVVAILPDVPEEHRMILVSQGCIVREIEPVHPPENQTRFAMPYYVINYSKLRIWEFVEYSKMIYLDGDIQVFDNIDHLFDMPDGHFYAVMDCFCEKTWSNSPQYKIGYCQQCPDKVQWPAEMGPKPPLYFNAGMFVYEPNLSTYHDLLETVKVTSPTLFAEQDFLNMFFRDVYKPIPSDYNLVLAMLWRHPENINLDKVKVVHYCAAGSKPWRFTGKEENMDREEIKMVVNKWWDIYQDESLDYKNTVAAATASAGAELHPFLAALSEAGVVHYVTAPSAA

SEQ ID NO: 26 - DNA, 1014 bp, Populus Trichocarpa
  GolS3 nucleotide sequence
atggctcctcatattacaactgcccttgctaacagcaccaactctctagtcaagcaagctagcttgtcgagctgtgcatatgttacctttttggctggtgatggagactactggaagggtgttgtggggttagccaaggggctgaggaaggccaagagcaagtatcctttagtggtggctatcttgcctgatgttcctgaggagcatcggatgatacttgtctctcaagggtgtatagtgagggagattgagcctgttcatccaccggagaaccagactcagtttgctatgccttattatgtcatcaactactccaagcttcgtatctgggagtttgttgagtatagcaagatgatatatttggacggggacatccaggtatttgacaacatagaccacctgttcgacatgcctgatggttacttctatgctgtgatggactgtttctgtgagaaaacatggagcaacagccctcagtacaagattggttactgccaacagtgtcctgataaggtccagtggcctgctgagatgggtcctaagccccctctgtacttcaatgctggcatgtttgtttatgagcccaacttgtccacttatcatgatctgttggagaccgtcaaagtcacctcccccaccctcttcgctgagcaggacttcttgaacatgtttttcagggatgtttacaagccaattccttcggattacaaccttgtactggccatgttatggcgccatcctgagaacattaaccttgacaaagtgaaagttgtccactattgcgctgctgggtctaagccatggaggttcactggaaaagaagagaacatggacagagaagacatcaagatggtggtcaacaaatggtgggacatttaccgggacgaatcattggactacaagaatactgttgctgctgctgctgcttctgctggagctgaactgcatccattcttggcggctctctctgaggctggtgttgtccactatgttaccgccccatccgccgcttag

SEQ ID NO: 27 - DNA, 1014 bp, Populus Trichocarpa; identical to SEQ ID NO: 26
  GolS3 nucleotide sequence

SEQ ID NO: 28 - DNA, 1014 bp, Populus Trichocarpa
  GolS4 nucleotide sequence
atggctcctgagcttgtacggtctgctttgaaaccagccgggttcaccaagctcgctagcttacctagcagggcttatgtaacgttcttggctggtgatggagattacgtgaaaggcgttgtcgggctagccaaaggcttaagaaaggtcaaaacggcatatccattaatcgtggcagtcttacctgacgtgccagaggaacacaggcagatcttggaatctcaagggtgtatagtccgggaaattgaacccgtttacccgccagagaaccagacccagtttgccatggcttactatgtcatcaactactccaagctccgtatatgggagtttgtggaatacagcaagatgatatacttggatggagatatccaggtttatgacaacatagaccacttatttgatctgcctgatgggcgtttttatgcggtgatggattgtttctgtgagaaaacatggagtcacactccgcagtacaagattggctattgccagcaatgccctgacaaggttaactggccagctgagatgggtcaaccaccatctccttacttcaatgctggcatgtgtgtgtttgagcctagtattgccacttatcatgatctcttgaagactctcaaagtcactcctcctaccccatttgcggagcaggactttttgaacatgtacttcaaggatatttacacgccaattcctctagtttacaatcttgttcttgccatgctatggcgtcacccagaaaatgtggagcttgacagggtcaaagttgttcactactgtgctgcgggctcgaagccttggagatacactggcaaagaagagaacatgcagagagaggatataaagatgctggtgaagaaatggtgggacatttacagtgatgaatcactggactccaagaaacttgtggcggattgtactactgatgctgagccagtgaacttacatccattcattgcagctctctctgaagccggcgctgttcaatatgtcactgccccatctgctgcctag

SEQ ID NO: 29 - DNA, 978 bp, Populus Trichocarpa
  GolS nucleotide sequence
atgtctcccaattccataatcgagcctacgacggacttgcagaagagagcatatgttacgttcttggccggaaacggtgattatgtgaaaggtgtggtgggtttagccaagggtttaagaaaagtgaaatctgcataccctcttgtggtggctattttgcctgatgtgcccgaggagcaccgcgagattctagaatctcagggctgtatcatccgtgagattgagcccctgtatccaccagagaaccagactcagttttccatggcttattatgtgattaactactcaaaacttcgtatttgggagtttgtggactatgggaagatgatatacttggatggtgatattcaagtctttgacaatattgatcatcttttcgagaagccaactggatatttttatgctgtgatggactgtttttgtgagaagacatggagtaccactcctcagtatcagatcaaatactgccagcaatgccctgaaaaagttcagtggcctttggagatgggctctccccctcctctatactttaatgctggcatgtgtctcttcgaacctaagctcgagacttactttgatctcttggagaccctcaaagtcaccacccctacctcatttgctgagcaggattttctgaacatgttcttcagggatgtataccagcctatccctcctgtctacaaccttgtttcggctatgctgtggcgccatcccgataaattcgaccttgacaaagtgaaggttgtccactattgtgctgcgggtgcaaagccatggaggtatactggaaaagaagaaaacatggatcgagaggacatcaaggttttggtgaaaaaatggtgggaaatatatgaggatgagtccctggatttcaagaatgccactgtgcctgttgctcaagaaaaacttggacccctaatagcggcattgactgaggatggggtgggtaaccacatgaatctaccatctgcagcttaa

SEQ ID NO: 30 - DNA, 1011 bp, Populus Trichocarpa
  GolS2 nucleotide sequence
atggctccaggagtgcccatggatgtgatttcctgcaccggcaaggtttccacggctagcactggctactctaaaagggcctttgtaacatttttagccggaaatggggattatattaaaggggtagttgggttggcaaagggtttgcgcaaggtgaaaagtgcataccctctggtggtagcaatgttgccagatgtgcctgaggaacaccgcgacattttgaggtctcaaggttgcattgttcgtgagattgagcctatttatccacctgagaaccagattcagtttgctatggcttattacgtgatcaactactccaagctccgcatttgggatttcgaggagtacaccaagatgatgtatttggatgctgatatccaagtgttcgagaatatagaccatctatttgacacccaagatggctacttctatgccgtgatggactgcttctgtgagaaaacctggagccactcaccacaacactctatcggttactgccagcagtgcccggaaaaggtgacatggcctgctgagatgggctctcctcctccattgtacttcaatgctgggatgtttgtctttgagcctagtcggttgacttatgagagccttcttgaaactctccagattaccccaccaaccccatttgccgagcaagatttcttaaatatgtttttccaaaaaacatacaagccaatccctctaatgtacaacctggttttagccatgttatggcgacatcctgagaatgtggaggtcgaaaaggttaaagtggttcactactgtgctgctggttcaaaaccttggagatatactggcaaggaagctaacatggacagagaggacatcaagatgctagtggcaagatggtgggacatatataataacgagtccctcgatttcaagggtgagaactcagttccagaagaagaaacattgtcgaggtcatccatcttgtcttccaagcctgagcctgccatttcctacatctctgcgccatctgctgcttaa

SEQ ID NO: 31 - DNA, 1346 bp, Populus Trichocarpa
  GolS1 nucleotide sequence
aattcggcacgaggacaatcctcaaaaacattcttcttagactctctatacattccaaataacctaatttgtactgtatatagatatatatatagatatatagtctacgtcaagcttgaataaatcttcattaacatggccccaggagtgcctatagatgggaatatttttggtaccgggaaggtttccacagttaacactggctattctaagagggcctacgtgacatttttagctggcaacggggattatgttaaaggggtagttgggttggctaagggtttgcgcaaggtgaagagtgcataccctcttgtcgtagcaatcttgccggacgtgcctgaggaacaccgtgacattttaaggtctcaaggttgcattgttcgtgagatcgagcctatttatccacctgagaaccagattcagtttgccatggcctactacgtgatcaactactctaagctccgaatttggaattttgaggagtacagcaagatgatatatttggatgctgatatccaagtgttcgagaatatagaccatctatttgacacccaagatggctacttctacgccgtgatggactgcttctgtgagaaaacatggagccactcccctcaatactccgtcggctactgccagcagtgcccggaaaagataacatggcctgctgagatgggctctcctcctcccctgtacttcaatgctgggatgtttgtatttgagcctagtcgtttgacttatgagagccttcttgagaggctgcagatcaccccaccaaccccctttgccgagcaagatttcttgaatatgtttttccaaaaaacatacaagccaatccctctgctttacaacctcgttttagccatgctatggcgacatcctgagaacgtggaggtcgagaaggttaaagtggttcactactgtgctgctggttcaaaaccttggagatatactggcgaggaagctaacatggatagagaggacatcaagatgttagtggcaaaatggtgggatatatataatgatgagtccctcgatttcaatggcgaaaactcagttccagaagaagaaacaatattttcgaggtcatccatcttgttttccatgcctgagcctgccatatcctacgtccctgcaccaactgctgcttagaactttttagttaagagagtttaaaatcaatcttgtctttcattatatacctttttcaggttatttcgcctgagtattttcgtgttttgtgcaagatatataccgtggtaatatataaagtattacagtatttgaacttttatattaaaaaaaaaaaaaaaaaaaaaaaaataaaaaaaaaaaaaaaaaaaaaa

SEQ ID NO: 32 - DNA, 1337 bp, Populus Trichocarpa
  GolS2 nucleotide sequence
aattcggcacgaggtgctggtttactttcctaagaaacctcaagtacaccaatcttctttgcctctctaatacattccaaataaccttgtactatattcatagtatttattaagcttcaaaaatctcaagtactatggctccaggagtgcccatggatgtgatttcctgcaccggcaaggtttccacggctagcactggctactctaaaagggcctttgtaacatttttagccggaaatggggattatattaaaggggtagttgggttggcaaagggtttgcgcaaggtgaaaagtgcataccctctggtggtagcaatgttgccagatgtgcctgaggaacaccgcgacattttgaggtctcaaggttgcattgttcgtgagattgagcctatttatccacctgagaaccagattcagtttgctatggcttattacgtgatcaactactccaagctccgcatttgggatttcgaggagtacaccaagatgatgtatttggatgctgatatccaagtgttcgagaatatagaccatctatttgacacccaagatggctacttctatgccgtgatggactgcttctgtgagaaaacctggagccactcaccacaacactctatcggttactgccagcagtgcccggaaaaggtgacatggcctgctgagatgggctctcctcctccattgtacttcaatgctgggatgtttgtctttgagcctagtcggttgacttatgagagccttcttgaaactctccagattaccccaccaaccccatttgccgagcaagatttcttaaatatgtttttccaaaaaacatacaagccaatccctctaatgtacaacctggttttagccatgttatggcgacatcctgagaatgtggaggtcgaaaaggttaaagtggttcactactgtgctgctggttcaaaaccttggagatatactggcaaggaagctaacatggacagagaggacatcaagatgctagtggcaagatggtgggacatatataataacgagtccctcgatttcaagggtgagaactcagttccagaagaagaaacattgtcgaggtcatccatcttgtcttccaagcctgagcctgccatttcctacatctctgcgccatctgctgcttaaacttcttgattaagatagtttaaaaactggtgtaatcccatccttcctttaattttgtctttcatcataaaccctttttcaggctgcttgttctgtgactttacacgcaagacactgtggtgttatatatggtgttcttgtatttgaaatagtattcatacgaatctctattggcaaaaaaaaaaaaaaaaa

SEQ ID NO: 33 - DNA, 1380 bp, Populus Trichocarpa
  GolS4 nucleotide sequence
aattcggcacgaggcaacagccagctttcctctccatttcttctctcaagttctcttttaaagcctcctctctcactctctttcatctccctcttcgacgaccgcaacatggctcctgagcttgtacggtctgctttgaaaccagccgggttcaccaagctcgctagcttacctagcagggcttatgtaacgttcttggctggtgatggagattacgtgaaaggcgttgtcgggctagccaaaggcttaagaaaggtcaaaacggcatatccattaatcgtggcagtcttacctgacgtgccagaggaacacaggcagatcttggaatctcaagggtgtatagtccgggaaattgaacccgtttacccgccagagaaccagacccagtttgccatggcttactatgtcatcaactactccaagctccgtatatgggagtttgtggaatacagcaagatgatatacttggatggagatatccaggtttatgacaacatagaccacttatttgatctgcctgatgggcgtttttatgcggtgatggattgtttctgtgagaaaacatggagtcacactccgcagtacaagattggctattgccagcaatgccctgacaaggttaactggccagctgagatgggtcaaccaccatctccttacttcaatgctggcatgtgtgtgtttgagcctagtattgccacttatcatgatctcttgaagactctcaaagtcactcctcctaccccatttgcggagcaggactttttgaacatgtacttcaaggatatttacacgccaattcctctagtttacaatcttgttcttgccatgctatggcgtcacccagaaaatgtggagcttgacagggtcaaagttgttcactactgtgctgcgggctcgaagccttggagatacactggcaaagaagagaacatgcagagagaggatataaagatgctggtgaagaaatggtgggacatttacagtgatgaatcactggactccaagaaacttgtggcggattgtactactgatgctgagccagtgaacttacatccattcattgcagctctctctgaagccggcgctgttcaatatgtcactgccccatctgctgcctagagatgggaagaaagaaggatcaaagcactattatagatttagaagggatcaaagttggaatccttctcttgatagtaatttcttgggtatagaaaagagatacactatcttctttcttggtagtatctctattttccactctatccctctgctgattatgggtggagcaattgttaaattaatgcaaataaacatatgttgtgttacctagtttaattcactccctcttttcttgtttttttaaaaaaaaaaaaaaaa

SEQ ID NO: 34 - DNA, 1454 bp, Populus Trichocarpa
  GolS3 nucleotide sequence
aattcggcacgaggctttgaagagcaaagcagcatacttttctgcaaccttaaattctctctcttttttctcttagcaaacaaatttgtttcaaagcccttttgatttctcttaaccgaagactcattttttgatagctttgataccatggctcctcatattacaactgcccttgctaacagcaccaactctctagtcaagcaagctagcttgtcgagctgtgcatatgttacctttttggctggtgatggagactactggaagggtgttgtggggttagccaaggggctgaggaaggccaagagcaagtatcctttagtggtggctatcttgcctgatgttcctgaggagcatcggatgatacttgtctctcaagggtgtatagtgagggagattgagcctgttcatccaccggagaaccagactcagtttgctatgccttattatgtcatcaactactccaagcttcgtatctgggagtttgttgagtatagcaagatgatatatttggacggggacatccaggtatttgacaacatagaccacctgttcgacatgcctgatggttacttctatgctgtgatggactgtttctgtgagaaaacatggagcaacagccctcagtacaagattggttactgccaacagtgtcctgataaggtccagtggcctgctgagatgggtcctaagccccctctgtacttcaatgctggcatgtttgtttatgagcccaacttgtccacttatcatgatctgttggagaccgtcaaagtcacctcccccaccctcttcgctgagcaggacttcttgaacatgtttttcagggatgtttacaagccaattccttcggattacaaccttgtactggccatgttatggcgccatcctgagaacattaaccttgacaaagtgaaagttgtccactattgcgctgctgggtctaagccatggaggttcactggaaaagaagagaacatggacagagaagacatcaagatggtggtcaacaaatggtgggacatttaccgggacgaatcattggactacaagaatactgttgctgctgctgctgcttctgctggagctgaactgcatccattcttggcggctctctctgaggctggtgttgtccactatgttaccgccccatccgccgcttagtgcttgtaaattgtggagctactagtttcaatcagtccaattgtagaggaaaaacaacttttctttcagctctttttggtggttaacatcccctatattgttaacagggatgggatattgctcttttgaattttctaggaagtttatgaggaccttgtatccatcctatagtaaataatatgaacaagtattttctttgtatacacatttcttttatcttttcttatgttgtattgaagaagcaataatgggatttgctctgactaacttctatttttcaaaaaaaaaaaaaa

SEQ ID NO: 35 - DNA, 1278 bp, Populus Trichocarpa
  GolS2 nucleotide sequence
agaaacctcaagtacaccaatcttctttgcctctctaatacattccaaataaccttgtactatattcatagtatttattaagcttaaaaaatctcaagtactatggctccaggagtgcccatggatgtgatttcctgcaccggcaaggtttccacggctagcactggctactctaaaagggcctttgtaacatttttagccggaaatggggattatgttaaaggggtagttgggttggcgaagggtttgcgcaaggtgaagagtgcataccctctggtggtagcaatgttgccagatgtgcctgaggaacaccgtgacattttgaggtctcaaggttgcattgttcgtgagattgagcctatttatccacctgagaaccagattcagtttgccatggcctactacgtgatcaactactccaagctccgcatttggaatttcgaggagtacagcaagatgatgtatttggatgctgatatccaagtgttcgagaatatagaccatctatttgacacccaagatggctacttctatgccgtgatggactgcttctgtgagaaaacctggagccactcaccacaacactctatcggttactgccagcagtgcccggaaaaggtgacatggcctgctgagatgggctctcctcctcccttgtacttcaatgctgggatgtttgtctttgagcctagtcgtttgacttatgagagccttcttgaaactctccagattaccccaccaaccccatttgccgagcaagatttcttaaatatgtttttccaaaaaacatacaagccaatccctctaatgtacaacctggttttagccatgttatggcgacatcctgagaatgtggaggtcgaaaaggttaaagtggttcactactgtgctgctggttcaaaaccttggagatatactggcaaggaagctaacatggacagagaggacatcaagatgctagtggcaagatggtgggacatatataataacgagtccctcgatttcaaggctgagaactcagttccagaagaagaaacattgtcgaggtcatccatcttgtcttccaagcctgagcctgccatttcctacatctctgcgccatctgctgcttaaacttctcgattaagatagtttaaaaactggtgtaatcccatccttcctttaattttgtctttcatcataaaccctttttcaggctgcttgtgctaattattttcttgttctgtgactttacatgcaagacactgtggtgttatttaaaaaaaaaaaaaaaaaaaa

SEQ ID NO: 36 - DNA, 1277 bp, Populus trichocarpa X Populus deltoides
  GolS1 nucleotide sequence
acaatcctcaaaaccattcttcttagactctctatacattccaaataacctaatttgtactgtatagatatatagtctacgtcaagcttaaataaatcctcattaacatggccccaggagtgcctatagatgggaatattttgggtaccgggaaggtttccacagttaacactggctattctaagagggcctacgtgacatttttagccggcaacggggattatgttaaaggggtagttgggttggctaagggtttgcgcaaggtgaagagtgcataccctcttgtcgtagcaatcttgccggatgtgcccgaggaacaccgtgacattttgaggtctcaaggttgcattgttcgtgagatcgagcctatttatccacctgagaaccagattcagtttgccatggcctactacgtgatcaactactccaagctccgaatttggaattttgaggagtacagcaagatgatatatttggatgctgatatccaagtgttcgagaatatagaccatctatttgacacccaagatggctacttctacgccgtgatggactgcttctgtgagaaaacatggagccactcccctcaatactccgtcggctactgccagcagtgcccggaaaagataacatggcctgctgagatgggctctcctcctcccctgtacttcaatgctgggatgtttgtatttgagcctagtcgtttgacttatgagagccttcttgagaggctgcagatcaccccaccaaccccctttgccgagcaagatttcttgaatatgtttttccaaaaaacatacaagccaatccctctgctttacaacctggttttagccatgttatggcgacatcctgagaacgtggaggtcgagaaggttaaagtggttcactactgtgctgctggttcaaaaccttggagatatactggcgaggaagctaacatggatagagaggacatcaagatgttagtggcaaaatggtgggatatatataatgatgagtccctcgatttcaatggtgaaaactcagctccagaagaagaaacaatattctcgaggtcatccatcttgtcttccatgcctgagcctgccatatcctacgtccctgcaccaactgctgcttaaaacttcttagttaagagagtttaaaatcaatcttgtctttcattatatacctttttcaggctacttcgcctgagtattttcgtgttctgtgcaagatatataccatggtaatatataaagtattacagtatttgaacaaaaaaaaaaaaaaaa

SEQ ID NO: 37 - DNA, 1269 bp, Populus Trichocarpa
  GolS1 nucleotide sequence
acaatcctcaaaaccattcttcttagactctctatacattccaaataaccaaatttgtactgtatagatatatagtctacgtcaagcttaaataaatcctcattaacatggccccaggagtgcctatagatgggaatattttgggtaccgggaaggtttccacagttaacactggctattctaagagggcctacgtgacatttttagccggcaacggggattatgttaaaggggtagttgggttggctaagggtttgcgcaaggtgaagagtgcataccctcttgtcgtagcaatcttgccggatgtgcccgaggaacaccgtgacattttgaggtctcaaggttgcattgttcgtgagatcgagcctatttatccacctgagaaccagattcagtttgccatggcctactacgtgatcaactactccaagctccgaatttggaattttgaggagtacagcaagatgatatatttggatgctgatatccaagtgttcgagaatatagaccatctatttgacacccaagatggctacttctacgccgtgatggactgcttctgtgagaaaacatggagccactcccctcaatactccgtcggctactgccagcagtgcccggaaaagataacatggcctgctgagatgggctctcctcctcccctgtacttcaatgctgggatgtttgtatttgagcctagtcgtttgacttatgagagccttcttgagaggctgcagatcaccccaccaaccccctttgccgagcaagatttcttgaatatgtttttccaaaaaacatacaagccaatccctctgctttacaacctggttttagccatgttatggcgacatcctgagaacgtggaggtcgagaaggttaaagtggttcactactgtgctgctggttcaaaaccttggagatatactggcgaggaagctaacatggatagagaggacatcaagatgttagtggcaaaatggtgggatatatataatgatgagtccctcgatttcaatggcgaaaactcagttccagaagaagaaacaatattctcgaggtcatccatcttgtcttccatgcctgagcctgccatatcctacgtccctgcaccaactgctgcttaaaacttcttagttaagagagtttaaaatcaatcttgtctttcattatatacctttttcaggctacttcgcctgagtattttcgtgttctgtgcaagatatataccatggtaatatataaagtattacagtaaaaaaaaaaaaaaaa

SEQ ID NO: 38 - protein, 337 aa, P. albaxgrandidentata hybrid poplar
  GolSI protein sequence
MAPGVPMDVISCTGKVSTASHGYSKRAFVTFLAGNGDYVKGVVGLAKGLRRVKSAYPLVVAMLPDVPEEHRDILRSQGCIVREIEPIYPPENQIQFAMAYYVINYSKLRIWNFEDYSKMMYLDADIQVFENIDHLFDTQDGYFYAVMDCFCEKTWSHSPQYSIGYCQQCPEKVTWPAEMGSPPPLYFNAGMFVEPSRLTYESLLETLQITPPTPFAEQDLLNMFFQKTYKPIPLMYNLVLAMLWRHPENVEVEKVKVVHYCAAGSKPWRYTGKEANMDREDIKMLLVARWWDIYNNESLDFKAENSVPDQEETLSRSSILSSKPGPAISYISAPSAA

SEQ ID NO: 39 - protein, 333 aa, Arabidopsis thaliana
  GolS5 protein sequence
MTMTVEKRIEADVTVSHEGVERAYVTFLAGNKDYWMLVVGLAKGLRKVKSAYPLVVATLPDVPEEHRQILVDQGCIIRDIEPVYPPENTTGYSMAYYVINYSKLRIWEFVEYEKMIYLDGDIQVFKNIDHLFDTPRGYLYAVKDCFCEVSWSKTPQYKIGYCQQSPEKVTWPVESLGAPPPVYFNAGMLVFGPNLVTYEDLLRVVQITTPTYFAEQDFLNIYFRDIYKPIPSTYNLVMAMLWRHPEHIDLDQISVVHYCANGSKPWKFDEAEEHMDREDIKMLVKKWWEIYEDSSLDYKNFVETESKLNPVTATLASKKLVGDVLTSLAPSAA

SEQ ID NO: 40 - protein, 328 aa, Oryza sativa
  GolS1 protein sequence
MMGPNVSSEKKALAAAKRRAYVTFLAGDGDYWKGVVGLAKGLRRVRSAYPLVVAVLPDVPGEHRRKLVEQGCVVREIQPVYPPESQTQFAMAYYVINYSKLRIWEFVEYERMVYLDADIQVFDNIDHLFDLDKGAFYAVKDCFCEKTWSHTPQYDIGYCQQRPDEVAWPERELGPPPPLYFNAGMFVHEPGLGTAKDLLDALVVTPPTPFAEQDFLNMFFREQYKPIPNVYNLVLAMLWRHPENVDLDQVKVVHYCAAGSKPWRFTGKEENMNREDIKMLVKRWWDIYNDESLDYKEEEDNADEASQPMRTALAEAGAVKYFPAPSAA

SEQ ID NO: 41 - protein, 331 aa, Cucumis melo
  GolS1 protein sequence
MSPAAAPESAIESTDAPKRAYVTFLAGNGDYWKGVVGLAKGLRKVKAAYPLIVAVLPDVPEDHRQILEYQGCVVREIEPVYPPENQTQFAMAYYVINYSKLRIWEFVEYEKLIYLDGDIQVFENIDHLFAMPSGYFYAVMDCFCEKTWSNSPQYKIGYCQQCPDKVKWPVEEMGNPPPLYFNAGFFVYEPDLFTYKDLLETCKATTPTLFAEQDFLNMYFNDIYKPIPPIYNLVMAMLWRHPENIDVDKVKVVHYCAAGSKPWRYTGKEENMDREDIKMLVKKWWEVYEDESLDYQNVLKSETKQETNLTPLISVLSEAEVVNHITAPSAA

SEQ ID NO: 42 - protein, 292 aa, Ajuga reptans
  GolS2 protein sequence
VGLAKGLRKVGTIYPLVVAVLPDVPPEHRRILVEQGCVVREIEPVYPPENHTEFAMAYYVINYSKLRIWEFVEYSKMIYLDGDIQVFENIDHLFDLENGYFYAVMDCFCEKTWSHTPQYQIGYCQQSPKRVHWPKQLGPKPPLYFNAGMFVYEPSLPTYHDLLHTLKITPPTPFAEQDFLNMFLRDVYRPIPNVYNLVLAMLWRHPENVNLEAVKVVHYCAAGSKPWRYTGEEENMDRNDIKMLVNKWRDIYDDEMLDYNAVADPAADGLQLTAVLTEAAGVVRFIPAPSAA

SEQ ID NO: 43 - protein, 333 aa, Ajuga reptans
  GolS1 protein sequence
MGPVVPVEAFRSAGKISALGAKKGYVTFLAGNGDYVKGVVGLAKGLRKVKSAYPLVVAILPDVPEEHRELLRSQGCIVKEIEPIYPPANQIQFAMAYYVINYSKLRIWNFEEYSKMVYLDADIQVYENIDHLLDTPDGYFYAVMDCFCEKTWSHSRQFSIGYCQQCPNKVTWPAQMGSPPPLYFNAGMFVFEPSKTTYQTLLHTLRITPPTPFAEQDFLNMFFEPIYKPIPLVYNLVLAMLWRHPENVELEKVQVVHYCAAGSKPWRYTGQEANMDREDIKMLVKKWWDVYNDESLDFKAEDSIAGEETFSMPSFIASLPEPAVSYIPAPSAA

SEQ ID NO: 44 - protein, 344 aa, Arabidopsis thaliana; identical to SEQ ID NO: 6
  GolS1 protein sequence

SEQ ID NO: 45 - protein, 337 aa, P. albaxgrandidentata hybrid poplar; identical to SEQ ID NO: 25
  GolSII protein sequence

(19) United States
(12) Patent Application Publication
(10) Pub. No.: US 2014/0289903 A1
(43) Pub. Date: Sep. 25, 2014

(54) ENHANCING CELL WALL PROPERTIES IN PLANTS OR TREES

(71) Applicants: Shawn D. Mansfield, Vancouver (CA); Faride G. Unda, Vancouver (CA)

(72) Inventors: Shawn D. Mansfield, Vancouver (CA); Faride G. Unda, Vancouver (CA)

(73) Assignee: THE UNIVERSITY OF BRITISH COLUMBIA, Vancouver, BC (CA)

(21) Appl. No.: 14/127,000

(22) PCT Filed: Jun. 29, 2012
(86) PCT No.: PCT/CA2012/050450

§ 371 Date: Jan. 27, 2014

Related U.S. Application Data
(60) Provisional application No. 61/502,509, filed on Jun. 29, 2011.

(51) Int. Cl. C12N 15/82; A23K 1/14; A23L 1/212; C10L 5/44; C12N 9/00

(52) U.S. Cl. CPC C12N 15/8261; C10L 5/44; C12N 9/93; A23L 1/2123; A23K 1/14
USPC 800/290; 800/298; 435/419; 44/605; 536/23.2; 426/615; 426/635

(57) ABSTRACT

The present invention relates to a method for enhancing cell wall properties in plants. The method comprises introducing into the plant, at least one nucleotide construct comprising a nucleic acid molecule operatively linked to a regulatory region active in the plant, wherein said nucleic acid molecule encodes a polypeptide with galactinol synthase (GolS)-like activity and growing the plant under conditions that permit the expression of the nucleic acid, thereby enhancing the cell wall property of the plant.

### FIELD OF INVENTION

This invention relates to enhancing cell wall properties in plants, perennial plants, or trees. More specifically, the invention relates to the expression of an enzyme with galactinol synthase (GolS), or GolS-like, activity in plants, perennial plants or trees to enhance cell wall properties in the plant or tree.

### BACKGROUND OF THE INVENTION

There is an increased interest in the use of renewable biomass for biofuel production as an environmental friendly and socio-economically responsible fuel alternative. Along with what is derived from sugarcane, a significant amount of ethanol is generated from grain-derived starch, such as maize, throughout North America. Although established, such production is not likely the best long-term strategy, since the current agricultural-derived capacity is not sufficient to sustainably produce the substantial projected requirements. Equally important, there is an inherent competition for land use for food production. In contrast, a promising source of ethanol is the abundant lignocellulosic feedstocks, including wood and fiber-derived biomass, produced from forested lands and marginal agricultural lands. Lignocellulosic biomass, which is available in a number of forms, represents an abundant, inexpensive, and generally locally available feedstock.

Lignocellulosic feedstocks are chemically and structurally more complex than the currently employed substrates such as soluble sugars derived from sugar cane or starch in corn-derived ethanol. Lignocellulosic feedstocks consist of plant cell walls composed of chemically linked polymeric macromolecules composed of cellulose, lignin, and hemicelluloses. The structure and chemistry of woody feedstocks inherently make these substrates recalcitrant to breakdown into fermentable sugars, owing to the compact structure of crystalline cellulose microfibrils, the lack of substrate porosity and the presence of higher lignin concentrations (Chang & Holtzapple, 2000 Applied Biochemistry and Biotechnology 84-86: 5-37).

Approximately 70% of plant biomass is estimated to be present in plant cell walls and currently only about 2% of plant cell wall-based biomass are used. There is therefore an opportunity to use this resource as a raw material for the production of biofuels and as commodity chemicals. The plant cell wall provides mechanical support to the plant and contributes to plant growth and development. Carbohydrates, proteins and phenolic compounds (e.g., lignin) are the major components in the plant cell wall with cellulose, hemicellulose and pectin comprising the major polysaccharides.

Carbohydrates are key-players in a multitude of fundamental physiological events in plants, such as development, signaling, carbon transport and storage, cell wall synthesis, and stress protection. Sources of translocatable soluble carbohydrates include sucrose, as well as the water-soluble raffinose family oligosaccharides (RFOs) which are α-1,6 galactosyl extensions of sucrose (Suc) with the most common species being raffinose (Suc-Gal1), stachyose (Suc-Gal2), and verbascose (Suc-Gal3). RFOs are the most abundant oligosaccharides in the plant kingdom and many RFO-producing plants are of economic importance. As non-reducing carbohydrates they are good storage compounds that can accumulate in large quantities without affecting primary metabolic processes. The potential role of RFOs in stress tolerance has been intensively studied in seeds, mainly with respect to desiccation tolerance and longevity in the dehydrated state. Additionally, RFO accumulation has commonly been associated with abiotic stress conditions such as cold, heat or drought in several plant species.

US 2004/0019932 and U.S. Pat. No. 7,294,756 describe altering raffinose saccharide synthesis in legume seeds using Glycine max galactinol synthase (GolS), in order to enhance nutritional qualities of edible seed of leguminous plants. U.S. Pat. No. 5,648,210 discloses the GolS sequence from zucchini and soybean and the use of these sequences to alter the soluble carbohydrate composition in Brasica napus seed. Three times greater activity of galactinol synthase was observed in transgenic seeds when compared to wild-type seed. Despite the increased amount of galactinol synthase activity, the total alpha-galactoside content of the transformed lines was significant less than that of the wild-type. More specifically, the transformed plants showed a reduction in the raffinose saccharide content and an increase in sucrose content.

### SUMMARY OF THE INVENTION

This invention relates to enhancing cell wall properties in plants, perennial plants or trees. The invention further relates to altering level of in a plant, perennial plant or tree. More specifically, the invention relates to the expression of an enzyme with galactinol synthase (GolS), or GolS-like, activity in plants, perennial plants or trees to enhance cell wall properties in the plant, perennial plant or tree and/or to alter level of carbohydrates in the plant, perennial plant or tree.

The present invention provides a method for enhancing cell wall properties in a plant or tree. The method comprises introducing into the plant, tree or a portion of the plant or tree, at least one nucleotide construct comprising a nucleic acid molecule operatively linked to a regulatory region active in the plant, wherein said nucleic acid molecule encodes a polypeptide with galactinol synthase (GolS)-like activity and growing or growing the plant under conditions that permit the expression of the nucleic acid, thereby enhancing the cell wall property of the plant.

Enhanced cell wall property may comprise increased cell wall density, increased wood density, reduced microfibril angle, increased tension wood formation, increased cellulose content, altered cell wall crystallinity, reduced lignin content, modified hemicellulose matrix, modified pectin matrix or a combination thereof.

Plants, perennial plants or trees that exhibit one or more enhanced cell wall property and/or altered level of cell wall traits and/or altered level of carbohydrates may be grown used as feedstock for biofuel production derived from lignocellulosic material using methods that are know to one of skill in the art. Furthermore, the plants, perennial plants or trees that exhibit one or more enhanced cell wall property and/or altered level of carbohydrates may be grown and used for pulp wood, chemical cellulose and lumber production. In addition the plants, perennial plants or trees having one or more enhanced cell wall property and/or altered level of carbohydrates may be grown and used as food stuff for life stock.

Furthermore, the present invention provides a method for altering level of carbohydrates in a plant, perennial plant or tree or portion thereof. The method comprises introducing into the plant, tree or a portion of the plant or tree, at least one nucleotide construct comprising a nucleic acid molecule operatively linked to a regulatory region active in the plant, wherein said nucleic acid molecule encodes a polypeptide with galactinol synthase (GolS)-like activity and incubating or growing the plant under conditions that permit the expression of the nucleic acid, thereby altering the level of carbohydrates in the plant. Altered level of carbohydrates may comprise an increase of total hexose, a decrease in pentose or a combination thereof. Furthermore, the level of galactose and/or glucose may increased and/or the level of xylose may decreased in a plant over-expressing a polypeptide with galactinol synthase (GolS)-like activity.

In the method of the present invention, the cell wall density may increase from about 2 to about 100%, when compared to the same parameter determined of a plant of the same species, grown under the same conditions and wherein the plant is not transformed with a nucleic acid molecule encodes a polypeptide with galactinol synthase (GolS)-like activity. Furthermore, in the method of the present invention, microfibril angle may be decreased from about 2 to about 40%, when compared to the same parameter determined of a plant of the same species, grown under the same conditions and wherein the plant is not transformed with a nucleic acid molecule encodes a polypeptide with galactinol synthase (GolS)-like activity. In the method of the present invention wood density may be increased from about 2 to about 100%, when compared to the same parameter determined of a plant of the same species, grown under the same conditions and wherein the plant is not transformed with a nucleic acid molecule encodes a polypeptide with galactinol synthase (GolS)-like activity. In addition, in the method of the current invention wood density may be increased from about 2 to about 50%, when compared to the same parameter determined of the plant of the same species, grown under the same conditions and wherein the woody plant or tree is not transformed with a nucleic acid molecule encodes a polypeptide with galactinol synthase (GolS)-like activity. Furthermore, in the method of the present invention, cellulose content may be increased from about 2 to about 50%, when compared to the same parameter determined of the plant of the same species, grown under the same conditions and wherein the plant is not transformed with a nucleic acid molecule encodes a polypeptide with galactinol synthase (GolS)-like activity. In the method of the present invention, crystallinity may be altered from about 2 to about 50%, when compared to the same parameter determined in the plant of the same species, grown under the same conditions and wherein the plant is not transformed with a nucleic acid molecule encodes a polypeptide with galactinol synthase (GolS)-like activity. In the present invention, lignin content may be further decreased from about 2 to about 50%, when compared to the same parameter determined in the plant of the same species, grown under the same conditions and wherein the plant is not transformed with a nucleic acid molecule encodes a polypeptide with galactinol synthase (GolS)-like activity.

In the current method lignin monomer composition may be altered such for example the ratio between syringyl to guaiacyl, or the p-hydroxybenzoate composition may be altered.

A method for producing a feedstock for use in pulp and paper, chemical cellulose or biofuel production comprising, providing a perennial plant comprising at least one nucleotide construct comprising a nucleic acid molecule operatively linked to a regulatory region active in the perennial plant, wherein said nucleic acid molecule encodes a polypeptide with galactinol synthase (GolS)-like activity; and growing the perennial plant under conditions that permit the expression of the nucleic acid, thereby producing the feedstock.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 shows the cDNA sequence of At1g09350 (SEQ ID NO.1)

FIG. 2 shows the protein sequence of At1g09350 (SEQ ID NO.2)

FIG. 3 shows the genomic sequence of At1g09350 (SEQ ID NO.3)

FIG. 4A shows the protein sequence of A. thaliana GolS 2 (SEQ ID NO.4)

FIG. 4B shows the mRNA sequence of A. thaliana GolS 2 (SEQ ID NO.5)

FIG. 5A shows the protein sequence of A. thaliana GolS 1 (SEQ ID NO. 6)

FIG. 5B shows A. thaliana GolS 1 mRNA sequence (SEQ ID NO.7)

FIG. 6A shows AY126715.1 (Glycine max galactinol synthase mRNA) (SEQ ID NO. 12). 6B shows AY379783.1 (Cucurbita pepo galactinol synthase (GAS1) gene (SEQ ID NO. 13). 6C shows a CLUSTAL W (1.81) Multiple Sequence Alignments of AtGolS3 (SEQ ID NO. 1), Glycine_max_AY126715 (SEQ ID NO. 12) and Cucurbita_pepo_AY379783 (SEQ ID NO. 13). 6D shows 3AtGolS3 (SEQ ID No. 2). 6E shows Glycine max GolS (SEQ ID NO 8). 6F shows Cucurbita pepo sequence (SEQ ID NO. 9). 6G shows a CLUSTAL W (1.81) Multiple Sequence Alignments of AtGolS3 (SEQ ID NO. 2), Glycine_max_AY126715 (SEQ ID NO. 8) and Cucurbita_pepo_AY379783 (SEQ ID NO. 9).

FIG. 7A shows GolS3 galactinol synthase 3. 7B shows a BLAST search of At1g09350 (SEQ ID No. 1).

FIG. 8 shows a schematic for the biosynthesis of galactinol, raffinose, and stachyose in plants.

FIG. 9 shows a graph with relative expression 2̂ (−ΔCt) of the At GolS3 in phloem of hybrid poplar.

FIGS. 10A and 10B shows a graph with relative expression 2̂ (−ΔCt) of the At GolS3 in 4 tissues in hybrid poplar. Transcript amount of the Arabidopsis thaliana galactinol synthase 3 gene (AtGolS3) in tissues of five months old greenhouse-grown hybrid poplar presented as expression relative to the transcription initiation factor 5A (TIFSA=reference gene) using the formula 2̂ (−ΔCt).

FIGS. 11A and 11B shows a graph with soluble galactinol in galactinol synthase overexpressing transgenic poplar compared to wild-type wild-type poplar in selected tissues of five-month old greenhouse-grown wild-type wild-type and AtGolS3 transgenic hybrid poplar. The concentration of galactinol in selected tissue of five-month old greenhouse-grown wild-type wild-type and AtGolS3 transgenic hybrid poplar is shown.

FIGS. 12A and 12B shows a graph with soluble myo-inositol in galactinol synthase overexpressing transgenic poplar compared to wild-type wild-type poplar in selected tissues of five-month old greenhouse-grown wild-type wild-type and AtGolS3 transgenic hybrid poplar. The concentration of myo-inositol in selected tissue of five-month old greenhouse-grown wild-type wild-type and AtGolS3 transgenic hybrid poplar is shown.

FIGS. 13A and 13B shows a graph with soluble raffinose in galactinol synthase overexpressing transgenic poplar compared to wild-type wild-type poplar in selected tissues of five-month old greenhouse-grown wild-type wild-type and AtGolS3 transgenic hybrid poplar. The concentration of raffinose in selected tissue of five-month old greenhouse-grown wild-type wild-type and AtGolS3 transgenic hybrid poplar is shown.

FIGS. 14A and 14B shows a graph with soluble sucrose in galactinol synthase overexpressing transgenic poplar compared to wild-type wild-type poplar in selected tissues of five-month old greenhouse-grown wild-type wild-type and AtGolS3 transgenic hybrid poplar. The concentration of sucrose in selected tissue of five-month old greenhouse-grown wild-type wild-type and AtGolS3 transgenic hybrid poplar is shown.

FIG. 15 shows a neighbor-joining trees constructed using the predicted amino acid sequences of confirmed and putative galactinol synthases. 15A) and 15C) GolS from Populus grouped in four clades: a, b, c, and d, and 15B) and 15D) GolS from different plant species. Phytozome accession numbers are provided for the P. trichocarpa GolS genes and GenBank accession number are provided for the remaining plants. Phylogenetic analyses suggested a putative role for hybrid poplar enzymes during biotic or abiotic stress.

FIGS. 16 A-16U show sequence of SEQ ID NO. 16-37.

FIG. 17 shows five months old greenhouse-grown transgenic poplar trees expressing the Arabidopsis thaliana galactinol synthase 3 gene (AtGolS3) and wild-type wild-type.

FIG. 18 shows a graph with the height from the base of the stem to the apex, and diameter at 20 cm from the base of the stem of three-month old greenhouse-grown hybrid poplar.

FIG. 19 shows auto-florescence (A-C) and calcofluor (D-F) staining of wild-type (A, D), AtGolS3 transgenic line 6 (B, E) and transgenic line 11 (C, F) hybrid poplar. Transgenic lines show an increased cellulose staining with calcofluor. (Scale bars: 70 μm).

FIG. 20 shows immunofluorescence labeling of xylem tissue from wild-type wild-type (A, D and G); AtGolS3 transgenic line 6 (B, E and H) and AtGolS3 transgenic line 11 (C, F and I) hybrid poplar. Tissue was label with the anti-xylan LM10 antibody (A-C); the anti-RGI CCRCM7 antibody (D-F) and the anti-mannan antibody (G-I).

FIG. 21 shows HSQC 2D-NMR of cell wall lignin of greenhouse-grown wild-type wild-type and AtGolS3 transgenic hybrid poplar.

FIG. 22 shows HSQC 2D-NMR of cell wall Polysaccharide anomeric region of greenhouse-grown wild-type wild-type and AtGolS3 transgenic hybrid poplar.

FIG. 23 shows fiber length and width of five-month old greenhouse-grown wild-type wild-type and AtGolS3 transgenic hybrid poplar.

FIG. 24 shows deduced amino acid sequence alignment of known GolS proteins fromArabidopsis thaliana (AtGolS1 and -5), Ajuga reptans (ArGolS1 and -2), Oryza sativa (OsGolS1), Cucumis melo (CmGolS1) and the two GolS proteins isolated from the P. alba×grandidentata hybrid poplar (Pa×gGolSI and Pa×gGolS11). The predicted protein sequences of both isoforms (Pa×gGolSI and Pa×gGolSII) showed characteristics of galactinol synthases from other species, including a serine phosphorylation site at position 274 and the pentapeptide hydrophobic domain ASAAP.

### DETAILED DESCRIPTION

This invention relates to enhancing cell wall properties in plants or trees. More specifically, the invention relates to the expression of an enzyme with galactinol synthase (GolS), or GolS-like, activity in plants or trees to enhance cell wall properties in the plant or tree.

The present invention provides compositions and methods for enhancing cell wall properties in plant tissues or cells, such as for example woody angiosperm and gymnosperm, by manipulating the production of raffinose family of oligosaccharides (RFO). As described in more detail below, overexpression of a polynucleotide sequence encoding an enzyme with galactinol synthase (GolS) or GolS-like activity in a plant or tree, or portions thereof, enhances one or more cell wall properties of the plant or tree. Examples of one or more cell wall properties that may be enhanced arising from the ectopic expression of GolS include, but are not limited to, an increase in cell wall density, an increase in specific gravity, a reduction in microfibril angle, an increase in tension wood formation, an increase in cellulose content, altered cellulose crystallinity, a decrease in lignin content, an altered lignin monomer composition as for example an altered syringyl to guaiacyl ratio, a modified hemicellulose and pectin matrix, or a combination thereof, of a cell, tissue, organ of a plant or tree, when compared to the same or similar cell, tissue, organ of a plant or tree in which GolS is not over expressed.

Galactinol synthase (GolS) is also known as inositol 3-α-galactosyltransferase, UDP-D-galactose:inositol galactosyltransferase; UDP-galactose:myo-inositol 1-α-D-galactosyltransferase; UDPgalactose: myo-inositol 1-α-D-galactosyltransferase; galactinol synthase; inositol 1-α-galactosyltransferase; and uridine diphosphogalactose-inositol galactosyltransferase (Enzyme database number EC 2.4.1.123). GolS catalyzes the first step in the biosynthesis of RFOs, by reversibly synthesizing galactinol from UDP-D-galactose and myo-inositol (see for example FIG. 8). Galactinol is as a substrate for the formation of the larger soluble oligosaccharides as for example raffinose, stachyose and verbascose.

A polypeptide is said to have GolS-like activity when it has one or more of the properties of the native protein, for example, synthesizing galactinol from UDP-D-galactose and myo-inositol. It is within the skill in the art to assay protein activities obtained from various sources to determine whether the properties of the proteins are the same. In so doing, one of skill in the art may employ any of a wide array of known assays including, for example, biochemical assays. For example, one of skill in the art could readily produce a plant transformed with a GolS polypeptide variant and assay a property of native GolS protein in that plant material to determine whether a particular GolS property was retained by the variant.

Accordingly, the present invention relates to methods and compositions for enhancing cell wall properties in plant tissues or cells or tree tissues or cells, such as for example woody angiosperm and gymnosperm cells, by modifying the activity of GolS or a GolS-like enzyme. The method involves introducing a nucleic acid sequence encoding GolS or an enzyme exhibiting GolS-like activity into plant or tree cells or whole plants or tress, and expressing the nucleic acid sequence in the plant or tree cells, thereby enhancing cell wall properties of the plant or tree.

The present invention also provides a method for producing a feedstock for use in pulp and paper, chemical cellulose, solid lumber or biofuel production comprising, providing a perennial plant comprising at least one nucleotide construct comprising a nucleic acid molecule operatively linked to a regulatory region active in the perennial plant, wherein said nucleic acid molecule encodes a polypeptide with galactinol synthase (GolS)-like activity; and growing the perennial plant under conditions that permit the expression of the nucleic acid, thereby producing the feedstock.

The present invention further relates to compositions comprising nucleic acid molecules comprising sequences encoding plant GolS or a GolS-like and the polypeptides encoded thereby. These sequences may be used alone, or in combination with other sequences, for example but not limited to sucrose synthase (Coleman et al., 2009, PNAS) and lignin biosynthetic genes or transcription factors that regulate lignification such as for example ferulate-5-hydroxylase (Humphreys, Hemm and Chapple, 2006 PNAS; Franke et al., 2000 Plant Journal; Huntley et al., 2003 Journal of Agriculture and Food Chemistry), can be used to enhance cell wall properties. The present invention also includes nucleic acids, expression cassettes and transformation vectors comprising the GolS or a GolS-like nucleotide sequences. The transformation vectors can be used to transform plants and express the polypeptide enhancing the cell wall properties of the transformed cells. Transformed cells as well as regenerated transgenic plants, trees, or portions thereof, and seeds comprising and expressing the GolS or a GolS-like DNA sequences and protein products are also provided.

Nucleic acid sequences encoding GolS isolated from Arabidopsis thaliana were obtained (SEQ ID NOS: 1, 5 and 7). The corresponding amino acids sequences are provided as SEQ ID NOS: 2, 4, and 6. In addition, GolS nucleic acid sequences were isolated from Poplar (SEQ ID NOS: 26-37). The corresponding amino acid sequences are provided as SEQ ID NOS: 16-25.

Therefore, the nucleic acid may comprise a nucleotide sequence encoding a polypeptide with galactinol synthase (GolS)-like activity operatively linked to a regulatory region active in a plant, wherein the polypeptide is encoded for example by the sequence of SEQ ID NOS: 2, 4, 6, 16-25, 38-45.

Specific sequences referred to in the present invention, may be considered similar to a specific sequence, based on sequence alignment. Sequences are similar when at least about 70%, or 70-100%, or any amount therebetween, for example, 70, 72, 74, 76, 78, 80, 82, 84, 86, 88, 90, 92, 94, 96, 98, 100%, or any amount therebetween, of the nucleotides of the sequences match over a defined length of the nucleotide sequence, and encode a product that exhibits GolS activity (synthesize galactinol from UDP-D-galactose and myo-inositol). Such a sequence similarity may be determined using a nucleotide sequence comparison program, such as that provided within DNASIS (using, for example but not limited to, the following parameters: GAP penalty 5, #of top diagonals 5, fixed GAP penalty 10, k-tuple 2, floating gap 10, and window size 5). However, other methods of alignment of sequences for comparison are well-known in the art for example the algorithms of Smith & Waterman (1981, Adv. Appl. Math. 2:482), Needleman & Wunsch (J. Mol. Biol. 48:443, 1970), Pearson & Lipman (1988, Proc. Nat'l. Acad. Sci. USA 85:2444), and by computerized implementations of these algorithms (GAP, BESTFIT, FASTA, and BLAST, available through the NIH), or by manual alignment and visual inspection (see, e.g., Current Protocols in Molecular Biology, Ausubel et al., eds. 1995 supplement), or using Southern or Northern hybridization under stringent conditions (see Maniatis et al., in Molecular Cloning (A Laboratory Manual), Cold Spring Harbor Laboratory, 1982). Preferably, sequences that are substantially homologous exhibit at least about 80% and most preferably at least about 90% sequence similarity over a defined length of the molecule.

Nucleotide sequence that hybridize under stringent hybridisation conditions to a complement of a nucleotide sequence encoding GolS may also be considered similar provided that the sequence encodes a product that exhibits GolS activity (synthesize galactinol from UDP-D-galactose and myo-inositol). Hybridization under stringent hybridization conditions is known in the art (see for example Current Protocols in Molecular Biology, Ausubel et al., eds. 1995 and supplements; Maniatis et al., in Molecular Cloning (A Laboratory Manual), Cold Spring Harbor Laboratory, 1982; Sambrook and Russell, in Molecular Cloning: A Laboratory Manual, 3^{rd} edition 2001; each of which is incorporated herein by reference). An example of one such stringent hybridization conditions may be about 16-20 hours hybridization in 4×SSC at 65° C., followed by washing in 0.1×SSC at 65° C. for an hour, or 2 washes in 0.1×SSC at 65° C. each for 20 or 30 minutes. Alternatively, an exemplary stringent hybridization condition could be overnight (16-20 hours) in 50% formamide, 4×SSC at 42° C., followed by washing in 0.1×SSC at 65° C. for an hour, or 2 washes in 0.1×SSC at 65° C. each for 20 or 30 minutes, or overnight (16-20 hours), or hybridization in Church aqueous phosphate buffer (7% SDS; 0.5M NaPO₄ buffer pH 7.2; 10 mM EDTA) at 65° C., with 2 washes either at 50° C. in 0.1×SSC, 0.1% SDS for 20 or 30 minutes each, or 2 washes at 65° C. in 2×SSC, 0.1% SDS for 20 or 30 minutes each.

Nucleic acid fragments encoding at least a portion of several GolS can be isolated and identified by comparison of random plant cDNA sequences to public databases containing nucleotide and protein sequences using the BLAST algorithms well known to those skilled in the art. The nucleic acid fragments of the present invention may be used to isolate cDNAs and sequences encoding homologous proteins from the same or other plant species. Isolation of homologous sequences using sequence-dependent protocols is well known in the art. Examples of sequence-dependent protocols include, but are not limited to, methods of nucleic acid hybridization, and methods of DNA and RNA amplification as exemplified by various uses of nucleic acid amplification technologies (e.g., polymerase chain reaction, ligase chain reaction).

Nucleic acid sequences encoding other GolS, either as cDNAs or genomic DNA sequences, may be isolated directly by using all or a portion of the nucleic acid fragments described herein. The nucleic acid sequences described herein may be used as hybridization probes to screen libraries from any desired plant or tree employing methodology well known to those skilled in the art. Specific oligonucleotide probes based upon nucleic acid sequences can be designed and synthesized by methods known in the art (Maniatis et al., in Molecular Cloning (A Laboratory Manual). Moreover, an entire sequence can be used directly to synthesize DNA probes by methods known to the skilled artisan such as random primer DNA labeling, nick translation, end-labeling techniques, or RNA probes using available in vitro transcription systems. In addition, specific primers can be designed and used to amplify a part or all of the instant sequences. The resulting amplification products can be labeled directly during amplification reactions or labeled after amplification reactions, and used as probes to isolate full length cDNA or genomic fragments under conditions of appropriate stringency.

Non limiting examples of nucleic acid or amino acid sequences that might be suitable for the present invention are listed in Table 1 and in the sequence listing. Homologues of GolS are also described for example in U.S. Pat. Nos. 7,294,756 and 7,476,778 (which are herein incorporated by reference in their entirety).

TABLE 1

| Accession | |
|---|---|
| Number | Description |
| NP_172406.1 | galactinol synthase 3 [Arabidopsis thaliana] >gb\|AAK48973.1\|AF370546_1 water stress induced protein-like protein |
| | [Arabidopsis thaliana] >gb\|AAC33195.1\| Similar to rice water stress induced protein gi\|537404 [Arabidopsis thaliana] |
| | >dbj\|BAB78532.1\| galactinol synthase [Arabidopsis thaliana] >gb\|AAM10014.1\| similar to rice water stress induced protein |
| | [Arabidopsis thaliana] >gb\|AEE28432.1\| galactinol synthase 3 [Arabidopsis thaliana] |
| XP_002889744.1 | ATGOLS3 [Arabidopsis lyrata subsp. lyrata] >gb\|EFH66003.1\| ATGOLS3 [Arabidopsis lyrata subsp. lyrata] |
| XP_002891985.1 | hypothetical protein ARALYDRAFT_474819 [Arabidopsis lyrata subsp. lyrata] >gb\|EFH68244.1\| hypothetical protein |
| | ARALYDRAFT_474819 |
| | [Arabidopsis lyrata subsp. lyrata] |
| NP_176053.1 | galactinol synthase 2 [Arabidopsis thaliana] >gb\|AAG09103.1\|AC009323_14 Putative galactinol synthase [Arabidopsis thaliana] |
| | >gb\|AAK91426.1\| At1g56600/F25P12_16 |
| | [Arabidopsis thaliana] >gb\|AAL15412.1\| At1g56600/F25P12_16 [Arabidopsis thaliana] >dbj\|BAB78531.1\| galactinol synthase |
| | [Arabidopsis thaliana] >gb\|AEE33413.1\| galactinol synthase 2 [Arabidopsis thaliana] |
| AAM19710.1 | galactinol synthase-like protein [Eutrema halophilum] |
| XP_002882112.1 | ATGOLS1 [Arabidopsis lyrata subsp. lyrata] >gb\|EFH58371.1\| ATGOLS1 [Arabidopsis lyrata subsp. lyrata] |
| XP_002512546.1 | conserved hypothetical protein [Ricinus communis] >gb\|EEF49998.1\| conserved hypothetical protein [Ricinus communis] |
| NP_182240.1 | galactinol synthase 1 [Arabidopsis thaliana] >gb\|AAB63818.1\| putative galactinol synthase [Arabidopsis thaliana] |
| | >gb\|AAL07218.1\| putative galactinol synthase |
| | [Arabidopsis thaliana] >dbj\|BAB78530.1\| galactinol synthase [Arabidopsis thaliana] >gb\|AAM15468.1\| putative galactinol synthase |
| | [Arabidopsis thaliana] >gb\|AAM14365.1\| putative galactinol synthase [Arabidopsis thaliana] >gb\|AAM61564.1\| putative galactinol |
| | synthase [Arabidopsis thaliana] >gb\|AEC10811.1\| galactinol synthase 1 [Arabidopsis thaliana] |
| XP_002320958.1 | predicted protein [Populus trichocarpa] >gb\|EEE99273.1\| predicted protein [Populus trichocarpa] |
| ADG03603.1 | galactinol synthase [Brassica napus] |
| ACJ15472.1 | galactinol synthase [Brassica napus] |
| AAD26116.1 | galactinol synthase [Brassica napus] |
| XP_002515233.1 | conserved hypothetical protein [Ricinus communis] >gb\|EEF47217.1\| conserved hypothetical protein [Ricinus communis] |
| XP_002512547.1 | conserved hypothetical protein [Ricinus communis] >gb\|EEF49999.1\| conserved hypothetical protein [Ricinus communis] |
| ADM92589.1 | galactinol synthase [Coffea arabica] |
| BAH98060.1 | galactinol synthase [Solanum lycopersicum] |
| ABQ44212.1 | galactinol synthase [Capsicum annuum] |
| XP_002301531.1 | predicted protein [Populus trichocarpa] >gb\|EEE80804.1\| predicted protein [Populus trichocarpa] |
| ACA04033.1 | galactinol synthase 4 [Populus trichocarpa x Populus deltoides] |
| CAN79630.1 | hypothetical protein VITISV_039943 [Vitis vinifera] |
| XP_002279114.1 | PREDICTED: hypothetical protein isoform 1 [Vitis vinifera] |
| ACA04032.1 | galactinol synthase 3 [Populus trichocarpa] |
| XP_002319473.1 | predicted protein [Populus trichocarpa] >gb\|EEE95396.1\| predicted protein [Populus trichocarpa] |
| CAB51130.1 | putative galactinol synthase [Pisum sativum] |
| XP_002281369.1 | PREDICTED: hypothetical protein [Vitis vinifera] >emb\|CBI39626.3\| unnamed protein product [Vitis vinifera] |
| CAN74708.1 | hypothetical protein VITISV_018010 [Vitis vinifera] |
| XP_002319472.1 | predicted protein [Populus trichocarpa] >gb\|EEE95395.1\| predicted protein [Populus trichocarpa] |
| XP_002281304.1 | PREDICTED: hypothetical protein [Vitis vinifera] |
| ACI62176.1 | galactinol synthase [Boea hygrometrica] |
| ABK27907.1 | galactinol synthase [Xerophyta viscosa] |
| AAO84915.1 | galactinol synthase [Cucumis sativus] |
| ACT34765.1 | galactinol synthase [Salvia miltiorrhiza] |
| XP_002314975.1 | predicted protein [Populus trichocarpa] >gb\|EEF01146.1\| predicted protein [Populus trichocarpa] |
| AAL78687.1 | galactinol synthase [Cucumis melo] |
| CAN66209.1 | hypothetical protein VITISV_035072 [Vitis vinifera] |
| ACF88041.1 | unknown [Zea mays] >gb\|ACG34552.1\| galactinol synthase 3 [Zea mays] |
| NP_001105748.1 | galactinol synthase 1 [Zea mays] >gb\|AAQ07248.1\|AF497507_1 galactinol synthase 1 [Zea mays] |
| XP_002519283.1 | conserved hypothetical protein [Ricinus communis] >gb\|EEF43147.1\| conserved hypothetical protein [Ricinus communis] |
| AAM96867.1 | galactinol synthase [Glycine max] |
| ADM92590.1 | galactinol synthase [Coffea arabica] |
| XP_002281261.1 | PREDICTED: hypothetical protein [Vitis vinifera] |
| ABF66656.1 | galactinol synthase [Ammopiptanthus mongolicus] >gb\|ABU41005.1\| galactinol synthase [Ammopiptanthus mongolicus] |
| XP_002280616.1 | PREDICTED: hypothetical protein [Vitis vinifera] |
| ADF43063.1 | galactinol synthase [Ammopiptanthus nanus] |
| NP_001060697.1 | Os07g0687900 [Oryza sativa Japonica Group] >dbj\|BAA05538.1\| WS176 protein induced by water stress |
| | [Oryza sativa Japonica Group] >dbj\|BAC21346.1\| WSI76 protein induced by water stress [Oryza sativa Japonica Group] |
| | >dbj\|BAD30298.1\| WSI76 protein induced by water stress [Oryza sativa Japonica Group] > |
| | dbj\|BAF22611.1\| Os07g0687900 [Oryza sativa Japonica Group] >dbj\|BAG94187.1\| unnamed protein product |
| | [Oryza sativa Japonica Group] >dbj\|BAG97931.1\| unnamed protein product [Oryza sativa Japonica Group] |
| XP_002279157.1 | PREDICTED: hypothetical protein isoform 1 [Vitis vinifera] |
| NP_001049939.1 | Os03g0316200 [Oryza sativa Japonica Group] >gb\|ABF95621.1\| galactinol synthase 3, putative, expressed |
| | [Oryza sativa Japonica Group] >dbj\|BAF11853.1\| Os03g0316200 [Oryza sativa Japonica Group] >dbj\|BAG92707.1\| |
| | unnamed protein product [Oryza sativa Japonica Group] |
| EAZ05203.1 | hypothetical protein OsI_27402 [Oryza sativa Indica Group] |
| NP_176250.1 | galactinol synthase 4 [Arabidopsis thaliana] >gb\|AAB71970.1\| nearly identical to rice water stress induced protein gp |
| | \|D26537\|537404 [Arabidopsis thaliana] >gb\|AAY56403.1\| At1g60470 [Arabidopsis thaliana] >gb\|AEE33692.1\| |
| | galactinol synthase 4 [Arabidopsis thaliana] |
| XP_002262651.1 | PREDICTED: hypothetical protein isoform 1 [Vitis vinifera] |
| EAY89768.1 | hypothetical protein OsI_11309 [Oryza sativa Indica Group] |
| CAB51533.1 | galactinol synthase, isoform GolS-1 [Ajuga reptans] |
| XP_002330017.1 | predicted protein [Populus trichocarpa] >gb\|EEF08573.1\| predicted protein [Populus trichocarpa] |
| BAE99313.1 | hypothetical protein [Arabidopsis thaliana] |
| XP_002467954.1 | hypothetical protein SORBIDRAFT_01g037090 [Sorghum bicolor] >gb\|EER94952.1\| |
| | hypothetical protein SORBIDRAFT_01g037090 [Sorghum bicolor] |
| XP_002886616.1 | ATGOLS4 [Arabidopsis lyrata subsp. lyrata] >gb\|EFH62875.1\| ATGOLS4 [Arabidopsis lyrata subsp. lyrata] |
| XP_002265947.1 | PREDICTED: hypothetical protein [Vitis vinifera] |
| NP_001105750.1 | LOC606405 [Zea mays] >gb\|AAQ07250.1\|AF497509_1 galactinol synthase 3 [Zea mays] >gb\|AAO48782.1\| galactinol |
| | synthase 3 [Zea mays] |
| BAF51566.1 | galactinol synthase [Triticum aestivum] |
| BAF99254.1 | galactinol synthase [Coptis japonica] |
| BAF51565.1 | galactinol synthase [Triticum aestivum] |
| ACM50915.1 | galactinol synthase [Medicago sativa subsp. falcata] |
| ACG39512.1 | galactinol synthase 3 [Zea mays] |
| AAM97493.1 | galactinol synthase [Medicago sativa] |
| ADM92588.1 | galactinol synthase [Coffea arabica] |
| ABQ12641.1 | galactinol synthase 2 [Verbascum phoeniceum] |
| XP_002525364.1 | conserved hypothetical protein [Ricinus communis] >gb\|EEF37002.1\| conserved hypothetical protein [Ricinus communis] |
| AAL78686.1 | galactinol synthase [Cucumis melo] |
| ABK23436.1 | unknown [Picea sitchensis] >gb\|ABK23557.1\| unknown [Picea sitchensis] >gb\|ACN40280.1\| |
| | unknown [Picea sitchensis] >gb\|ACN40473.1\| unknown [Picea sitchensis] |
| XP_002312306.1 | predicted protein [Populus trichocarpa] >gb\|EEE89673.1\| predicted protein [Populus trichocarpa] |
| AAM96868.1 | fagopyritol synthase 2 [Fagopyrum esculentum] |
| ADN33831.1 | galactinol synthase [Cucumis melo subsp. melo] |
| ACA04028.1 | galactinol synthase 1 [Populus trichocarpa x Populus deltoides] |
| XP_002279136.1 | PREDICTED: hypothetical protein isoform 2 [Vitis vinifera] |
| XP_002311774.1 | predicted protein [Populus trichocarpa] >gb\|ABK93271.1\| unknown [Populus trichocarpa] >gb\|ABK95881.1\| |
| | unknown [Populus trichocarpa] >gb\|ACA04027.1\| galactinol synthase 1 [Populus trichocarpa] >gb\|EEE89141.1\| |
| | predicted protein [Populus trichocarpa] |
| ABR17981.1 | unknown [Picea sitchensis] |
| ACA04029.1 | galactinol synthase 1 [Populus trichocarpa x Populus deltoides] |
| | unknown [Populus trichocarpa] >gb\|ABK94534.1\| unknown [Populus trichocarpa] >gb\|ACA04030.1\| |
| ABK93731.1 | galactinol synthase 2 [Populus trichocarpa] |
| XP_002314613.1 | predicted protein [Populus trichocarpa] >gb\|EEF00784.1\| predicted protein [Populus trichocarpa] |
| ACA04031.1 | galactinol synthase 2 [Populus trichocarpa x Populus deltoides] |
| ABQ12640.1 | galactinol synthase 1 [Verbascum phoeniceum] |
| AAM96870.1 | fagopyritol synthase 1 [Fagopyrum esculentum] |
| XP_0024612.42.1 | hypothetical protein SORBIDRAFT_02g043450 [Sorghum bicolor] >gb\|EER97763.1\| |
| | hypothetical protein SORBIDRAFT_02g043450 [Sorghum bicolor] |
| ABK22947.1 | unknown [Picea sitchensis] |
| ABK22810.1 | unknown [Picea sitchensis] |
| XP_002262705.1 | PREDICTED: hypothetical protein isoform 2 [Vitis vinifera] |
| XP_002279176.1 | PREDICTED: hypothetical protein isoform 2 [Vitis vinifera] |
| NP_567741.2 | galactinol synthase 6 [Arabidopsis thaliana] >gb\|AAN13051.1\| galactinol synthase |
| | [Arabidopsis thaliana] >gb\|AEE85175.1\| galactinol synthase 6 [Arabidopsis thaliana] |
| XP_002869619.1 | ATGOLS6 [Arabidopsis lyrata subsp. lyrata] >gb\|EFH45878.1\| ATGOLS6 [Arabidopsis lyrata subsp. lyrata] |
| CAB51534.1 | galactinol synthase, isoform GolS-2 [Ajuga reptans] |
| NP_001105749.1 | galactinol synthase2 [Zea mays] >gb\|AAQ07249.1\|AF497508_1 galactinol synthase 2 [Zea mays] |
| NP_197768.1 | galactinol synthase 5 [Arabidopsis thaliana] >dbj\|BAB10052.1\| galactinol synthase [Arabidopsis thaliana] |
| | >gb\|AED93213.1\| galactinol synthase 5 [Arabidopsis thaliana] |
| CAB38954.1 | putative protein [Arabidopsis thaliana] >emb\|CAB79480.1\| putative protein [Arabidopsis thaliana] |
| XP_002886618.1 | predicted protein [Arabidopsis lyrata subsp. lyrata] >gb\|EFH62877.1\| predicted protein [Arabidopsis lyrata subsp. lyrata] |
| XP_002874149.1 | predicted protein [Arabidopsis lyrata subsp. lyrata] >gb\|EFH50408.1\| predicted protein [Arabidopsis lyrata subsp. lyrata] |
| EAZ26716.1 | hypothetical protein OsJ_10624 [Oryza sativa Japonica Group] |
| NP_176248.1 | galactinol synthase 7 [Arabidopsis thaliana] >gb\|AAY78659.1\| putative galactinol synthase [Arabidopsis thaliana] |
| | >gb\|AEE33688.1\| galactinol synthase 7 [Arabidopsis thaliana] |
| NP_850902.1 | putative galactinol synthase [Arabidopsis thaliana] >gb\|AED93876.1\| putative galactinol synthase [Arabidopsis thaliana] |
| CAN66320.1 | hypothetical protein VITISV_040624 [Vitis vinifera] |
| AAC24075.1 | Strong similarity to water stress-induced protein, WSI76 isolog T08I13.2 gb\|2275196 from A. thaliana BAC gb\|AC002337 |
| | [Arabidopsis thaliana] |

Lignin, inherent in the plant secondary cell wall, negatively impacts pulping and chemical pulp production and significantly impedes enzymatic accessibility (Chang & Holtzapple, 2000 Applied Biochemistry and Biotechnology 84-86: 5-37.) and competitively binds cellulolytic enzymes (Berlin et al., 2005 Applied Biochemistry and Biotechnology 121: 163-170). This underpins the innate recalcitrance of wood-derived lignocellulosic feedstocks for production of liquid biofuels. Therefore, the innate plant cell wall structure and biochemistry are key determinants of the utility of lignocellulosic feedstocks for biofuel applications. Similarly, maximizing woody biomass productivity is also critical for minimizing feedstock costs. Thus, technological advances improving growth rates and/or the design of plant cell wall biochemistries that are more amenable to conversion to fermentable sugars for bioethanol production may have a substantial impact on the overall efficacy of the bioenergy process.

By controlling or modifying the activity of a polynucleotide encoding an enzyme with GolS or GolS-like activity, the cell wall property of the plant or tree, tissue or cell may be enhanced. Modifying the activity of GolS may involve over (ectopic) expression of the nucleic acid encoding GolS, and producing an increased amount of GolS enzyme in a cell, when compared to a cell obtained from the same or similar plant or tree that has not been subject to over expression of the GolS nucleic acid. The GolS sequence may be expressed in a tissue dependent manner, for example the expression of GolS may be regulated by a tissue-specific promoter and expression localized in a desired tissue, for example, the stem, xylem, phloem, cambium, root or a combination thereof, using promoters that specifically drive the expression of the GolS sequence within one or more of these tissues. GolS may be also expressed throughout the plant or tree through the use of a ubiquitous promoter as are known in the art.

In some embodiments, the expression of the enzyme with GolS or GolS-like activity may be increased in the range from about 0.1 to about 5 relative expression 2̂ (−ΔCt), such as 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 36, 3.7, 3.8, 3.9, 4, 4.1, 4.2, 4.3, 4.4, 4.5, 4.6, 4.7, 4.8, 4.9 relative expression 2̂ (−ΔCt) or any value therebetween. The enzyme with GolS or GolS-like activity may be expressed in the phloem in the range of about 0.1 to about 2.5 relative expression 2̂ (−ΔCt), such as 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, relative expression 2̂ (−ΔCt) or any value therebetween. In one embodiment the relative expression of the enzyme with GolS or GolS-like activity in the phloem is in the range of about 0.6 to about 1 relative expression 2̂ (−ΔCt) or any value therebetween.

The enzyme with GolS or GolS-like activity may be expressed in the cambium in the range of about 0.1 to about 1.5 relative expression 2̂ (−ΔCt), such as 0.2, 0.3, 0.4, 0.5, 0.6, 0.7, 0.8, 0.9, 1, 1.1, 1.2, 1.3, 1.4, 1.5, relative expression 2̂ (−ΔCt) or any value therebetween. In one embodiment the relative expression of the enzyme with GolS or GolS-like activity in the cambium is in the range of about 0.1 to about 0.3 relative expression 2̂ (−ΔCt) or any value therebetween.

The enzyme with GolS or GolS-like activity may be expressed in the source leaf in the range of about 0.1 to about 5 relative expression 2̂ (−ΔCt), such as 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 36, 3.7, 3.8, 3.9, 4, 4.1, 4.2, 4.3, 4.4, 4.5, 4.6, 4.7, 4.8, 4.9 relative expression 2̂ (−ΔCt) or any value therebetween. In one embodiment the relative expression of the enzyme with GolS or GolS-like activity in the source leaf is in the range of about 2.2 to about 5 relative expression 2̂ (−ΔCt) or any value therebetween.

The enzyme with GolS or GolS-like activity may be expressed in the sink leaf in the range of about 1 to about 3 relative expression 2̂ (−ΔCt), such as 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3 relative expression 2̂ (−ΔCt) or any value therebetween. In one embodiment the relative expression of the enzyme with GolS or GolS-like activity in the sink leaf is between about 1.3 and about 3 relative expression 2̂ (−ΔCt) or any value therebetween.

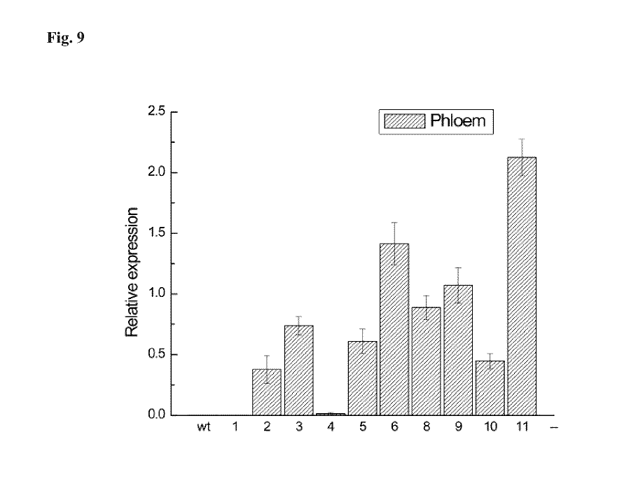

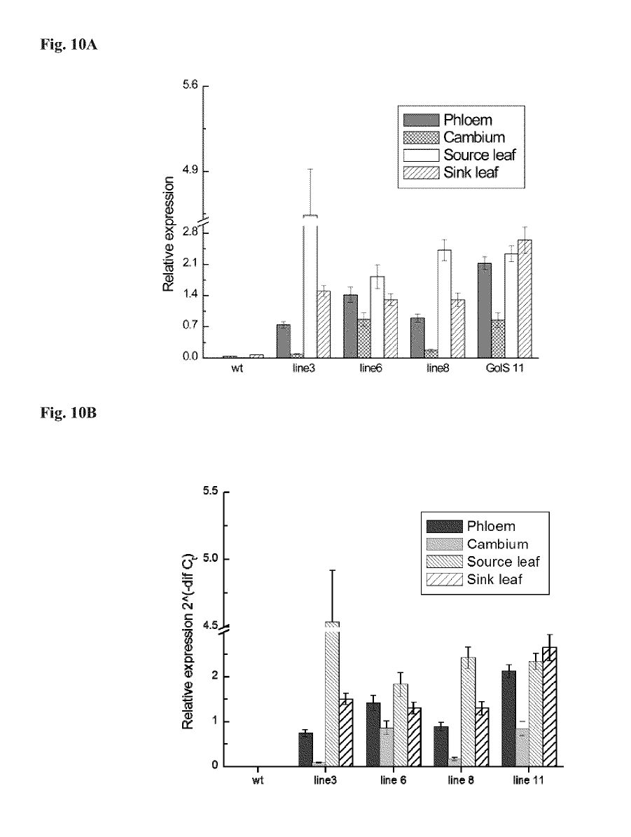

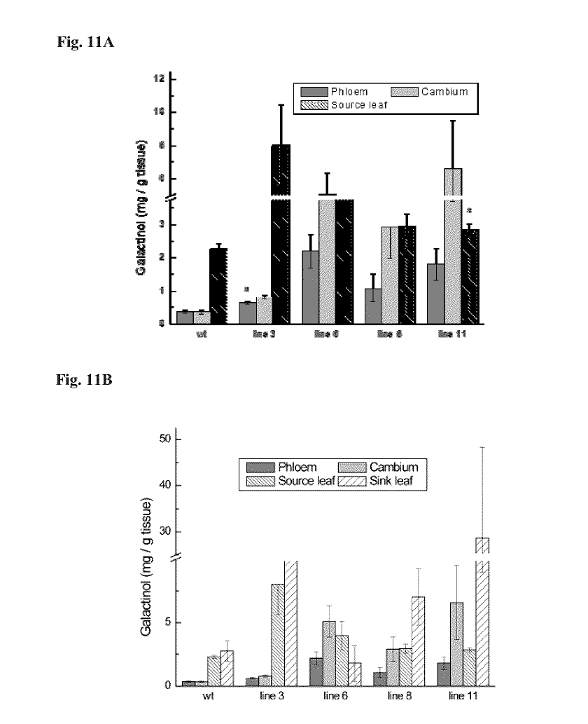

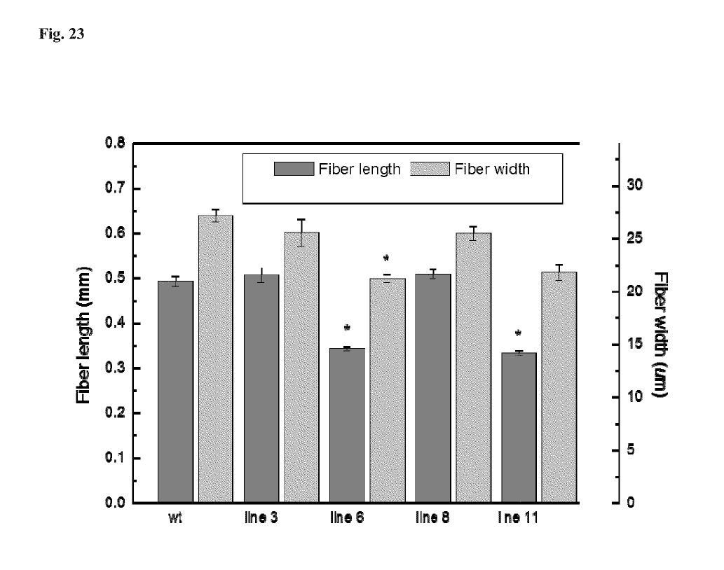

Stunted growth might be observe in transgenic plants that have a relative expression of 1.5 or higher in the phloem or 1 or higher in the cambium, when compared with the wild-type (see FIGS. 10B, 17 and 18). These plants (lines 6 and 11) show reduced fiber length and width (see FIG. 23). These plants may be used in paper production where shorter fiber length and/or fiber width might be desired, for example in the production of toilet paper, handkerchiefs, feminine hygiene products, paper towels or facial tissue.

By “enhanced cell wall property”, “enhancement of a cell wall property” or “enhancing cell wall property” it is meant a change in, but not limited to, one or more than one of the following properties: an increase in cell wall density, a decrease of microfibril angle, an increase in wood density, an increase in tension wood formation, an increase in cellulose content, an altered cell wall crystallinity, a decrease in lignin content, an altered lignin monomer composition such as for example an altered syringyl to guaiacyl ratio, a modification of hemicellulose, modification of the pectin matrix, or a combination thereof, when compared to the same parameter determined of a plant of the same species, grown under the same conditions and wherein the plant is not transformed with a nucleic acid molecule encodes a polypeptide with galactinol synthase (GolS)-like activity.

Plants or trees that exhibit one or more enhanced cell wall property may be grown used as a feedstock for biofuel production derived from lignocellulosic material using methods that are know to one of skill in the art. Furthermore, the plants or trees that exhibit one or more enhanced cell wall property may be grown and used for pulp wood production, chemical cellulose and as solid lumber. Without wishing to be bound by theory a combination of higher density and lower microfibril angle may improve the flexural properties of solid wood. Furthermore, wood with decrease lignin content, or increased cellulose content and/or altered cell wall crystallinity may be pulped in less time normally required and yielded higher quality cellulose. Manfield et al. (New Phytologist (2012) 194: 91-101) have shown that lignin quantity and quality influences production of liquid biofuels. Therefore, by controlling or modifying the activity of a polynucleotide encoding an enzyme with GolS or GolS-like activity, biosynthesis of key cell wall constituents, can positively impact the production of ethanol from feedstocks that have traditionally been regarded as highly recalcitrant.

Chen & Dixon 2007 (Nature Biotechnology 25: 759-761), showed that plants with lower lignin contents were more amenable to hydrolysis. The extent of total cell wall lignin content also positively impacted the efficiency of the steam explosion process, and other known processes for example dilute acid, hot water, AFEX, organosolvent and ionic liquid applications, as shown in other potential energy crops, such as grain (Mussatto et al., 2008 Enzyme and Microbial Technology 43: 124-129), corn stover (Oehgren et al., 2007, Bioresource Technology 98: 2503-2510), sugarcane (Martin et al., 2007, Enzyme and Microbial Technology 40: 426-432) in which lower lignin content improved the subsequent hydrolysis of the wood residue and fermentation processes. Without wishing to be bound by theory there may be a relationship between decreasing lignin content and the ability for cellulolytic enzymes to access the remaining wall carbohydrate matrices.

Using the methods as described herein, a plant, portion of a plant or plant cell with an increase in cell wall density, a decrease of microfibril angle, an increase in wood density, an increase in tension wood formation, an increase in cellulose content, an altered cell wall crystallinity, a decrease in lignin content, an altered lignin monomer composition such as for example an altered syringyl to guaiacyl ratio, a modification of hemicellulose, modification of the pectin matrix, or a combination thereof, may be obtained when compared to the same parameter determined of a plant of the same species, grown under the same conditions and wherein the plant is not transformed with a nucleic acid molecule encodes a polypeptide with galactinol synthase (GolS)-like activity.

By an increase in cellulose content, it is meant an increase by about 2% to about 100%, or any amount therebetween as determined using standard techniques in the art, for example, from about 10% to about 50% or any value therebetween for example about 2, 5, 8, 10, 12, 15, 18, 20, 22, 24, 25, 26, 28, 30, 32, 34, 35, 36, 38, 40, 42, 44, 45, 46, 48, 50, 52, 54, 55, 56, 58, 60, 65, 70, 75, 80, 85, 90, 95, or 100%, when compared to the same parameter determined in a plant, woody plant or tree of the same species, grown under the same conditions and in the absence of ectopic expression of the enzyme with GolS or GolS-like active in the plant.

By cell wall density it is meant the mass or weight per unit volume of cell wall. By increased cell wall density it is meant an increase or enhancement of cell wall density by about 2 to about 50% or any amount therebetween, for example from about 4, 6, 8, 10, 12, 14, 16, 18, 20, 22, 24, 26, 28, 30, 32, 34, 36, 38, 40, 42, 44, 46, 48, 50%, when compared to the same parameter determined of a plant, perennial plant, woody plant or tree of the same species, grown under the same conditions and wherein the plant, woody plant or tree is not transformed with a nucleic acid molecule encodes a polypeptide with galactinol synthase (GolS)-like activity.

By microfibril angle, is meant the angle between the direction of the helical windings of cellulose microfibrils in the secondary cell wall of fibres and tracheids and the long axis of cell. It is usually applied to the orientation of cellulose microfibrils in the S2 layer that makes up the greatest proportion of the secondary cell wall thickness. Using the methods as described herein, microfibril angle in plants, perennial plants, woody plants or trees expressing the polypeptide with GolS-like activity may decrease by at least about 2 to about 40% or any amount therebetween, for example from about 4, 6, 8, 10, 12, 14, 16, 18, 20, 22, 24, 26, 28, 30, 32, 34, 36, 38, or 40%, or any amount therebetween, compared to microfibril angle in plants, perennial plants, woody plants of trees of the same species grown under similar conditions and wherein the plant woody plants of trees is not transformed with a nucleic acid molecule encodes a polypeptide with galactinol synthase (GolS)-like activity.

By wood density or specific gravity, it is meant the density of oven-dry wood relative to the density of water. The specific gravity of wood gives a measure of the amount of wood substance present in a sample. Using the methods as described herein, the wood density (specific gravity) of wood cells expressing the polypeptide with GolS-like activity may increase by at least about 2 to about 100% or any amount therebetween, for example from about 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98%, or any amount therebetween, when compared to the wood density (specific gravity) of a plant of the same species grown under similar conditions and wherein the plant is not transformed with a nucleic acid molecule encodes a polypeptide with galactinol synthase (GolS)-like activity.

By the term tension wood it is meant a type of reaction wood that when compared to normal wood comprises a higher cellulose content, altered cell wall crystallinity (cellulose crystallinity), lower microfibril angle a lower lignin content, an altered lignin monomer composition such for example an altered syringyl to guaiacyl ratio, and a higher density. Using the methods as described herein, tension wood formation in perennial plants, woody plants or trees expressing the polypeptide with GolS-like activity may increase by at least about 2 to about 50% or any amount therebetween, for example from about 4, 6, 8, 10, 12, 14, 16, 18, 20, 22, 24, 26, 28, 30, 32, 34, 36, 38, 40, 42, 44, 46, 48, 50%, or any amount therebetween, compared to tension wood formation in perennial plants, woody plants or trees of the same species grown under similar conditions and wherein the plant is not transformed with a nucleic acid molecule encodes a polypeptide with galactinol synthase (GolS)-like activity.

By a decrease in lignin content, it is meant a decrease by about 2% to about 100%, or any amount therebetween, for example, from about 10% to about 50% or any value therebetween for example about 2, 5, 8, 10, 12, 15, 18, 20, 22, 24, 25, 26, 28, 30, 32, 34, 35, 36, 38, 40, 42, 44, 45, 46, 48, 50, 52, 54, 55, 56, 58, 60, 65, 70, 75, 80, 85, 90, 95, or 100% when compared to the same parameter determined of a plant, perennial plants, woody plant or tree of the same species, grown under the same conditions and wherein the plant, woody plant or tree is not transformed with a nucleic acid molecule encodes a polypeptide with galactinol synthase (GolS)-like activity.

Using the methods as described herein, the total cellulose content of a plant, perennial plant, woody plant or tree cell expressing the polypeptide with GolS-like activity may increase by at least about 2 to about 50% or any amount therebetween, for example from about 4, 6, 8, 10, 12, 14, 16, 18, 20, 22, 24, 26, 28, 30, 32, 34, 36, 38, 40, 42, 44, 46, 48, 50%, or any amount therebetween, compared to the total cellulose content of a plant, perennial plant, woody plant or tree of the same species grown under similar conditions and wherein the plant, perennial plant, woody plant or tree is not transformed with a nucleic acid molecule encodes a polypeptide with galactinol synthase (GolS)-like activity. Additionally, the total lignin content of a plant, perennial plant, woody plant or tree cell expressing the polypeptide with GolS-like activity may decrease by at least about 2 to about 50% or any amount therebetween, for example from about 4, 6, 8, 10, 12, 14, 16, 18, 20, 22, 24, 26, 28, 30, 32, 34, 36, 38, 40, 42, 44, 46, 48, 50%, or any amount therebetween, when compared to the lignin content of a plant, perennial plant, woody plant or tree of the same species grown under similar conditions and wherein the plant, perennial plant, woody plant or tree is not transformed with a nucleic acid molecule encodes a polypeptide with galactinol synthase (GolS)-like activity.

Using the methods as described herein, the total cellulose crystallinity of a plant cell expressing the polypeptide with GolS-like activity may increase by at least about 2 to about 50% or any amount therebetween, for example from about 4, 6, 8, 10, 12, 14, 16, 18, 20, 22, 24, 26, 28, 30, 32, 34, 36, 38, 40, 42, 44, 46, 48, 50%, or any amount therebetween, compared to the total cellulose crystallinity content of a plant of the same species grown under similar conditions and wherein the plant is not transformed with a nucleic acid molecule encodes a polypeptide with galactinol synthase (GolS)-like activity

Lignocellulosic materials are potential sources of sugars for ethanol production. The separation of lignin from the cellulose and hemicellulose to make the material susceptible to hydrolysis. The hydrolysis of cellulose and hemicellulose takes place at different rates and prolonged reaction can degrade the sugars into materials that are not suitable for ethanol production. Furthermore, the hydrolysis of these materials produces a variety of sugars. Not all of these sugars are currently fermentable with the standard yeast strains that are used in the ethanol industry. The pentose sugars are particularly difficult to ferment.

Using the method as described herein levels of carbohydrates in a plant, perennial plant or tree may be altered. The method comprises introducing into the plant, perennial plant, tree or a portion of the plant, perennial plant or tree, at least one nucleotide construct comprising a nucleic acid molecule operatively linked to a regulatory region active in the plant, wherein said nucleic acid molecule encodes a polypeptide with galactinol synthase (GolS)-like activity and growing the plant under conditions that permit the expression of the nucleic acid, thereby altering the level of carbohydrates in the plant. Altered level of carbohydrates may comprise an increase of total hexose, a decrease in pentose or a combination thereof. Furthermore, the level of galactose and/or glucose may increased and/or the level of xylose may decreased in a plant over-expressing a polypeptide with galactinol synthase (GolS)-like activity.

Using the methods as described herein, the total hexose content of a plant cell expressing the polypeptide with GolS-like activity may increase by at least about 2 to about 50% or any amount therebetween, for example from about 4, 6, 8, 10, 12, 14, 16, 18, 20, 22, 24, 26, 28, 30, 32, 34, 36, 38, 40, 42, 44, 46, 48, 50%, or any amount therebetween, compared to the total hexose content of a plant of the same species grown under similar conditions and wherein the plant is not transformed with a nucleic acid molecule encodes a polypeptide with galactinol synthase (GolS)-like activity.

Furthermore, using the methods as described herein, the total pentose content of a plant cell expressing the polypeptide with GolS-like activity may decrease by at least about 2 to about 50% or any amount therebetween, for example from about 4, 6, 8, 10, 12, 14, 16, 18, 20, 22, 24, 26, 28, 30, 32, 34, 36, 38, 40, 42, 44, 46, 48, 50%, or any amount therebetween, compared to the total pentose content of a plant of the same species grown under similar conditions and wherein the plant is not transformed with a nucleic acid molecule encodes a polypeptide with galactinol synthase (GolS)-like activity.

By modification of hemicellulose it is meant to alter the absolute composition of the monomeric constituents such as for example arabinose, galactose, glucose, xylose, or mannose, to alter the degree of branching or sidechains adhering to the carbohydrate backbones, or a combination thereof. By modification of the pectin matrix it is meant to alter the absolute composition of the monomeric constituents such as for example rhamnose, galactose, fucose, galacturonic acid, arabinose, or methylated forms thereof, to alter the degree of branching or sidechains adhering to the carbohydrate backbones or a combination thereof.

Furthermore using the methods as described herein, the total galactose content of a plant cell expressing the polypeptide with GolS-like activity may increase by at least about 2% to about 100% or any amount therebetween, for example from about 15, 20, 25, 30, 35, 40, 45, 50, 55, 60, 65, 70, 75, 80, 85, 90, 95, 100%, or any amount therebetween, compared to the total galactose content of a plant of the same species grown under similar conditions and wherein the plant is not transformed with a nucleic acid molecule encodes a polypeptide with galactinol synthase (GolS)-like activity.

Additionally, the total glucose content of a plant cell expressing the polypeptide with GolS-like activity may increase by at least about 10 to about 50% or any amount therebetween, for example from about 12, 14, 16, 18, 20, 22, 24, 26, 28, 30, 32, 34, 36, 38, 40, 42, 44, 46, 48, 50%, or any amount therebetween, when compared to the glucose content of a plant of the same species grown under similar conditions and wherein the plant is not transformed with a nucleic acid molecule encodes a polypeptide with galactinol synthase (GolS)-like activity.

Furthermore, using the methods as described herein, the total xylose content of a woody plant or tree cell expressing the polypeptide with GolS-like activity may decrease by at least 2 to about 50% or any amount therebetween, for example from about 4, 6, 8, 10, 12, 14, 16, 18, 20, 22, 24, 26, 28, 30, 32, 34, 36, 38, 40, 42, 44, 46, 48, 50% or any amount therebetween, compared to the total xylose content of a perennial plant, woody plant or tree of the same species grown under similar conditions and wherein the woody plant or tree is not transformed with a nucleic acid molecule encodes a polypeptide with galactinol synthase (GolS)-like activity.

Furthermore, using the methods as described herein, the total mannose content of a plant expressing the polypeptide with GolS-like activity may decrease by at least 2 to about 50% or any amount therebetween, for example from about 4, 6, 8, 10, 12, 14, 16, 18, 20, 22, 24, 26, 28, 30, 32, 34, 36, 38, 40, 42, 44, 46, 48, 50% or any amount therebetween, compared to the total mannose content of a plant of the same species grown under similar conditions and wherein the plant is not transformed with a nucleic acid molecule encodes a polypeptide with galactinol synthase (GolS)-like activity.

Using the methods as described herein, the total galactose content of a plant expressing the polypeptide with GolS-like activity may increase by at least 2 to about 50% or any amount therebetween, for example from about 4, 6, 8, 10, 12, 14, 16, 18, 20, 22, 24, 26, 28, 30, 32, 34, 36, 38, 40, 42, 44, 46, 48, 50% or any amount therebetween, compared to the total galactose content of a woody plant or tree of the same species grown under similar conditions and wherein the plant is not transformed with a nucleic acid molecule encodes a polypeptide with galactinol synthase (GolS)-like activity.

Additionally, using the methods as described herein, the total arabinose content of plant cell expressing the polypeptide with GolS-like activity may increase by at least 2 to about 50% or any amount therebetween, for example from about 4, 6, 8, 10, 12, 14, 16, 18, 20, 22, 24, 26, 28, 30, 32, 34, 36, 38, 40, 42, 44, 46, 48, 50% or any amount therebetween, compared to the total arabinose content of a plant of the same species grown under similar conditions and wherein the plant is not transformed with a nucleic acid molecule encodes a polypeptide with galactinol synthase (GolS)-like activity.

Using the methods as described herein, the total galactinol concentration of a plant cell expressing the polypeptide with GolS-like activity may increase by at least 20 to about 2200% or any amount therebetween, for example from about 40, 60, 80, 100, 120, 140, 160, 180, 200, 220, 240, 260, 280, 300, 320, 340, 360, 380, 400, 450, 500, 50, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200% or any amount therebetween, compared to the total galactinol content of a plant of the same species grown under similar conditions and wherein the plant is not transformed with a nucleic acid molecule encodes a polypeptide with galactinol synthase (GolS)-like activity.

Furthermore, using the methods as described herein, the total myo-inositol content of a plant cell expressing the polypeptide with GolS-like activity may increase by at least 20 to about 1200% or any amount therebetween, for example from about 40, 60, 80, 100, 120, 140, 160, 180, 200, 220, 240, 260, 280, 300, 320, 340, 360, 380, 400, 450, 500, 50, 600, 650, 700, 750, 800, 850, 900, 950, 1000, 1050, 1100, 1150, 1200% or any amount therebetween, compared to the total myo-inositol content of a plant of the same species grown under similar conditions and wherein the plant is not transformed with a nucleic acid molecule encodes a polypeptide with galactinol synthase (GolS)-like activity.

Using the methods as described herein, the total raffinose content of a plant cell expressing the polypeptide with GolS-like activity may increase by at least 50 to about 6000% or any amount therebetween, for example from about 40, 60, 80, 100, 120, 140, 160, 180, 200, 220, 240, 260, 280, 300, 320, 340, 360, 380, 400, 450, 500, 50, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 42800, 4900, 5000, 5100, 5200, 5300, 5400, 5500, 5600, 5700, 5800, 5900, 6000% or any amount therebetween, compared to the total raffinose content of a plant of the same species grown under similar conditions and wherein the plant is not transformed with a nucleic acid molecule encodes a polypeptide with galactinol synthase (GolS)-like activity.

In addition, using the methods as described herein, the total sucrose content of a plant cell expressing the polypeptide with GolS-like activity may increase by at least 5 to about 1200% or any amount therebetween, for example from about 10, 20, 30, 40, 60, 80, 100, 120, 140, 160, 180, 200, 220, 240, 260, 280, 300, 320, 340, 360, 380, 400, 450, 500, 50, 600, 650, 700, 750, 800, 850, 900, 950, 1000, 1100, 1200% or any amount therebetween, compared to the total raffinose content of a plant of the same species grown under similar conditions and wherein the plant is not transformed with a nucleic acid molecule encodes a polypeptide with galactinol synthase (GolS)-like activity.

The invention further provides vectors comprising a nucleic acid molecule encoding a polypeptide with galactinol synthase (GolS)-like activity. The vectors of the invention may also contain termination sequences, which are positioned downstream of the nucleic acid molecules of the invention, such that transcription of mRNA is terminated, and polyA sequences added. Exemplary of such terminators are the cauliflower mosaic virus (CaMV) 35S terminator and the nopaline synthase gene (NOS) terminator. The expression vector also may contain enhancers, start codons, splicing signal sequences, and targeting sequences.

Expression vectors of the invention may also contain a selection marker by which transformed cells can be identified in culture. The marker may be associated with the heterologous nucleic acid molecule, i.e., the gene operably linked to a promoter. As used herein, the term “marker” refers to a gene encoding a trait or a phenotype that permits the selection of, or the screening for, a plant or cell containing the marker. In plants, for example, the marker gene will encode antibiotic or herbicide resistance. This allows for selection of transformed cells from among cells that are not transformed or transfected.

In the context of this disclosure, the term “regulatory element” or “regulatory region” typically refers to a sequence of DNA, usually, but not always, upstream (5′) to the coding sequence of a structural gene, which controls the expression of the coding region by providing the recognition for RNA polymerase and/or other factors required for transcription to start at a particular site. However, it is to be understood that other nucleotide sequences, located within introns, or 3′ of the sequence may also contribute to the regulation of expression of a coding region of interest. An example of a regulatory element that provides for the recognition for RNA polymerase or other transcriptional factors to ensure initiation at a particular site is a promoter element. Most, but not all, eukaryotic promoter elements contain a TATA box, a conserved nucleic acid sequence comprised of adenosine and thymidine nucleotide base pairs usually situated approximately 25 base pairs upstream of a transcriptional start site. A promoter element comprises a basal promoter element, responsible for the initiation of transcription, as well as other regulatory elements (as listed above) that modify gene expression.

There are several types of regulatory regions, including those that are developmentally regulated, tissue specific, inducible or constitutive. A regulatory region that is developmentally regulated, or controls the differential expression of a gene under its control, is activated within certain organs or tissues of an organ at specific times during the development of that organ or tissue. However, some regulatory regions that are developmentally regulated may preferentially be active within certain organs or tissues at specific developmental stages, they may also be active in a developmentally regulated manner, or at a basal level in other organs or tissues within the plant as well. Examples of tissue-specific regulatory regions, for example see-specific a regulatory region, include but are not limited to stem-specific promoters (Bam P. et. al. 2008, Proc S Afr Sug Technology Ass 81:508-512), a xylem-specific promoter (Lu H., et. al. 2003, Plant Growth Regulation 3:279-286). An example of a leaf-specific promoter includes the plastocyanin promoter (see U.S. Pat. No. 7,125,978, which is incorporated herein by reference).

An inducible regulatory region is one that is capable of directly or indirectly activating transcription of one or more DNA sequences or genes in response to an inducer. In the absence of an inducer the DNA sequences or genes will not be transcribed. Typically the protein factor that binds specifically to an inducible regulatory region to activate transcription may be present in an inactive form, which is then directly or indirectly converted to the active form by the inducer. However, the protein factor may also be absent. The inducer can be a chemical agent such as a protein, metabolite, growth regulator, herbicide or phenolic compound or a physiological stress imposed directly by heat, cold, salt, or toxic elements or indirectly through the action of a pathogen or disease agent such as a virus. A plant cell containing an inducible regulatory region may be exposed to an inducer by externally applying the inducer to the cell or plant such as by spraying, watering, heating or similar methods. Inducible regulatory elements may be derived from either plant or non-plant genes (e.g. Gatz, C. and Lenk, LR. P., 1998, Trends Plant Sci. 3, 352-358; which is incorporated by reference). Examples, of potential inducible promoters include, but not limited to, tetracycline-inducible promoter (Gatz, C., 1997, Ann. Rev. Plant Physiol. Plant Mol. Biol. 48, 89-108; which is incorporated by reference), steroid inducible promoter (Aoyama. T. and Chua, N. H., 1997, Plant 1. 2, 397-404; which is incorporated by reference) and ethanol-inducible promoter (Salter, M. G., et al, 1998, Plant Journal 16, 127-132; Caddick, M. X., et al, 1998, Nature Biotech. 16, 177-180, which are incorporated by reference) cytokinin inducible IB6 and CKI 1 genes (Brandstatter, I. and Kieber, 1.1., 1998, Plant Cell 10, 1009-1019; Kakimoto, T., 1996, Science 274, 982-985; which are incorporated by reference) and the auxin inducible element, DR5 (Ulmasov, T., et al., 1997, Plant Cell 9, 1963-1971; which is incorporated by reference).

A constitutive regulatory region directs the expression of a gene throughout the various parts of a plant and continuously throughout plant development. Examples of known constitutive regulatory elements include promoters associated with the CaMV 35S transcript (Odell et al., 1985, Nature, 313: 810-812), the rice actin 1 (Zhang et al, 1991, Plant Cell, 3: 1155-1165), actin 2 (An et al., 1996, Plant J., 10: 107-121), or tms 2 (U.S. Pat. No. 5,428,147, which is incorporated herein by reference), and triosephosphate isomerase 1 (Xu et. al., 1994, Plant Physiol. 106: 459-467) genes, the maize ubiquitin 1 gene (Cornejo et al, 1993, Plant Mol. Biol. 29: 637-646), the Arabidopsis ubiquitin 1 and 6 genes (Holtorf et al, 1995, Plant Mol. Biol. 29: 637-646), and the tobacco translational initiation factor 4A gene (Mandel et al, 1995, Plant Mol. Biol. 29: 995-1004).

The term “constitutive” as used herein does not necessarily indicate that a gene under control of the constitutive regulatory region is expressed at the same level in all cell types, but that the gene is expressed in a wide range of cell types even though variation in abundance is often observed. Constitutive regulatory elements may be coupled with other sequences to further enhance the transcription and/or translation of the nucleotide sequence to which they are operatively linked. For example, the CPMV-HT system is derived from the untranslated regions of the Cowpea mosaic virus (CPMV) and demonstrates enhanced translation of the associated coding sequence.

“Promoter” connotes a region of DNA upstream from the start of transcription that is involved in recognition and binding of RNA polymerase and other proteins to initiate transcription. A “constitutive promoter” is one that is active throughout the life of the plant and under most environmental conditions. Tissue-specific, tissue-preferred, cell type-specific, and inducible promoters constitute the class of “non-constitutive promoters.

Promoters useful for expression of a nucleic acid sequence introduced into a cell to increase expression of GolS may be constitutive promoters, such as the cauliflower mosaic virus (CaMV) 35S promoter, or tissue-specific, tissue-preferred, cell type-specific, and inducible promoters. For example, by using vascular system-specific, xylem-specific, or xylem-preferred promoters, one can modify GolS activity specifically in many tissues such as vascular tissues, especially xylem (for example, cellulose synthase: CesA8 promoter). The use of a constitutive promoter in general affects enzyme levels and functions in all parts of the plant, while use of a tissue-preferred promoter permits targeting of the modified gene expression to specific plant parts, leading to a more controllable phenotypes.

By “operatively linked” it is meant that the particular sequences, for example a regulatory element and a coding region of interest, interact either directly or indirectly to carry out an intended function, such as mediation or modulation of gene expression. The interaction of operatively linked sequences may, for example, be mediated by proteins that interact with the operatively linked sequences. In general, the nucleic acid sequences that are operatively linked may be contiguous.

By “transformation” it is meant the stable interspecific transfer of genetic information (nucleotide sequence) that is manifested genotypically, phenotypically or both. The interspecific transfer of genetic information from a construct to a host may be heritable and the transfer of genetic information considered stable, or the transfer may be transient and the transfer of genetic information is not inheritable. Methods for stable transformation, and regeneration of plants are established in the art and known to one of skill in the art. The method of obtaining transformed and regenerated plants is not critical to the present invention.

The cassette or construct of the present invention can be introduced into plant cells using Ti plasmids, Ri plasmids, plant virus vectors, direct DNA transformation, micro-injection, electroporation, etc. For reviews of such techniques see for example Weissbach and Weissbach, Methods for Plant Molecular Biology, Academy Press, New York VIII, pp. 421-463 (1988); Geierson and Corey, Plant Molecular Biology, 2d Ed. (1988); and Miki and Iyer, Fundamentals of Gene Transfer in Plants. In Plant Metabolism, 2d Ed. D T. Dennis, D H Turpin, D D Lefebrve, D B Layzell (eds), Addison Wesly, Langmans Ltd. London, pp. 561-579 (1997). Other methods include direct DNA uptake, the use of liposomes, electroporation, for example using protoplasts, micro-injection, microprojectiles or whiskers, vacuum infiltration, or as described herein. See, for example, Bilang et al. (Gene 100: 247-250 (1991), Scheid et al. (Mol. Gen. Genet. 228: 104-112, 1991), Guerche et al. (Plant Science 52: 111-116, 1987), Neuhause et al. (Theor. Appl Genet. 75: 30-36, 1987), Klein et al., Nature 327: 70-73 (1987); Howell et al. (Science 208: 1265, 1980), Horsch et al. (Science 227: 1229-1231, 1985), DeBlock et al., Plant Physiology 91: 694-701, 1989), Methods for Plant Molecular Biology (Weissbach and Weissbach, eds., Academic Press Inc., 1988), Methods in Plant Molecular Biology (Schuler and Zielinski, eds., Academic Press Inc., 1989), Liu and Lomonossoff (J Virol Meth, 105:343-348, 2002,), U.S. Pat. Nos. 4,945,050; 5,036,006; and 5,100,792, U.S. patent application Ser. No. 08/438,666, filed May 10, 1995, and 07/951,715, filed Sep. 25, 1992, (all of which are hereby incorporated by reference).

To aid in identification of transformed plant cells, the constructs of this invention may be further manipulated to include plant selectable markers. Useful selectable markers include enzymes that provide for resistance to chemicals such as an antibiotic for example, gentamycin, hygromycin, kanamycin, or herbicides such as phosphinothrycin, glyphosate, chlorosulfuron, and the like. Similarly, enzymes providing for production of a compound identifiable by colour change such as GUS (beta-glucuronidase), or luminescence, such as luciferase or GFP, may be used. Examples of suitable selectable markers include adenosine deaminase, dihydrofolate reductase, hygromycin-B-phosphotransferase, thymidne kinase, xanthine-guanine phospho-ribosyltransferase, glyphosate and glufosinate resistance, and amino-glycoside 3′-O-phosphotranserase (kanamycin, neomycin and G418 resistance). These markers may include resistance to G418, hygromycin, bleomycin, kanamycin, and gentamicin. The construct may also contain the selectable marker gene Bar that confers resistance to herbicidal phosphinothricin analogs like ammonium gluphosinate. Thompson et al., EMBO J. 9: 2519-23 (1987). Other suitable selection markers are known as well.

The present invention further provides a method for modifying cell wall properties in plants or trees comprising,

i) providing the plant or tree comprising a nucleic acid sequence comprising a regulatory region operatively associated with a silencing nucleotide sequence, wherein expression of the silencing nucleotide sequence reduces or eliminates the expression of a polypeptide with galactinol synthase (GolS)-like activity, and

ii) expressing the silencing nucleotide sequence within the plant or tree, to reduce the level of the polypeptide with galactinol synthase (GolS)-like activity in the plant or tree, the reduced level of polypeptide with (GolS)-like activity may be determined by comparing the level of expression of the polypeptide with galactinol synthase (GolS)-like activity in the plant or tree, with a level of the polypeptide with galactinol synthase (GolS)-like activity in a second plant, or tree from the second plant or tree, that does not express the silencing nucleic acid sequence.

The endogenous GolS or (GolS)-like gene may be inhibited by RNAi-mediated suppression, ribozyme, antisense RNA or a transcription factor, for example, a native transcription factor, or a synthetic transcription factor. Furthermore, the GolS or (GolS)-like gene that is targeted for inhibition or silencing within the plant may be inhibited or silenced using a portion of GolS or (GolS)-like gene, for example by using a 5′, a 3′; or both 5′ and 3′ specific regions of GolS or (GolS)-like gene. Examples of 5′ or 3′ regions of GolS or (GolS)-like gene that may be used for silencing include the nucleotide sequences defined in SEQ ID NO: 26-37, a nucleotide sequence that exhibits from about 80 to about 100% sequence identity to the nucleotide sequences defined in SEQ ID NO: 26-37, a nucleotide sequence that hybridizes to the nucleotide sequence defined in SEQ ID NO: 26-37 or its complement, under stringent hybdridization conditions as defined above.

The level of the GolS activity or GolS-like activity, or the expression of the nucleotide sequence encoding a polypeptide with GolS activity or GolS-like activity, within a plant or tree may be reduced by inhibiting the expression of the polypeptide with GolS activity or GolS-like activity for example by inhibiting transcription of the gene encoding the polypeptide with GolS activity or GolS-like activity, reducing levels of the transcript, or inhibiting synthesis of the GolS or GolS-like protein. The levels of polypeptide with GolS activity or GolS-like activity may be inhibited from about 10% to about 100%, or any amount therebetween, where compared to the level of polypeptide with GolS activity or GolS-like activity obtained from a second plant that expresses the nucleotide sequence at wild-type levels. For example, the protein may be reduced by from about 10% to about 80% or any amount therebetween, about 10% to about 50% or any amount therebetween, about 10% to about 40% or any amount therebetween, from about 10% to about 30%, or any amount therebetween, about 10% to about 20% or any amount therebetween, or about 10, 15, 20, 25, 30, 35, 40, 45, 50, 55, 60, 65, 70, 76, 80, 85, 90, 95 or 100%, or any amount therebetween. Furthermore, the level of the nucleotide encoding GolS or GolS-like may be inhibited from about 10% to about 100%, or any amount therebetween, where compared to the level of the nucleotide encoding GolS activity or GolS-like activity obtained from a second plant that expresses the nucleotide sequence at wild-type levels. For example, the expression of the nucleotide sequence may be reduced by from about 10% to about 80% or any amount therebetween, about 10% to about 50% or any amount therebetween, about 10% to about 40% or any amount therebetween, from about 10% to about 30%, or any amount therebetween, about 10% to about 20% or any amount therebetween, or about 10, 15, 20, 25, 30, 35, 40, 45, 50, 55, 60, 65, 70, 76, 80, 85, 90, 95 or 100%, or any amount therebetween.

By “reduction of gene expression” or reduction of expression” it is meant the reduction in the level of mRNA, protein, or both mRNA and protein, encoded by a gene or nucleotide sequence of interest. Reduction of gene expression may arise as a result of the lack of production of full length RNA, for example mRNA, or through cleaving the mRNA, for example with a ribozyme (e.g. see Methods in Molecular Biology, vol 74 Ribozyme Protocols, P.C. Turner, ed, 1997, Humana Press), or RNAi (e.g. see Gene Silencing by RNA Interference, Technology and Application, M. Sohail ed, 2005, CRC Press; Fire A, et al, 1998, Horiguchi G, 2004; Wesley et al. 2001), or otherwise reducing the half-life of RNA, using antisense (e.g. see Antisense Technology, A Practical Approach, C. Lichtenstien and W. Nellen eds., 1997, Oxford University Press), ribozyme, RNAi techniques, or by using a natural or synthetic transcription factor that is targeted to the promoter and results in the down regulation of GolS or Gols-like expression.

A “silencing nucleotide sequence” refers to a sequence that when transcribed results in the reduction of expression of a target gene, or it may reduce the expression of two or more than two target genes, for example, 1, 2, 3, 4, 5, 6, 7, 8, 9, 10 target genes, or any number of target genes therebetween. A silencing nucleotide sequence may involve the use of antisense RNA, a ribozyme, or RNAi, targeted to a single target gene, or the use of antisense RNA, ribozyme, or RNAi, comprising two or more than two sequences that are linked or fused together and targeted to two or more than two target genes. When transcribed the product of the silencing nucleotide sequence may target one, or it may target two or more than two, of the target genes. When two or more than two sequences are linked or fused together, these sequences may be referred to as gene fusions, or gene stacking. It is within the scope of the present invention that gene fusions may comprise 2, 3, 4, 5, 6, 7, 8, 9, 10 nucleotide sequences, or any number therebetween, that are fused or linked together. The fused or linked sequences may be immediately adjacent each other, or there may be linker fragment between the sequences. Reduction in the expression of GolS or GolS-like activity, results in the reduced synthesis of a protein encoded by the GolS or GolS-like sequence.

When the GolS activity or GolS-like activity is to be preferentially reduced, a nucleotide sequence that is specific for the 5′, 3′, or both 5′ and 3′ regions of the GolS or GolS-like gene may be used. These regions of GolS or GolS-like exhibit reduced sequence homology when compared to other GolS or GolS-like genes.

In the present invention the activity of GolS activity or GolS-like activity may be selectively or preferentially inhibited. By “preferential inhibition” or “selective inhibition” it is meant that the expression of the target nucleotide sequence is inhibited by about 5 to about 100% when compared to the expression of a reference sequence. For example, the expression of the desired sequence may be inhibited by about 20 to about 80%, or any amount therebetween, or 20-50%, or any amount therebetween, when compared to the expression of the same sequence in a plant of the same variety (or genetic background) that does not express a silencing sequence, for example a wild-type plant, or when compared to the expression of a reference sequence in the same plant. For example, the expression of the desired sequence may be inhibited by about 5, 10, 20, 30, 40, 50, 60, 70, 80, 90 100% or any amount therebetween, when compared to the expression of the same sequence, in a plant of the same variety (or genetic background) that does not express a silencing sequence, for example a wild-type plant, or when compared to the expression of a reference sequence in the same plant. A non-limiting example of a desired sequence is GolS or GolS-like sequence. In this case, preferential (or selective) inhibition of GolS or GolS-like is achieved when the expression of GolS or GolS-like is inhibited by about 5, 10, 20, 30, 40, 50, 60, 70, 80, 90 100% or any amount therebetween, when compared to the expression of GolS or GolS-like, in a wild-type plant of the same genetic background.

Non-limiting examples of one or more than one silencing nucleotide sequence includes SEQ ID NO: 26-37. Additional examples of a silencing nucleotide sequence include a nucleotide sequence that is from about 80 to about 100% similar, or any amount therebetween, or 80, 85, 90, 95 or 100% similar, as determined by sequence alignment of the nucleotide sequences as defined above, to SEQ ID NO: 26-37. Alternatively, an example of a silencing nucleotide sequence includes a nucleotide sequence or that hybridizes under stringent hybridization conditions, as defined above, to SEQ ID NO: 26-37. Provided that the nucleotide sequence retains the property of silencing expression of a GolS or GolS-like gene or sequence.

Also considered part of this invention are transgenic plants or trees, plant cells or tree cells, or seeds containing the construct and vector of the present invention. Methods of regenerating whole plants from plant or tree cells are also known in the art. In general, transformed plant or tree cells are cultured in an appropriate medium, which may contain selective agents such as antibiotics, where selectable markers are used to facilitate identification of transformed plant cells. Once callus forms, shoot formation can be encouraged by employing the appropriate plant hormones in accordance with known methods and the shoots transferred to rooting medium for regeneration of plants. The plants or trees may then be used to establish repetitive generations, either from seeds or using vegetative propagation techniques. Transgenic plants or trees can also be generated without using tissue cultures.

“Plant”, “perennial plant”, “woody plant” or “tree” is a term that encompasses whole plants or trees, plant or tree organs (e.g. leaves, stems, roots, etc.), seeds, differentiated or undifferentiated plant or tree cells, and progeny of the same. Plant or tree material includes, without limitation, seeds suspension cultures, embryos, meristematic regions, callus tissues, leaves, roots, shoots, stems, fruit, gametophytes, sporophytes, pollen, and microspores. The class of plants or trees which can be used in the present invention is generally as broad as the class of higher plants amenable to genetic engineering techniques, including angiosperms, both monocotyledonous and dicotyledonous plants, as well as gymnosperms.

While any plant may be used, such as for example perennial plants the present invention contemplates plants used in the solid wood, pulp and paper, dissolving pulp industry, in the biofuel industry and plants used as feed for life stock, such for example forage. Examples include, but are not limited to Cotton, Rice, Alfalfa, Triticale, Switchgrass, Miscanthus, Sorghum or Sugar Cane. Preferably, the plants are perennial plants including but not limited to herbaceous perennials, evergreen perennials or trees, deciduous perennials or trees, and woody perennials or trees. Examples of perennials plants, include, but are not limited to angiosperm trees or gymnosperm tree. Examples include Eucalyptus species such as E. alba, E. albens, E. amygdalina, E. aromaphloia, E. baileyana, E. balladoniensis, E. bicostata, E. botryoides, E. brachyandra, E. brassiana, E. brevistylis, E. brockwayi, E. camaldulensis, E. ceracea, E. cloeziana, E. coccifera, E. cordata, E. cornuta, E. corticosa, E. crebra, E. croajingolensis, E. curtisii, E. dalrympleana, E. deglupta, E. delegatensis, E. delicata, E. diversicolor, E. diversifolia, E. dives, E. dolichocarpa, E. dundasii, E. dunnii, E. elata, E. erythrocorys, E. erythrophloia, E. eudesmoides, E. falcata, E. gamophylla, E. glaucina, E. globulus, E. globulus subsp. bicostata, E. globulus subsp. globulus, E. gongylocarpa, E. grandis, E. grandis×urophylla, E. guilfoylei, E. gunnii, E. hallii, E. houseana, E. jacksonii, E. lansdowneana, E. latisinensis, E. leucophloia, E. leucoxylon, E. lockyeri, E. lucasii, E. maidenii, E. marginata, E. megacarpa, E. melliodora, E. michaeliana, E. microcorys, E. microtheca, E. muelleriana, E. nitens, E. nitida, E. obliqua, E. obtusiflora, E. occidentalis, E. optima, E. ovata, E. pachyphylla, E. pauciflora, E. pellita, E. perriniana, E. petiolaris, E. pilularis, E. piperita, E. platyphylla, E. polyanthemos, E. populnea, E. preissiana, E. pseudo globulus, E. pulchella, E. radiata, E. radiata subsp. radiata, E. regnans, E. risdonii, E. robertsonii, E. rodwayi, E. rubida, E. rubiginosa, E. saligna, E. salmonophloia, E. scoparia, E. sieberi, E. spathulata, E. staeri, E. stoatei, E. tenuipes, E. tenuiramis, E. tereticornis, E. tetragona, E. tetrodonta, E. tindaliae, E. torquata, E. umbra, E. urophylla, E. vernicosa, E. viminalis, E. wandoo, E. wetarensis, E. willisii, E. willisii subsp. falciformis, E. willisii subsp. willisii, and E. woodwardii.

The invention also contemplates Populus species such as P. alba, P. alba×P. grandidentata, P. alba×P. tremula, P. alba×P. tremula var. glandulosa, P. alba×P. tremuloides, P. balsamifera, P. balsamifera subsp. trichocarpa, P. balsamifera subsp. trichocarpa×P. deltoides, P. ciliata, P. deltoides, P. euphratica, P. euramericana, P. kitakamiensis, P. lasiocarpa, P. laurifolia, P. maximowiczii, P. maximowiczii×P. balsamifera subsp. trichocarpa, P. nigra, P. sieboldii×P. grandidentata, P. suaveolens, P. szechuanica, P. tomentosa, P. tremula, P. tremula×P. tremuloides, P. tremuloides, P. wilsonii, P. canadensis, P. yunnanensis and Conifers as, for example, loblolly pine (Pinus taeda), slash pine (Pinus elliotii), ponderosa pine (Pinus ponderosa), radiata pine (Pinus radiata), lodgepole pine (Pinus contorta), and Monterey pine (Pinus radiata); Douglas-fir (Pseudotsuga menziesii); Western hemlock (Tsuga canadensis); White spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis).

Other plants that may be modified by the process of the invention include all flowering plants. It is understood that the word “plant” includes any plant or plant material used in the pulp and paper industry, cellulose industry or biofuel industry.

The present invention includes nucleotide sequences:

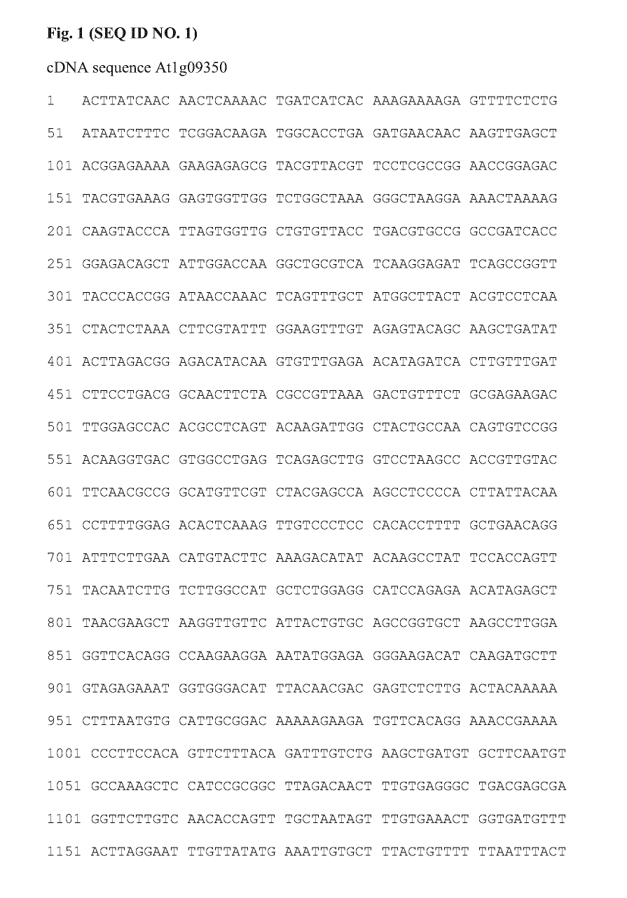

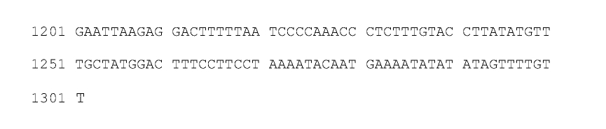

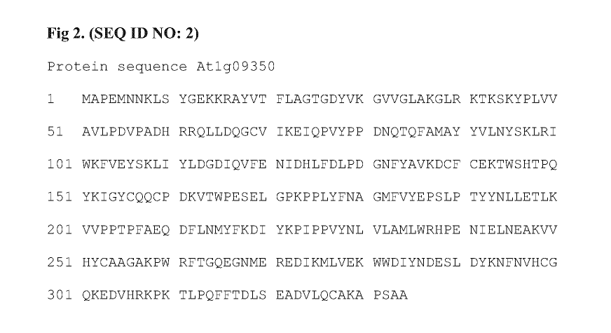

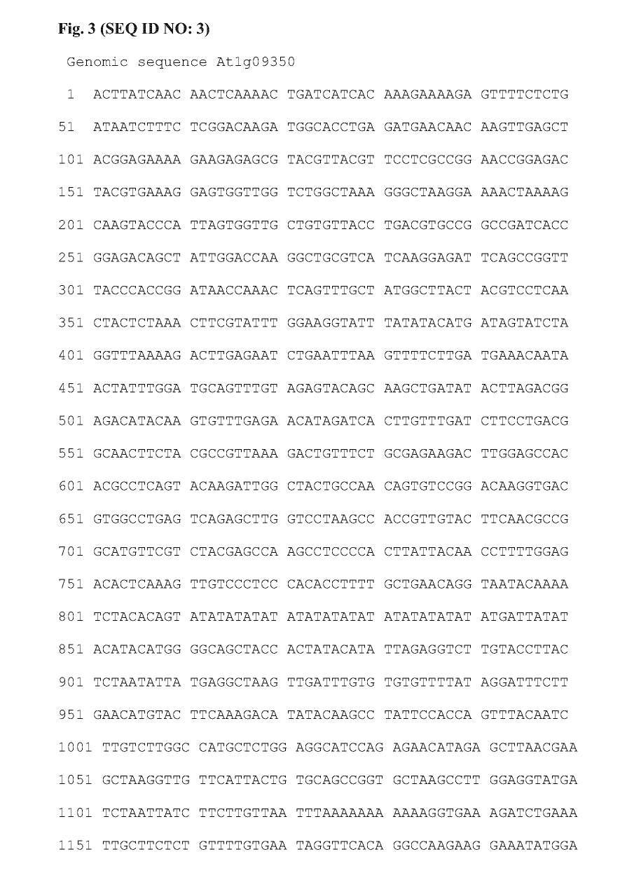

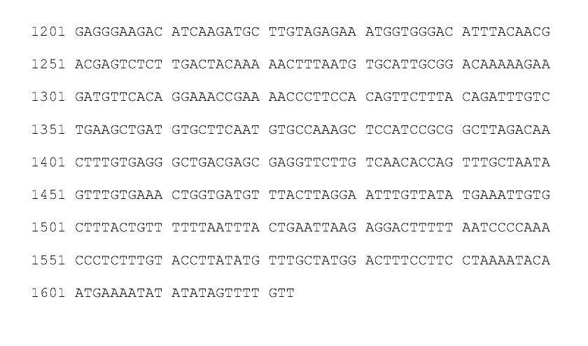

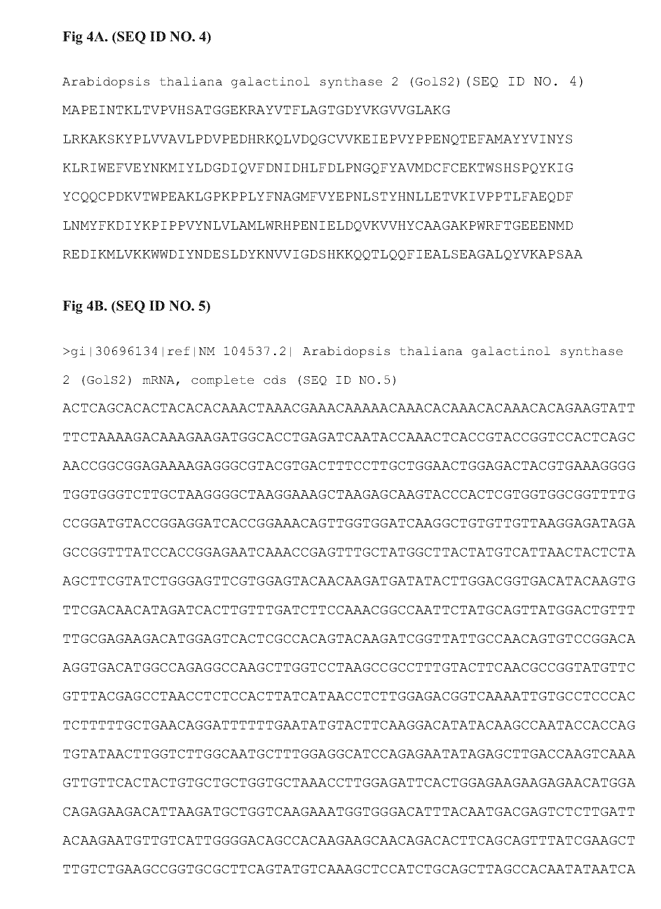

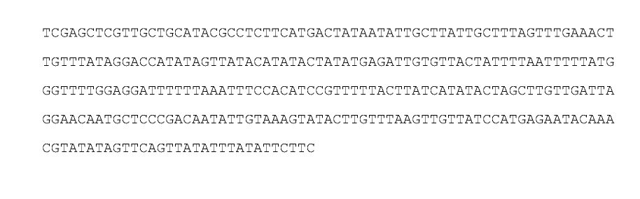

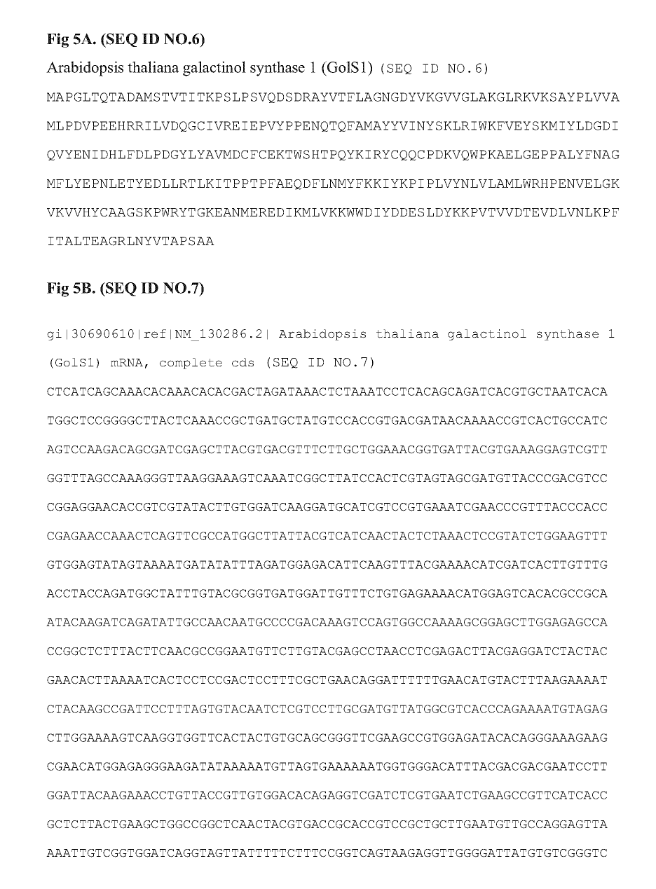

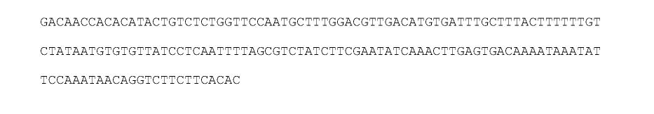

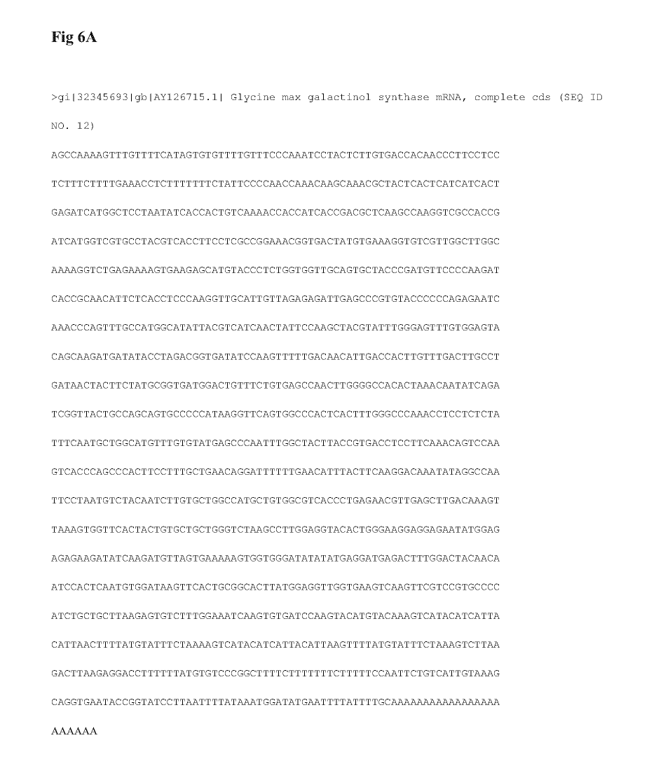

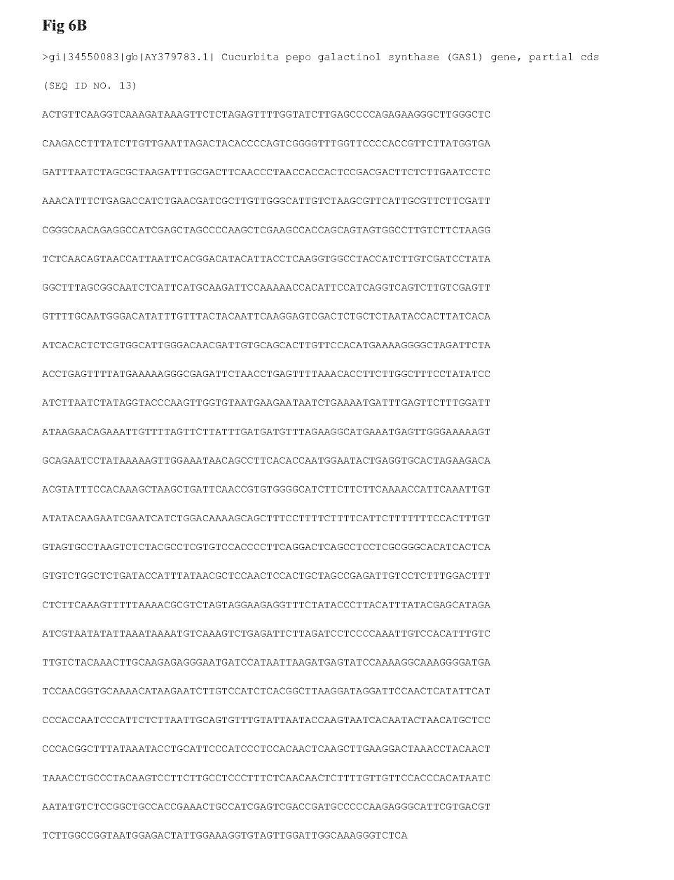

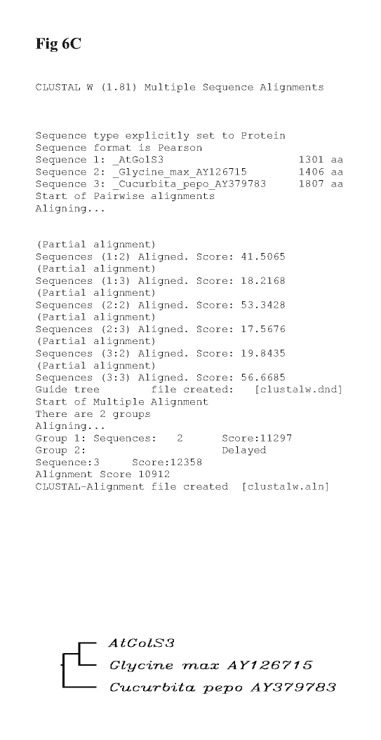

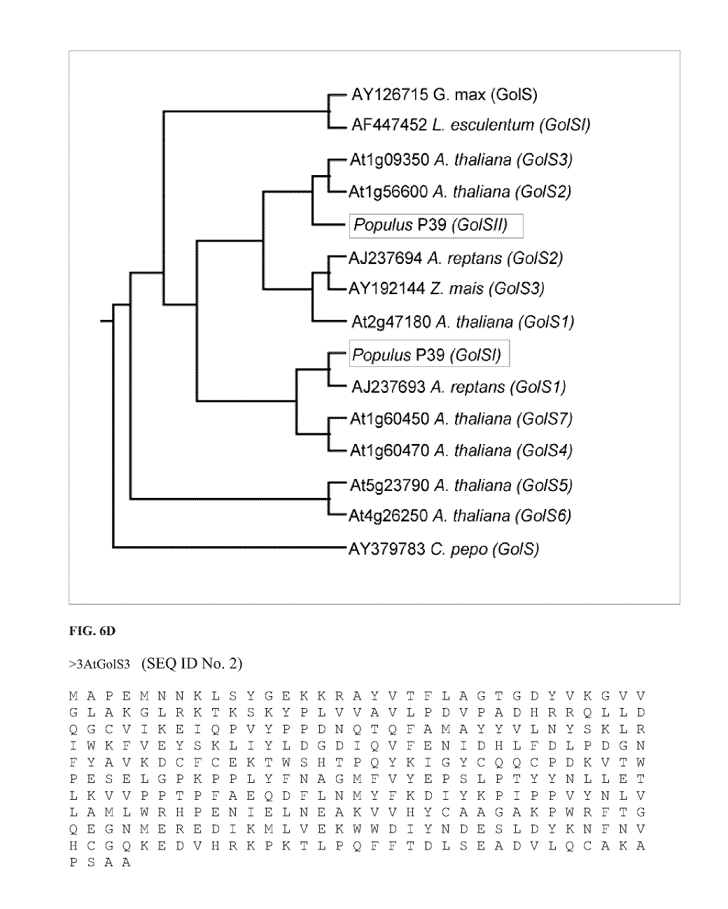

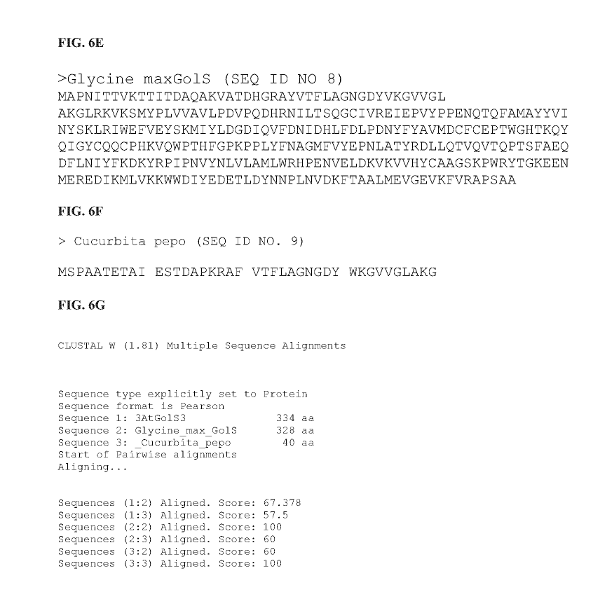

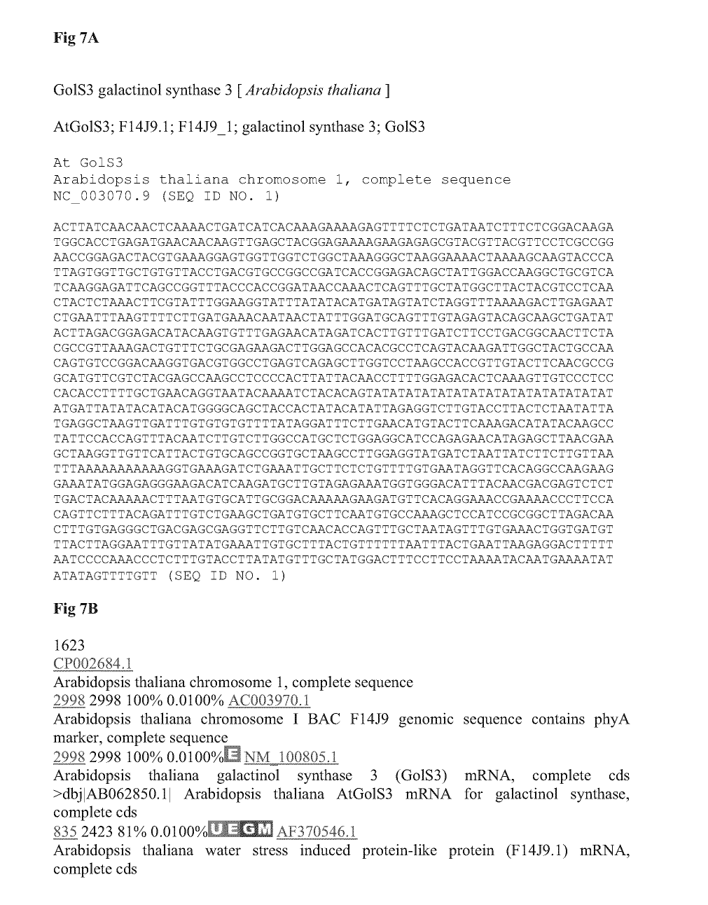

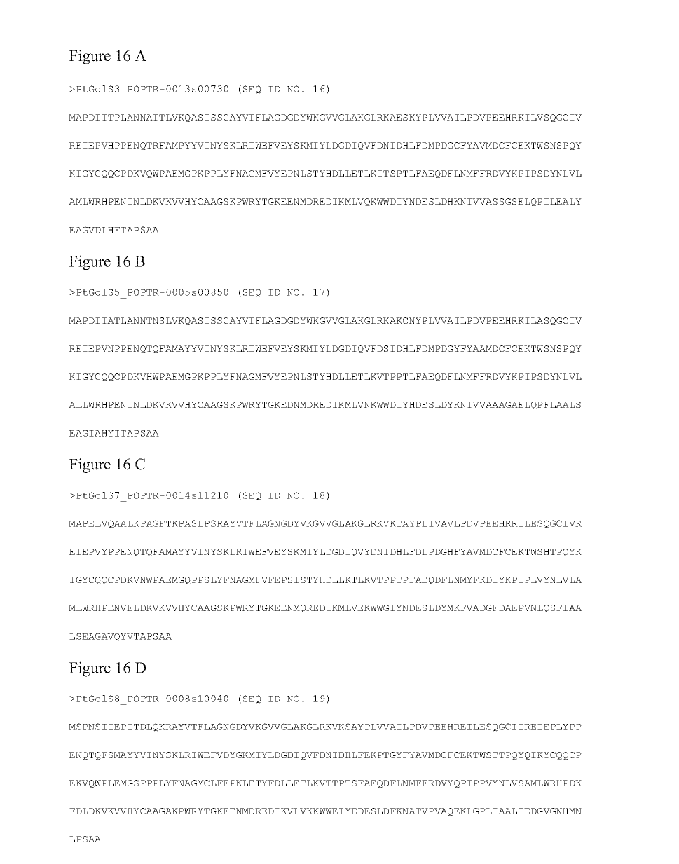

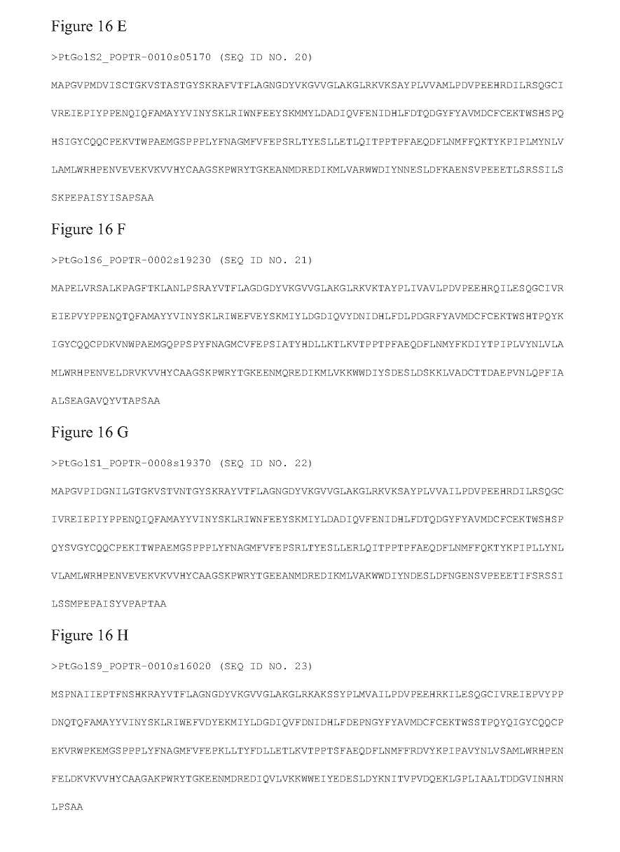

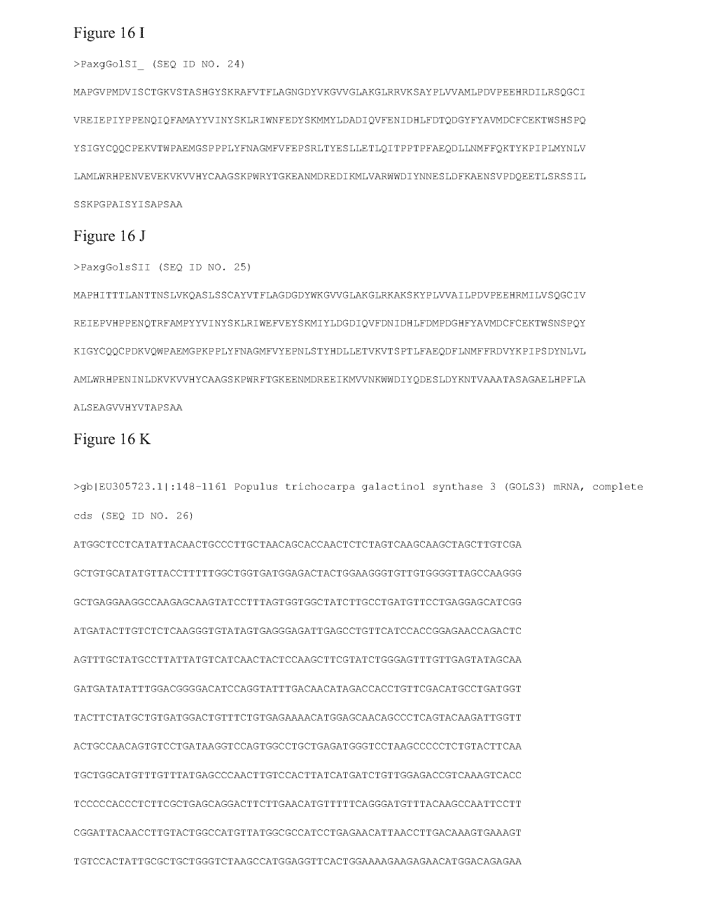

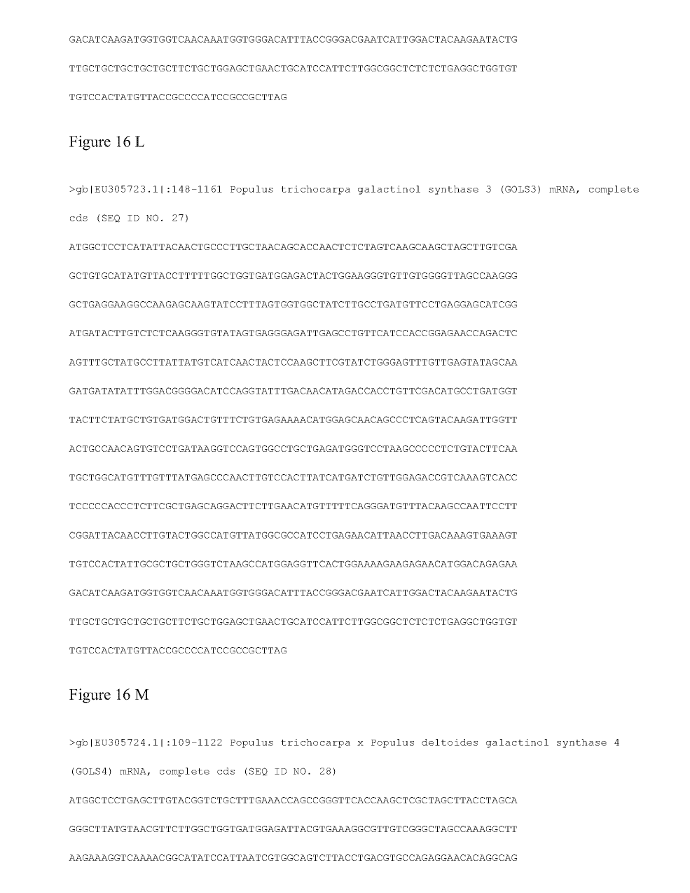

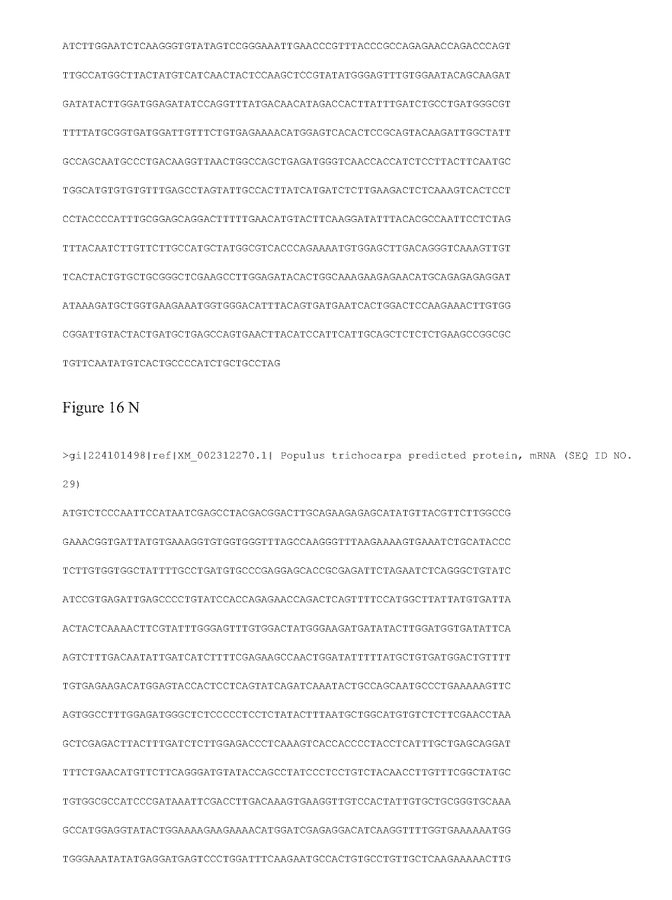

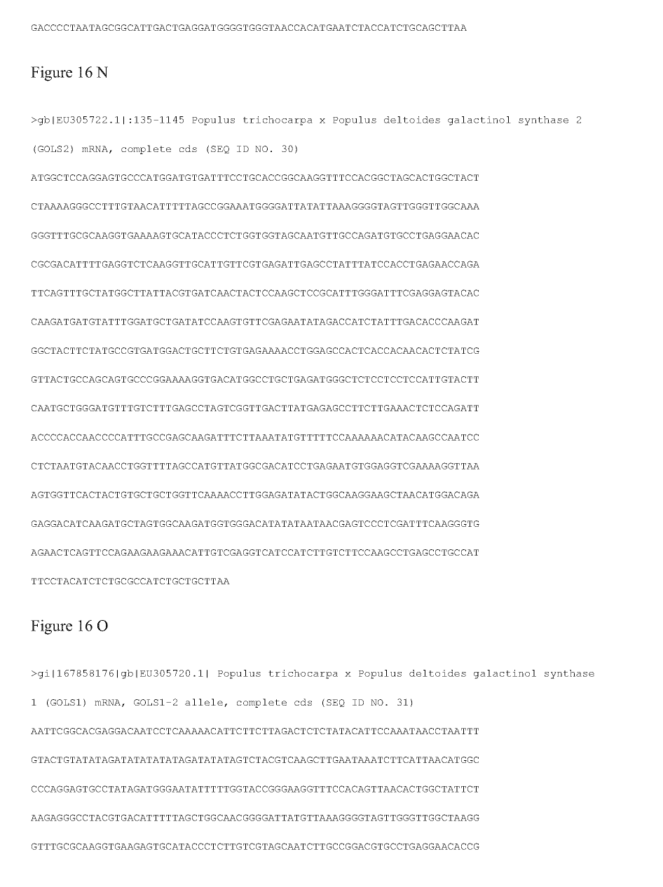

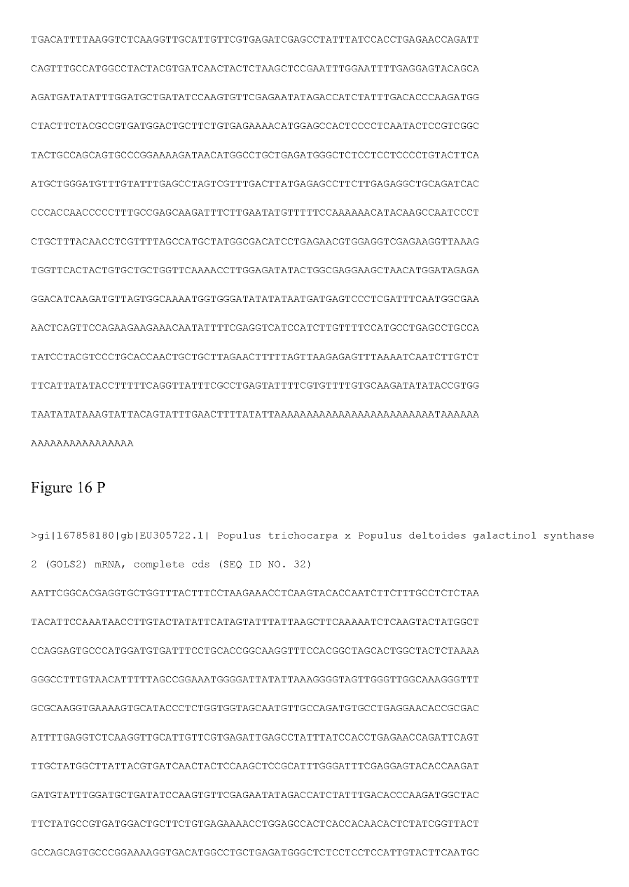

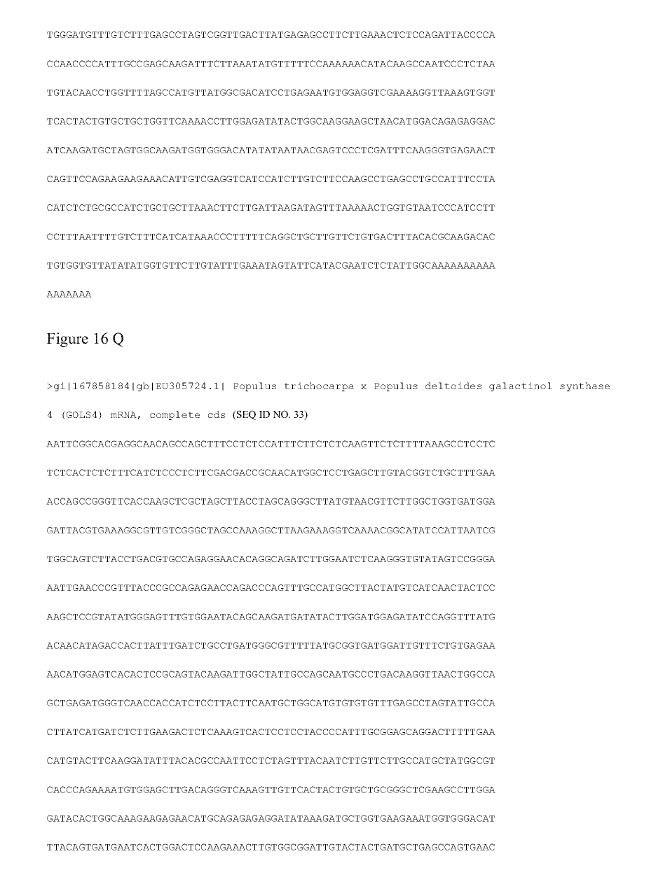

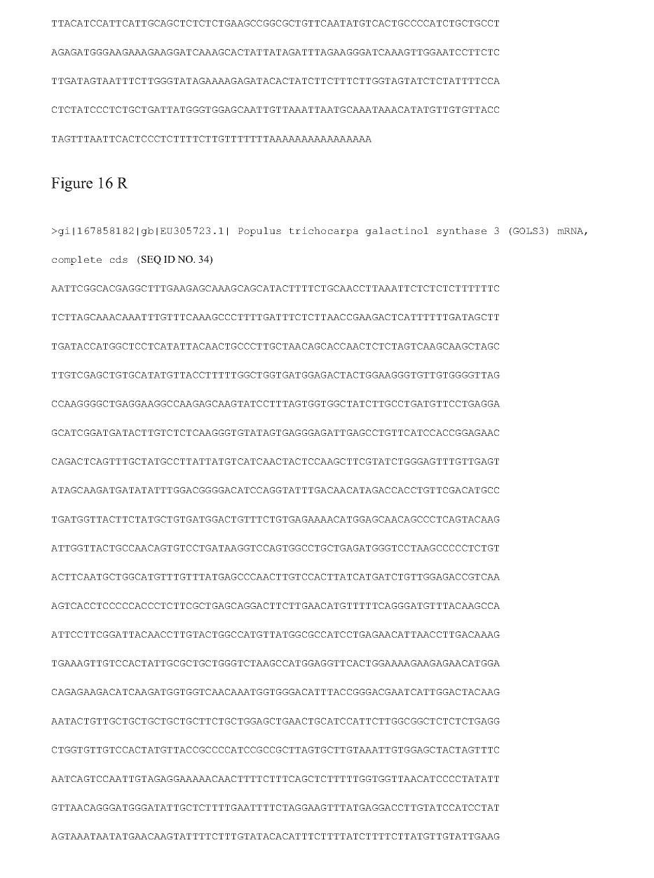

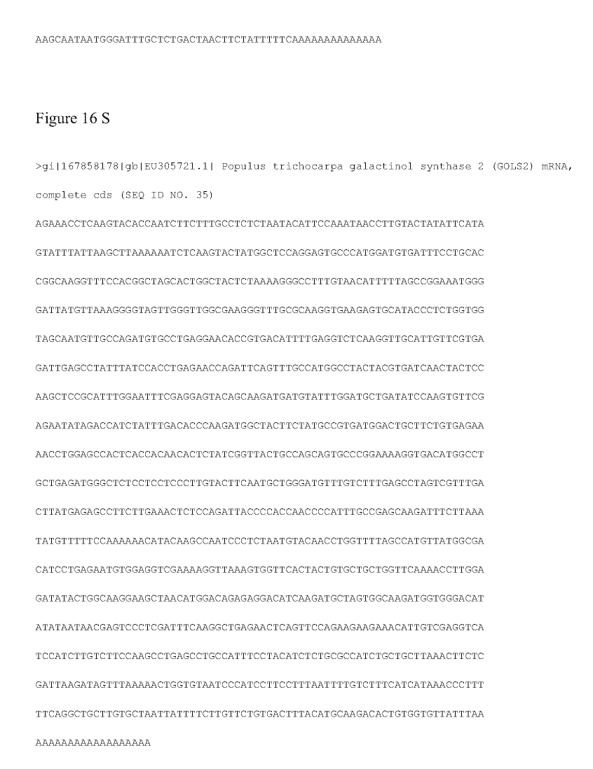

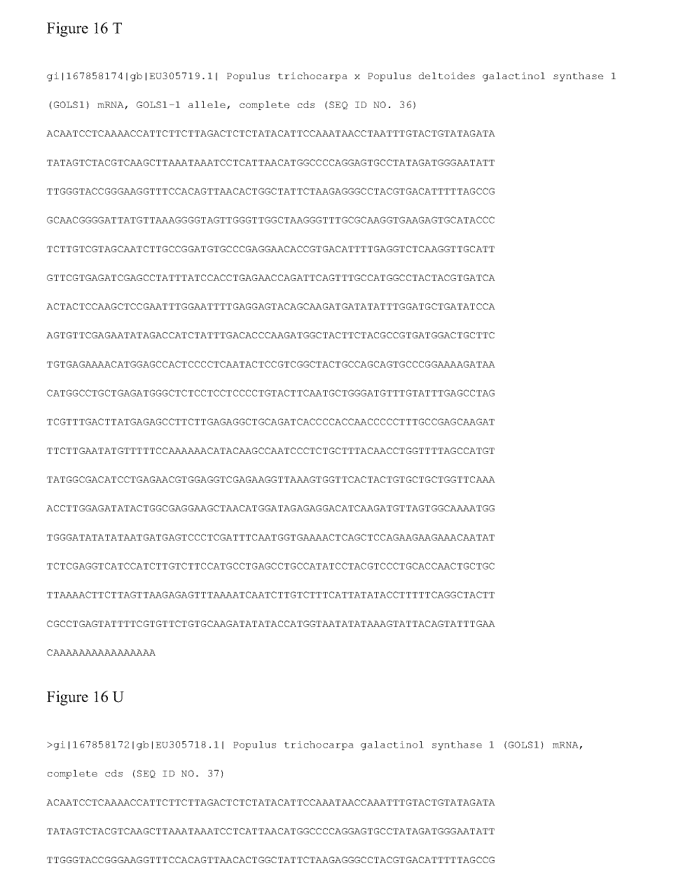

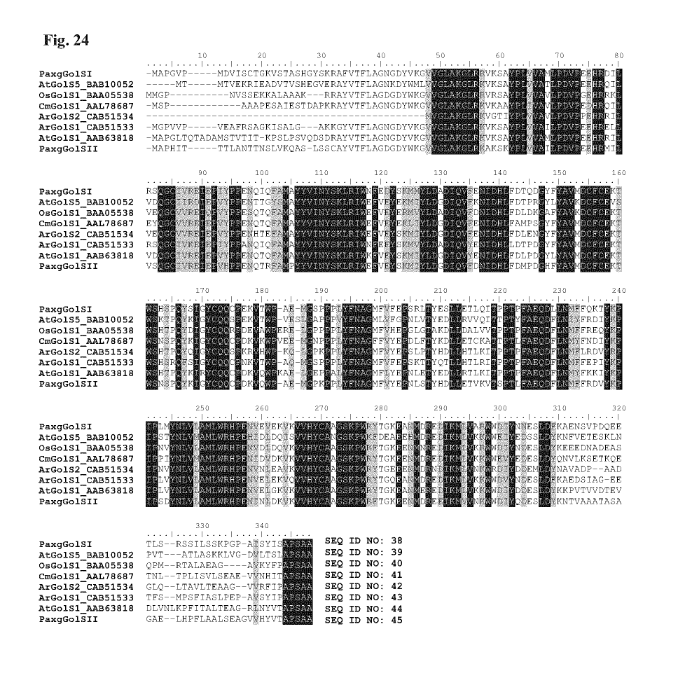

TABLE 2
List of Sequence Identification numbers.

| | | Table/ |
|---|---|---|
| SEQ ID NO: | Description | Figure |
| 1 | cDNA sequence Atg09350 | FIG. 1 |
| 2 | protein sequence At1g09350 | FIG. 2 |
| 3 | Genomic sequence At1g09350 | FIG. 3 |
| 4 | A. thaliana GolS2 protein sequence | FIG. 4A |
| 5 | A. thaliana GolS2 mRNA sequence | FIG. 4B |
| 6 | A. thaliana GolS1 protein sequence | FIG. 5A |
| 7 | A. thaliana GolS1 mRNA sequence | FIG. 5B |
| 8 | Glycine max GolS | FIG. 6E |
| 9 | GolS Cucurbita pepo | FIG. 6B |
| 10 | Query of alignment | FIG. 7 |
| 11 | Subject of alignment | FIG. 7 |
| 12 | Glycine_max_AY126715 (1406 aa) | FIG. 6A |
| 13 | Cucurbita_pepo_AY379783 (1807 aa) | FIG. 6A |
| 14 | Primer AtGolS3.Fw 5′- | page 29 |
| 15 | Primer AtGolS3.Rv 5′- | page 29 |
| 16-25 | protein sequence of Poplar GolS nucleotide | FIG. 16A- |
| | sequence of Poplar GolS | 16J |
| 26-37 | nucleotide sequences of Poplar GolS | FIG. 16K- |
| | | 16U |
| 38 | Amino acid sequence of PaxgGolSI | FIG. 24 |
| 39 | Amino acid sequence of AtGolS5 | FIG. 24 |
| 40 | Amino acid sequence of OSGolS1 | FIG. 24 |
| 41 | Amino acid sequence of CmGolS1 | FIG. 24 |
| 42 | Amino acid sequence of ArGolS2 | FIG. 24 |
| 43 | Amino acid sequence of ArGolS1 | FIG. 24 |
| 44 | Amino acid sequence of AtGolS1 | FIG. 24 |
| 45 | Amino acid sequence PaxgGolSII | FIG. 24 |

### EXAMPLES

### Example 1

### Overexpression of the AtGolS3 Gene in Hybrid Poplar

### Plasmid Construction

The A. thaliana GolS3 (At1g09350) which was previously shown to be cold inducible (Taji et al. 2002) was cloned from cDNA from Columbia ecotype using primers:

AtGolS3.Fw
(SEQ ID NO. 14)
5′-CGCGGATCCATGGCACCTGAGATGAACAACAAGTTG-3′
and
AtGolS3.Rv
(SEQ ID NO. 15)
5′-CGCGAGCTCCTGGTGTTGACAAGAACCTCGCTC-3′.

The galactinol synthase transformation vector was constructed by ligating the cloned AtGolS3 gene using the BamHI and SacI restriction enzymes. Once the vector was confirmed by sequencing, it was transformed into Agrobacterium tumefasciens C58 strain. Suitable transformation vectors are for example pbin, pBinPlus, pBI, pMDC or pRT (Lee and Gelvin in Plant Physiology, Plant Physiology 146:325-332, 2008).

### Agrobacterium Transformation

The transformation was done using the ‘freeze-thaw’ method for direct Agrobacterium transformation (Cellfor Inc., Vancouver, BC). Colonies that grew on the selection medium (i.e., 50 mg l⁻¹ rifamycin and 50 mg l⁻¹ kanamycin) were confirmed as transformants by PCR. Bacterial stock cultures of Agrobacterium strains C-58, carrying the novel construct pSM-3 AtGolS3, vector was grown individually overnight at 28° C. on a gyratory shaker (200 rpm) in LB media with rifamycin (50 mg l⁻¹) and kanamycin (50 mg l⁻¹). Prior to co-cultivation, 1 ml of each bacterial culture was subcultured in MSO medium+100 μM acetosyringone and grown at 28° C. on a gyratory shaker (200 rpm). Bacteria concentrations were determined using a Lambda 45 UV/Vis spectrometer (Perkin Elmer Inc., Wellesley, Mass.). Hybrid poplar P39 was then transformed using the pSM-3-AtGolS3.

### Hybrid Poplar Transformations

Populus alba×grandidentata (P39) leaf discs were harvested from four week-old tissue culture-grown plants using a cork borer. Twenty plates containing 25 leaf discs (7 mm²) per genotype were co-cultivated with 30 ml of bacterial culture in 50 ml Falcon tubes for 30 min at 28° C. in a gyratory shaker (100 rpm). Following co-cultivation, the explants were blotted dry on sterile filter paper and placed abaxially on WPM 0.1 NAA, 0.1 BA and 0.1 TDZ culture medium. The plates were cultured in the dark for two days at room temperature. On the third day, residual Agrobacterium was killed by transferring the leaf discs to WPM media containing 250 mg l¹ cefotoxine and 500 mg l⁻¹ carbenicillin. All plates were kept in the dark for additional two days. Following this period, explants were transferred to selection media WPM with 250 mg l⁻¹ cefotoxine and 500 mg l⁻¹ carbenicillin and 25 mg l¹ hygromycin. Only one shoot per leaf disc was excised and placed on WPM selection media. After 6 weeks, explants were transferred to fresh medium with the same composition. After development, plants were confirmed as being transgenic by genomic DNA screening. They were then subcultured and multiplied on antibiotic free WPM media.

### Plant Growth

Transgenic plants were multiplied in WPM media until approximately ten plants of each line have the same size. The plants were then moved to 2 gallon pots containing perennial soil (50% peat, 25% fine bark and 25% pumice; PH 6.0), and they were maintained on flood tables with supplemental lighting (16 h days) and water daily with fertilized water in the UBC greenhouse, Vancouver, BC.

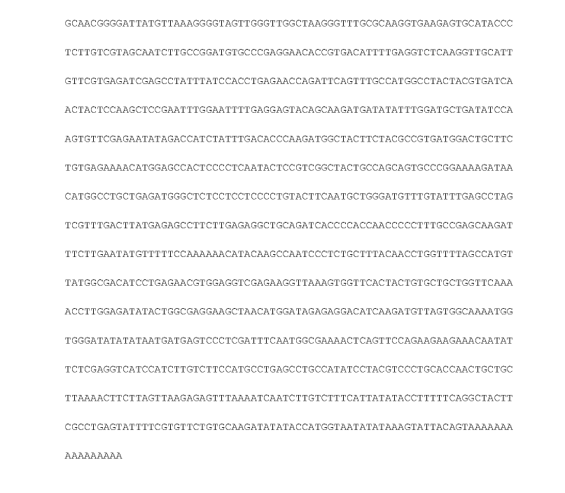

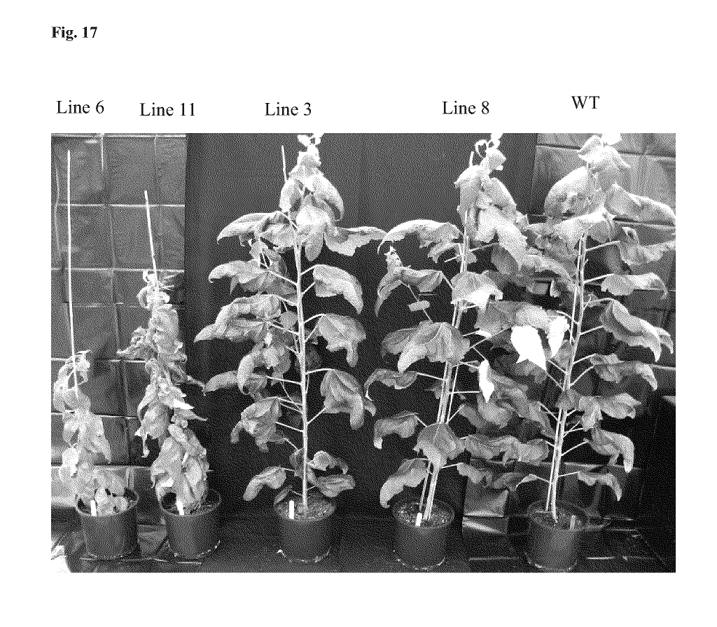

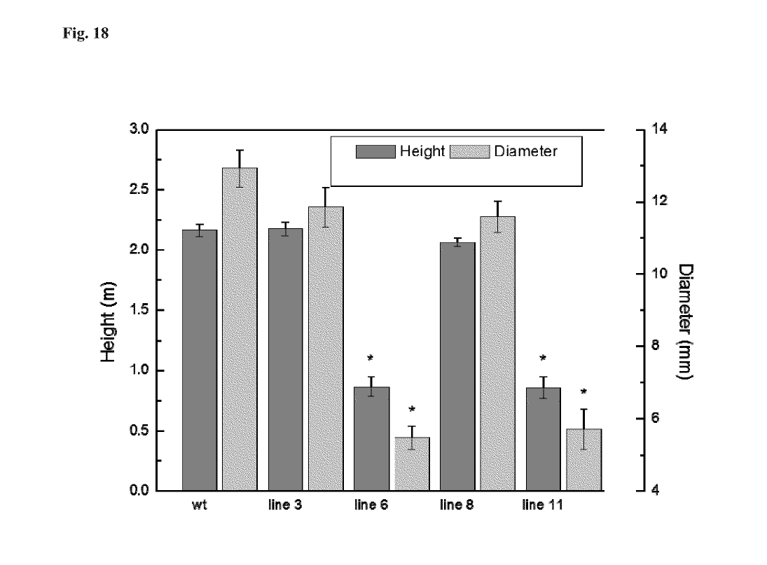

FIG. 17 show five months old greenhouse-grown transgenic poplar trees expressing the Arabidopsis thaliana galactinol synthase 3 gene (AtGolS3) and wild-type. FIG. 18 shows a graph with the height from the base of the stem to the apex, and diameter at 20 cm from the base of the stem of three-month old greenhouse-grown hybrid poplar. Lines 6 and 11 show stunted growth when compared to the wild-type and lines 3 and 8.

### RNA Extraction and Real Time PCR

Total RNA was extracted from approximately 500 mg of frozen ground plant tissue using TRIzol reagent (Sigma) according to the manufacturer's instructions. RNA yield was measured by absorption at 260 nm, and 10 μg was treated with DNAase (Ambion TURBO DNA-free). One μg of the resulting DNA-free RNA was evaluated on a 1% Tris-acetate EDTA agarose gel in order to determine quality. Equal quantities of RNA (1 μg) were used for the synthesis of cDNA with SuperScript II reverse transcriptase (Invitrogen) and (dT)16 primers, according to the manufacturer's instructions. Samples were run in triplicate with Platinum SYBR Green qPCR Master mix (Invitrogen) on an Mx3000p real-time PCR system (Stratagene, La Jolla, Calif., USA). Transcript abundances were determined based on changes in critical threshold (Ct) values relative to elongation initiation factor5A (Gutierrez et al., 2008; Klein et al., 1993).

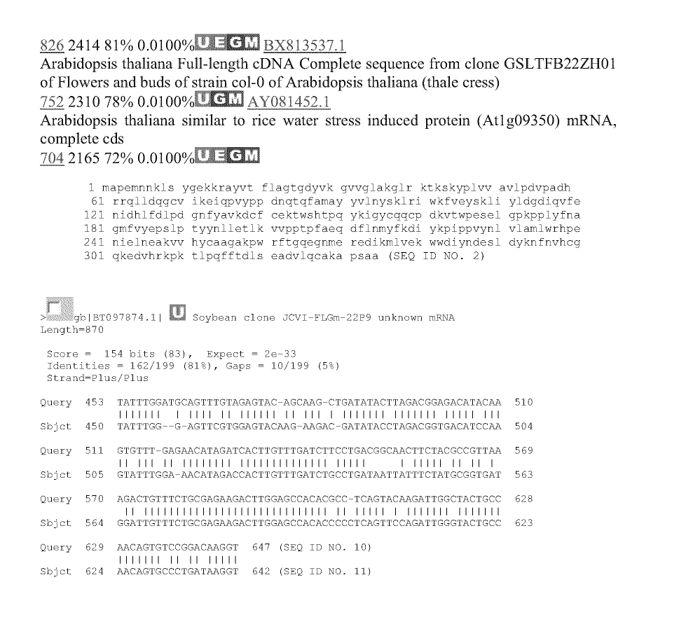

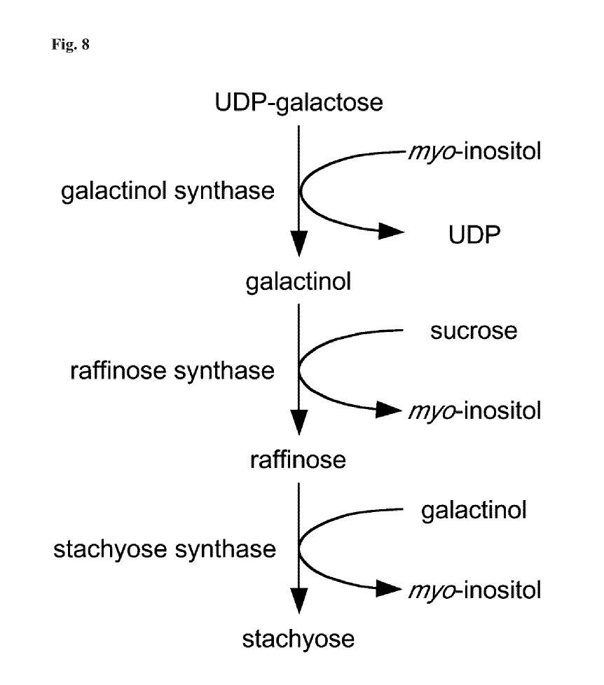

FIG. 9 shows a graph with relative expression 2̂ (−ΔCt) of the At GolS3 in phloem of hybrid poplar. FIGS. 10A and 10B shows a graph with relative expression 2̂ (−ΔCt) of the At GolS3 in 4 tissues in hybrid poplar. Transcript amount of the Arabidopsis thaliana galactinol synthase 3 gene (AtGolS3) in tissues of five months old greenhouse-grown hybrid poplar presented as expression relative to the transcription initiation factor 5A (TIF5A=reference gene) using the formula 2̂ (−ΔCt). Lines 6 and 11 show higher expression of the galactinol synthase 3 gene in the phloem and cambium compared to lines 3 and 8.

### Structural Carbohydrate Analyses

Poplar stem tissue was ground in a Wiley mill to pass a 0.4-mm screen (40 mesh) and Soxhlet extracted overnight in hot acetone to remove extractives. Lignin and carbohydrate content was determined with a modified Klason (Coleman et al., 2009), in which extracted ground stem tissue (50 mg) was treated with 3 mL of 72% H₂SO₄ and stirred every 10 min for 2 h. Samples were then diluted with 112 mL deionized water and autoclaved for 1 h at 121° C. The acid-insoluble lignin fraction was determined gravimetrically by filtration through a pre-weighed medium coarseness sintered-glass crucible, while the acid-soluble lignin component was determined spectrophotometrically by absorbance at 205 nm. Carbohydrate contents were determined by using anion exchange high-performance liquid chromatography (Dx-600; Dionex, Sunnyvale, Calif., USA) equipped with an ion exchange PA1 (Dionex) column, a pulsed amperometric detector with a gold electrode, and a SpectraAS3500 auto injector (Spectra-Physics). Table 8a and 8b show analysis of the structural cell wall carbohydrate and total lignin content of five-month old wild-type and AtGolS3 transgenic poplar trees. Lines 6 and 8 show an increase of up to 178% increase in arabinose concentration when compared to the wild-type. All transgenic lines showed an increased of galactose and glucose (up to 115% and 16%, respectively). Xylose and Lignin content were reduced in all transgenic cell lines (down by 21% and 28%, respectively).

### Soluble Carbohydrate

Soluble carbohydrates (glucose, fructose and sucrose) were extracted from ground freeze-dried tissue overnight at −20° C. using methanol:chloroform:water (12:5:3). Soluble carbohydrates were then analyzed using anion exchange HPLC (Dionex, Sunnyvale, Calif.) on a DX-600 equipped with a Carbopac PA1 column and an electrochemical detector.

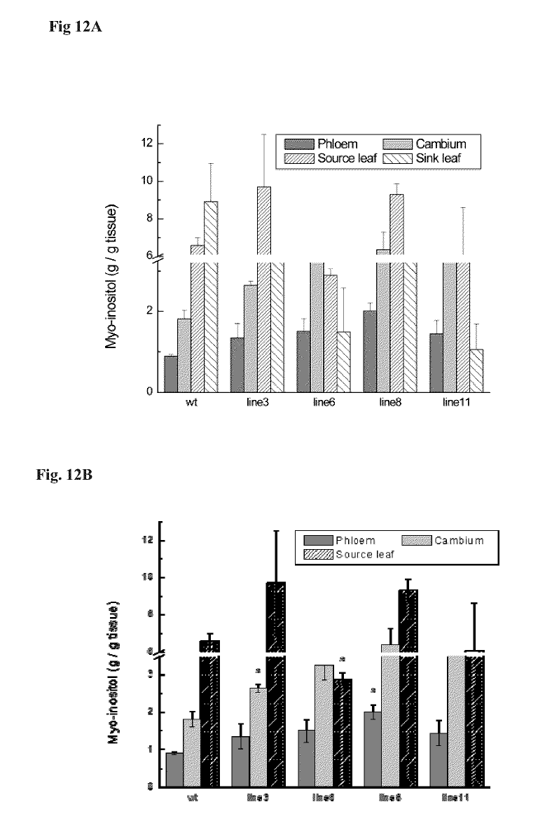

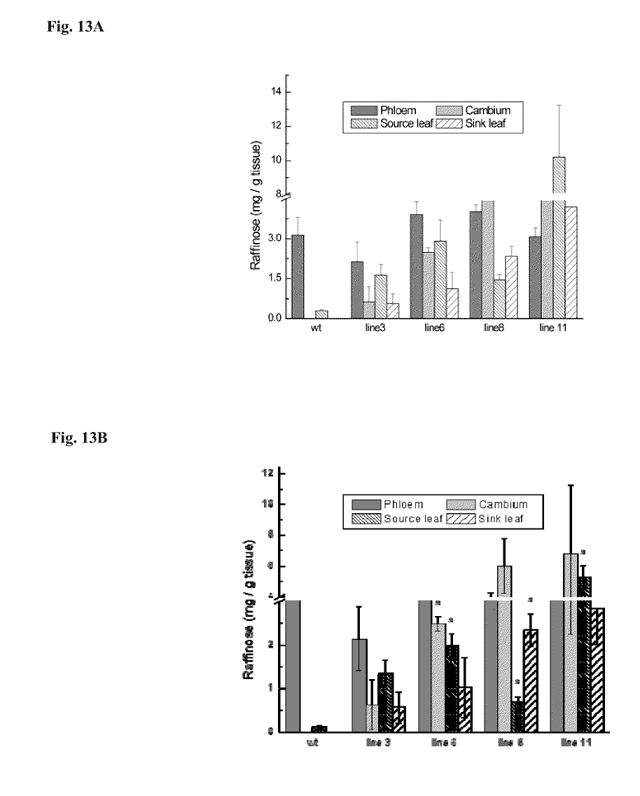

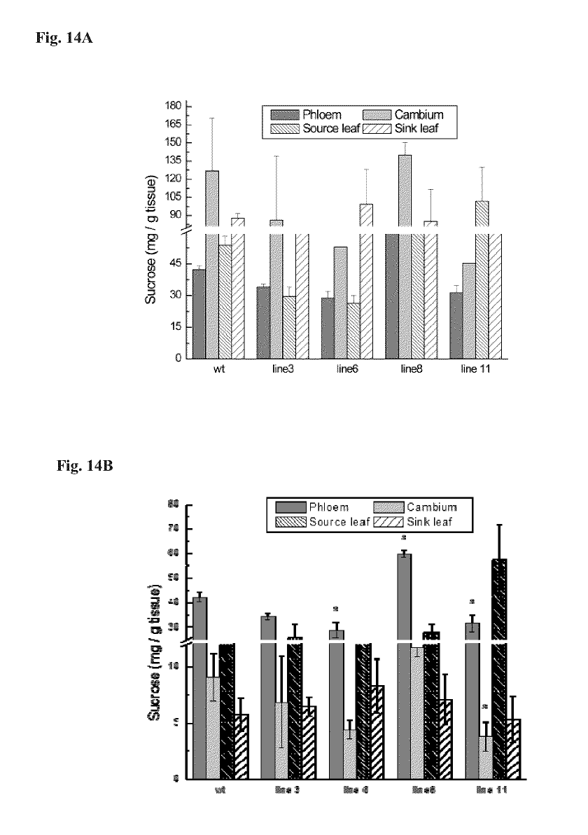

FIGS. 11B and 11A show that the galactinol concentration in the three tested tissues (phloem, cambium and source leaf) are increased in the four transgenic hybrid poplar (lines 3, 6, 8 and 11) when compared to the wild-type.

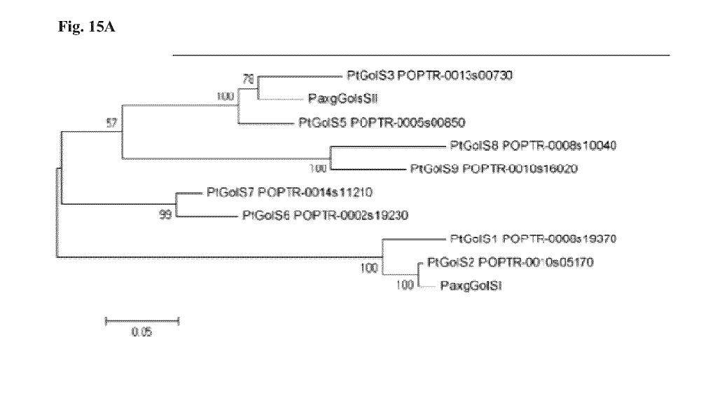

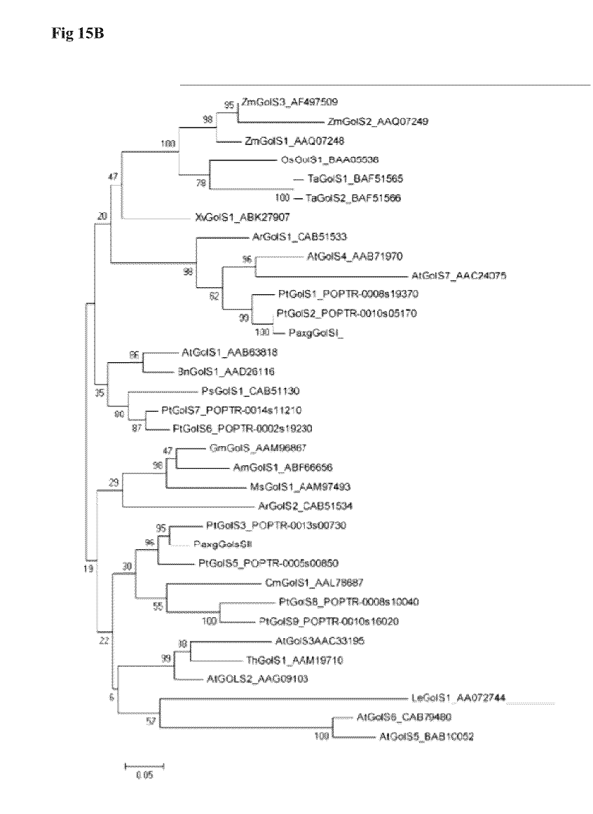

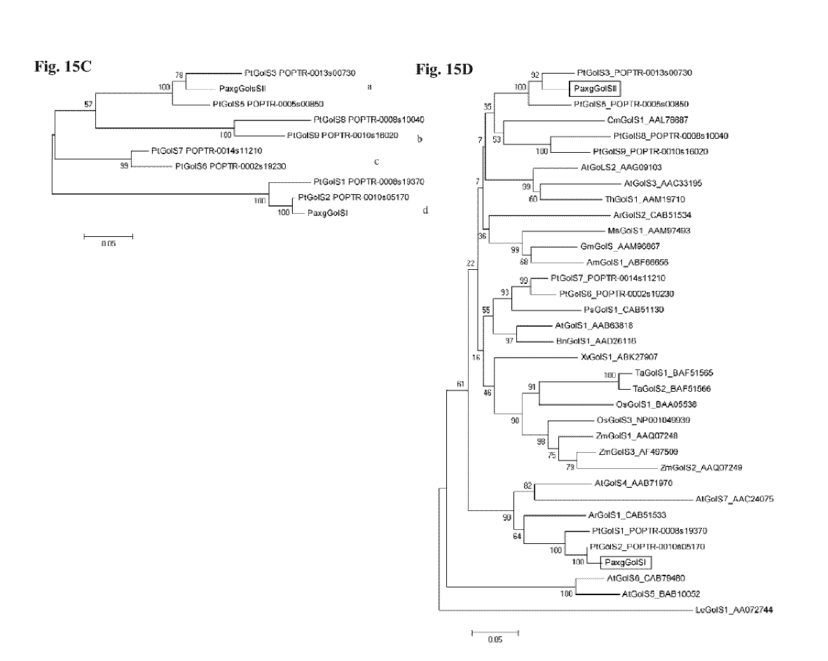

FIGS. 12a and 12b show that the myo-inositol concentration in the phloem and cambium is increased in the four transgenic hybrid poplar (lines 3, 6, 8 and 11) when compared to the wild-type. Furthermore the myo-inositol concentration in the source leaf of transgenic hybrid poplar lines 3 and 8 is higher when compared to the wild-type. Transgenic lines 6 and 8 show in the source leaf a decrease in myo-inositol concentration when compared to the wild-type.

FIGS. 13A and 13B show that the raffinose concentration was increased in the cambiums, source leaf and sink leaf in all four tested lines when compared to the wild-type. Raffinose concentration was also increased in lines 6, 8 and 11 in the phloem when compared to the wild-type.

FIGS. 14A and 14B show that sucrose concentration in the phloem and cambium is less in transgenic hybrid poplar lines 3, 6 and 11 when compared to the wild-type and equal or more in the source leaf for lines 3, 6 and 11.

### Crystallinity and Microfibril Angle

Microfibril angle and cell wall crystallinity were determined by X-ray diffraction using a Bruker D8 Discover X-ray diffraction unit equipped with an area array detector (GADDS) on the radial face of the wood section precision cut (1.69 mm) from the growing stem isolated 5 cm above the root collar. Wide-angle diffraction was used in transmission mode, and the measurements were performed with CuKα1 radiation (λ=1.54 Å), the X-ray source fit with a 0.5 mm collimator and the scattered photon collected by a GADDS detector. Both the X-ray source and the detector were set to theta=0° for microfibril angle determination, while the 2 theta (source) was set to 20° for wood crystallinity determination. The average T-value of the two 002 diffraction arc peaks was used for microfibril angle calculations, as per the method of Megraw et al. (48), while crystallinity was determined by mathematically fitting the data using the method of Vonk (49). Crystallinity measures were pre-calibrated by capturing diffractograms of pure A. xylinum bacterial cellulose (known to be 87% crystalline). Two radii were taken from samples isolated 5 cm above ground on each tree, and these values were averaged for each tree.

Table 7a and 7b show that the microfibril angle in transgenic hybrid poplar lines 11 and 6 is decreased and about equal in lines 3 and 8 when compared to the wild-type. The crystallinity in transgenic hybrid poplar lines 3 and 8 is about equal when compared to the wild-type, while transgenic hybrid poplar lines 11 and 6 show an increase of crystallinity when compared to the wild-type.

### Wood Density

Wood density was measured on the same precision cut samples employed for crystallinity and MFA determination by X-ray densitometry (Quintek Measurement Systems, TN). Pith to bark sections of each tree were scanned at a resolution of 0.0254 mm, and the data are reported as relative density on an oven-dry weight basis, using both radii as the average density per sample.

Table 7a and 7b show that the wood density in all four transgenic hybrid poplar lines (lines 3, 8, 11 and 6) is increased when compared to the wild-type.

### Cross Sectional Staining and Microscopy

Wood samples from six months old poplar trees were soaked overnight in dH₂O. Samples were then cut into 40 μm cross sections with a Spencer AO860 hand sliding microtome (Spencer Lens Co., Buffalo, N.Y. USA) and store in microfuge tubes with dH2O until visualized. Sections were treated with 0.01% Calcofluor white in 1×PBS for 3 min, and then washed 3×5 min in 1×PBS (Falconer and Seagull, 1985). Sections were also treated with 10% phloroglucinol and concentrated HCl. The sections were mounted onto glass slides and visualized with a Leica DRM microscope (Leica Microsystems, Wetzlar, Germany). The pictures were taken with a QICam camera (Q-imaging, Surrey, Canada) and analyzed with OpenLab 4.0Z software (Perkinelmer Inc., Waltham, USA).

Vessel number, length and area were calculated from the 40 μm cross sections stained with phloroglucinol. Three trees per line were analyzed. Four pictures were taken in different zones of the sections and approximately 180 vessel areas were measured per tree. The sections were analyzed on the Carl Zeiss Jena “Jenamed” 2 fluorescence microscope (Carl Zeiss Microscopy LLC, NY, USA). Photos were taken with an Infinity 3 camera (Lumenera Corporation, Ottawa, Canada) and analyzed with the associated Infinity capture program.

FIG. 19 shows shows auto-florescence (A-C) and calcofluor (D-F) staining of wild-type (A, D), AtGolS3 transgenic line 6 (B, E) and transgenic line 11 (C, F) hybrid poplar. Transgenic lines show increased cellulose staining with calcofluor. (Scale bars: 70 μm).

### Antibody Labelling

The cross section samples preparations described above were also subject to antibody labeling. Briefly, non-specific protein binding was blocked with 5% BSA in TBST (10 mM Tris-buffer, 0.25 M NaCl, pH 7, with 0.1% Tween) for 20 min. Sections were then treated with diluted primary antibody (1:50) anti-β-(1-4)-D-mannan (catalogue #400-4) monoclonal antibody (Biosupplies Australia Pty Ltd, Melbourne, Australia), anti-xylan LM10 antibody (kind gift of Dr. J. Paul Knox, (www.plantprobes.co.uk) or CCRC-M7 against RGI (Puhlmann et al., 1994) at room temperature for 1 hour. The sections were then washed twice with TBST for 5 minutes. The diluted secondary antibody (Alexa 543: antirat or antimouse) 1:50 was then added, incubated for 1 hour and washed twice with TBST. Samples were mounted on glass slides with 90% glycerol or anti-fade mounting media. Fluorescent localization was observed on Leica DRM (Leica Microsystems, Wetzlar, Germany) light microscope using a Texas Red filter, and images captured with a QICam camera (Qimaging, Surrey, Canada) and analyzed with OpenLab 4.0Z software (Perkinelmer Inc., Waltham, USA). The antibody tagged sections were stored at 4° C. in TBST 1× in microfuge tubes if they were not used immediately.

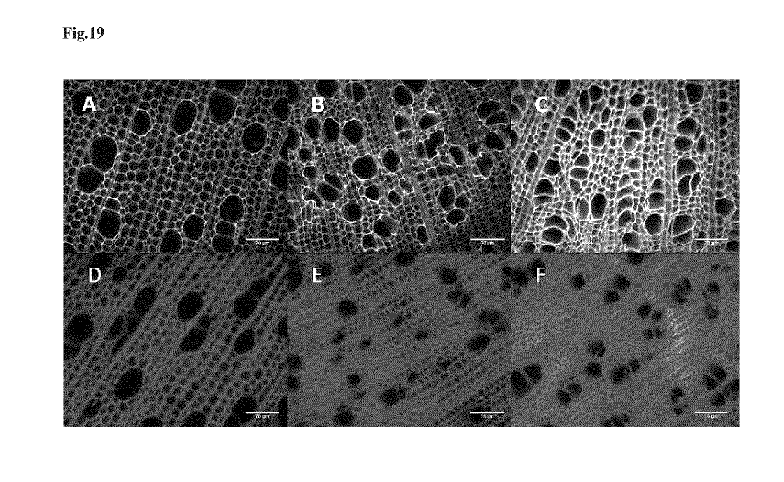

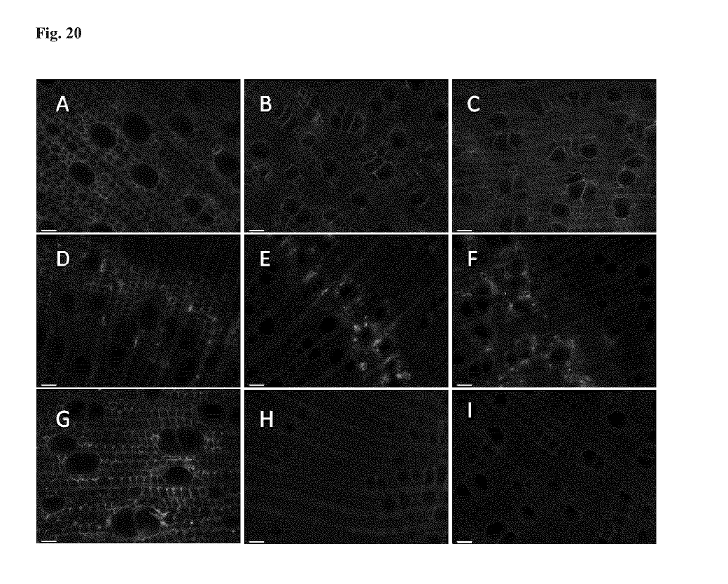

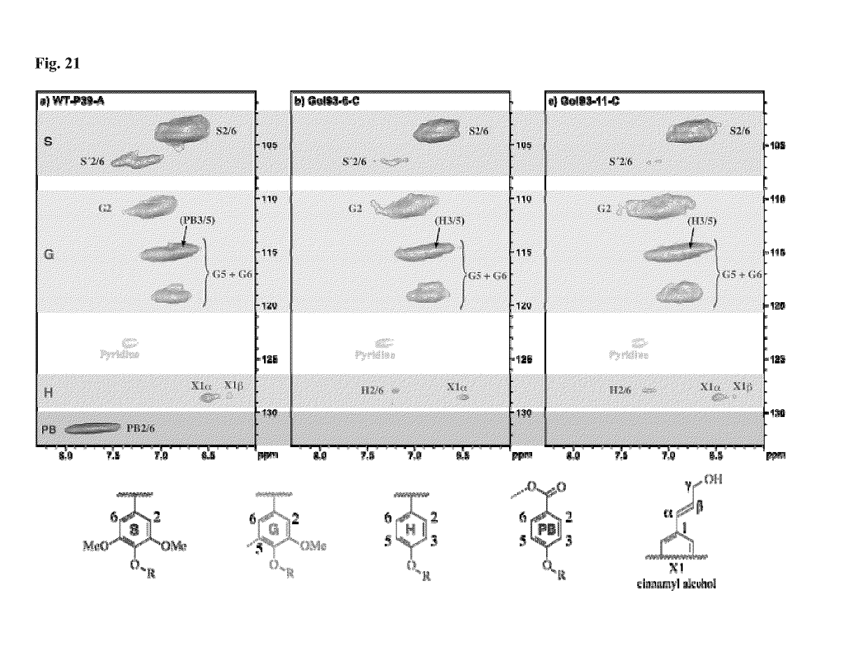

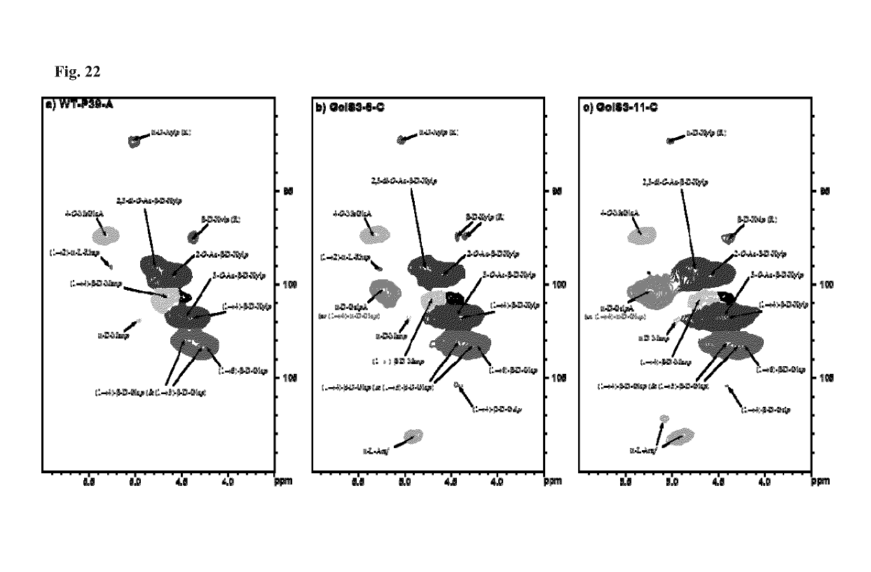

FIG. 20 shows immunofluorescence labeling of xylem tissue from wild-type (A, D and G); AtGolS3 transgenic line 6 (B, E and H) and AtGolS3 transgenic line 11 (C, F and I) hybrid poplar. Tissue was label with the anti-xylan LM10 antibody (A-C); the anti-RGI CCRCM7 antibody (D-F) and the anti-mannan antibody (G-I).

### Cell Wall Characterization

Fibre length was determined on a 1 cm segment isolated 10 cm above the root collar. Samples were macerated in Franklin solution (1:1, 30% peroxide:glacial acetic acid) for 48 h at 70° C. Following the reaction, the residual solution was decanted and the tissue washed extensively with DI water under vacuum until a neutral pH was achieved. The fibrous sample was then resuspended in 10 ml of DI water and diluted to attain a fibre count of 25-40 fibres per second on the Fibre Quality Analyzer (FQA; OpTest Equipment Inc. Hawkesbury, Ont. Canada). Fibre length for each samples was assessed on 10,000 fibres.

FIG. 23 shows that the fiber length in transgenic hybrid poplar lines 3 and 8 was about equal to the fiber length of the wild-type. Transgenic hybrid poplar lines 6 and 11 had decreased fiber length when compared to the wild-type. Similarly, the fiber width of transgenic hybrid poplar lines 3 and 8 was about equal when compared to the wild-type, whereas the fiber width of transgenic hybrid poplar lines 6 and 11 was less when compared to the wild-type.

### Whole Plant Cell Wall Characterization Using Solution-State 2D NMR

Whole plant cell wall were characterized using solution-state 2D NMR as described in Mansfield et al. Nature Protocols, 2012, Vol. 7, No. 7 (which is hereby incorporated by reference). Briefly, the protocol comprises (i) cell wall isolation, (ii) fine grinding (by ball milling), (iii) swelling or dissolution (and possibly acetylation) in a simple mixed solvent system and (iv) acquisition and interpretation of 2D NMR spectra on the entire cell wall material.

### Procedure

The protocol comprises procedures for (i) the preparation and extraction of a biological plant tissue, (ii) solubilization strategies for plant material of varying composition and (iii) 2D NMR acquisition (for typically 15 min-5 h) and integration methods used to elucidate lignin subunit composition and lignin interunit linkage distribution, as well as cell wall polysaccharide profiling.

### Sample Setup•Timing ˜5 d

1| Allow plant biomass to air-dry until a constant moisture content is attained. (approximately 2-3 d at ambient temperature),
2| Grind the dried plant biomass in a Wiley mill fitted with a 40-μm mesh screen and obtain the flour that passes through the mesh,
3| Extract the ground material by either (option A) Soxhlet extraction or (option B) solvent extraction. Samples with high protein content, such as immature tissues or photosynthetic material (e.g., Arabidopsis, immature grasses), should be subjected to a more extensive solvent extraction 71,72 (option C). Extraction is essential to isolate the cell wall and remove the nonstructural components (i.e., extractives) that may appear as ‘pseudo-lignin’ in the samples and distort the estimation of cell wall components. Unless there is an interest in characterizing the extractives' composition (by gas chromatography-mass spectrometry), the material is simply discarded.

### (A) Soxhlet Extraction

(i) Extract the ground material overnight (minimum of 8 h) with 95:5 acetone/water on a Soxhlet apparatus (˜70° C.). Note that boiling chips may be used to control solvent boiling.

### (B) Solvent Extraction

(i) Add 200-4,500 mg of plant material to a 50-ml conical centrifuge tube.
(ii) Add 40 ml of water and sonicate for 20 min,
(iii) Centrifuge the samples for 10 min at 3,480 r.p.m, (˜2,800 g, 21° C.).
(iv) Remove the solvent by decanting or aspirating and discard it.
(v) Repeat the water addition, sonication, centrifugation and solvent removal two additional times.
(vi) Add 40 ml of 80% (vol/vol) ethanol and sonicate for 20 min,
(vii) Centrifuge the samples for 10 min at 3,480 r.p.m. (˜2,800 g, 21° C.)
(viii) Remove the solvent by decanting or aspirating and discard it.
(ix) Repeat the addition of 80% (vol/vol) ethanol, sonication, centrifugation and solvent removal two additional times.
(x) Add 40 ml of 100% acetone and sonicate for 20 min.
(xi) Centrifuge the samples for 10 min at 3,480 r.p.m. (˜2,800 g, 21° C.).
(xii) Remove the solvent by decanting or aspirating and then discard. Hatfield 71,72 has found that aspirating the liquid off may work better as it is easier to control so as to not disturb the pellet.

### (C) Extensive Solvent Extraction

(i) Add 200-1,500 mg of plant material to a 50-ml conical centrifuge tube.
(ii) Add 30 ml of 50 mM NaCl and mix thoroughly by vortexing.
(iii) Place the solution in a 4° C. refrigerator overnight.
(iv) Centrifuge the samples for 10 min at 3,480 r.p.m, (˜2,800 g, 1° C.), remove the solvent by decanting or aspirating, and then discard it.
(v) Add 40 ml of 80% ethanol and sonicate for 20 min.
(vi) Centrifuge the samples for 10 min at 3,480 r.p.m. (˜2,800 g, 1° C.), remove the solvent by decanting or aspirating, and then discard it.
(vii) Repeat the addition of 80% (vol/vol) ethanol sonication, centrifugation and solvent removal two additional times.
(viii) Add 40 ml of 100% acetone and sonicate for 20 min.
(ix) Centrifuge the samples for 10 min at 3,480 r.p.m. (˜2,800 g, 1° C.); remove the solvent by decanting or aspirating and discard it.
(x) Add 40 ml of CHCl3/methanol (1:1) and sonicate for 20 min,
(xi) Centrifuge the samples for 10 min at 3,480 r.p.m. (˜2,800 g, 1° C.); remove the solvent by decanting or aspirating and discard it.
(xii) Add 40 ml of 100% acetone and sonicate for 20 min.
(xiii) Centrifuge the samples for 10 min at 3,480 r.p.m. (˜2,800 g, 1° C.), and remove the solvent by decanting or aspirating and then discard.

4| Dry the extract-free plant material by either freeze-drying using standard lyophilization techniques or oven-drying at ˜50° C. (heating should not surpass 50° C.) until the sample moisture content has stabilized (generally, 24 h of drying at ˜50° C. is sufficient).

### Starch Removal (Optional•Timing ˜5 h)

Certain plant cell wall samples may inherently have substantial amounts of starch, and it is useful to selectively characterize starch-free plant biomass. The removal of starch from the cell wall matrix will permit the more accurate assignment of the neutral sugar composition. The following steps can be included in the sample preparation to remove starch from isolated cell wall material (isolated by any of the above methods, most rapidly via the alcohol-insoluble residue method described above).

5| Preheat a water bath to 90° C.
6| Add 200-1,500 mg of plant material to a 50-ml conical centrifuge tube (this can be extract-free ground plant material, or plant material that had previously been deproteinated (see above—step B and C).
7| Add 25 ml of 10 mM Tris-acetate buffer (pH 6.0) and mix thoroughly.
8| Cap the centrifuge tubes and place them in a hot water bath. Avoid contact with hot water and steam.
9| Incubate the samples in a submersible rack for 2 h at 90° C.
10| Remove the samples from the water bath and allow them to cool to 55-60° C.
11| While the samples are cooling, in 5 ml of 10 mM Tris-acetate buffer (pH 6.0) add the equivalent of 20 and 40 units of amylase and amyloglucosidase, respectively, per gram of cell wall sample.
12| Add the 5-ml enzyme preparation to the sample (55° C.) and maintain it at 55° C. in the water bath for 2 h. The samples should not be permitted to remain in the enzyme solutions at temperatures below 55° C. This is a precaution against cell wall-hydrolyzing enzymes surviving the 55° C. temperature treatment and becoming active once the temperature begins to decrease.
13| Centrifuge the samples for 10 min at 3,480 (˜2,800 g, 4° C.).
14| Remove the enzyme solution by decanting or aspirating and discard.
15| Add 40 ml deionized water and mix thoroughly.
16| Centrifuge the samples for 10 min at 3,480 r.p.m. (˜2,800 g, 4° C.).
17| Remove water by decanting or aspirating, and then discard; repeat two additional times.
18| Add 40 ml of 100% acetone and sonicate for 20 min.
19| Centrifuge the samples for 10 min at 3,480 r.p.m. (˜2,800 g, 4° C.).
20| Remove the solvent by decanting or aspirating, and then discard; repeat two additional times.

Milling•Timing 1-2.5 h

21| Grind plant material derived from either Step 4 or Step 20 using the ball-milling procedure. This is achieved by using a Retsch PM100 mill fitted with one or two 50 ml ZrO₂ grinding jars and 10×10 mm ball bearings, set at 600 r.p.m., or by using a Fritsch Planetary micro mill Pulverisette 7 premium line with two 20-ml jars and the same conditions. The grinding conditions vary by species and the amount of plant material (extract-free flour) being ground. Examples for conditions are as follows:

TABLE 3

| | | | Total |
|---|---|---|---|
| Plant | Amount (mg) | Milling protocol | time (min) |
| Poplar | 200 | 5 × 10 min with 5 min | 70 |
| | | pauses in between | |
| Pine | 200 | 5 × 20 min with 10 | 140 |
| | | min pauses in between | |
| Arabidopsis and | 200 | 5 × 5 min with 5 min | 45 |
| grasses | | pauses in between | |

The Fritsch mill must use balanced samples. Short grinding periods and a break in between (repeated several times) limits sample heating. Very fine milling using a ball mill is required for cell wall dissolution (or gel formation) for NMR.

### Preparation for NMR

22| Two types of NMR samples can be prepared using the ground plant material. Use option A to prepare acetylated cell walls and option B to prepare native cell walls. When preparing acetylated cell walls, if higher-resolution work is planned, or if longer relaxation is required (e.g., for long-range 13C-1H (HMBC) experiments), removing trace metals (usually of plant origin) using EDTA is beneficial (Step 22A(ix-xii)).

### (A) Dissolution and Acetylation•Timing ˜2 d

(i) Suspend 100 mg of extract-free, ball-milled cell wall sample in 2 ml of DMSO.
(ii) Add 1 ml of NMI with constant stirring, using a magnetic stirrer. Note: A clear solution will form in 3 h or less (depending on the nature of the sample).
(iii) Once dissolution is achieved, add 0.5 ml (excess) of acetic anhydride to the solution and stir the mixture for an additional 1.5 h.
(iv) Transfer the resulting clear brown solution to 300 ml of deionized water and allow it to stand overnight.
(v) Centrifuge the solution for 10 min at room temperature 21° C. and 3,480 r.p.m. (˜2,800 g, 1° C.) to recover the precipitate.
(vi) Wash the recovered precipitate with 100 ml of deionized water.
(vii) Dry the sample under vacuum (<100 mTorr, <15 Pa) at room temperature; after drying, weigh the sample.
(viii) Lyophilize the sample, using standard procedures, in order to remove all remaining solvents. Note that the weight of acetylated cell walls is typically 137-140% of the weight of the original cell wall material.
(ix) (Optional) Trace-metal removal: wash freeze-dried acetylated plant material on a Nylon filter with 100 ml (excess) of 6 mM EDTA. Alternatively, for more effective metal removal, dissolve the acetylated cell wall in 100 ml of chloroform and then extract the sample with 10 ml of 6 mM EDTA three times.
(x) Dry the separated chloroform layer over anhydrous sodium sulfate, remove the sodium sulfate by vacuum filtration through a medium-porosity sintered glass funnel and remove the solvent at reduced pressure on a rotary evaporator.
(xi) Dissolve 30-50 mg of acetylated cell wall material in 0.5 ml of CDCl3 or DMSO-d6.
(xii) Transfer acetylated cell wall solution to a NMR (5 mm outer diameter) tube. The sample is stable almost indefinitely, although it is best kept refrigerated and in the dark.
(B) Gelling without Derivatization•TIMING ˜5 h
(i) Transfer 30-60 mg of extract-free, ball-milled plant cell wall material (as a dry powder) directly into a 5-mm NMR tube.
(ii) Distribute the sample as well as possible off the bottom and up the sides of the horizontally positioned NMR tube before introducing the solvent.
(iii) Add 500 μl of premixed DMSO-d6/pyridine-d5 (4:1) directly into the NMR tube for each sample. The NMR solvent mixture is carefully introduced (via a syringe), spreading it from the bottom of the NMR tube, along the sides and toward the top of the sample. Pyridine-d₅ with a purity of 99.5 atom % D is used for most cell wall samples, but pyridine-d₅ of enhanced purity (‘100’; min. 99.94 atom % D) can be used for grass (e.g., corn) samples to minimize interference between the residual solvent (pyridine) peaks and the correlations from ferulate and p-coumarate moieties.
(iv) Sonicate the NMR tubes in an ultrasonic bath for 1-5 h (depending on the sample) until the gel becomes apparently homogeneous. The final sample height should be approximately 4-5 cm in the NMR tube. An alternative method is to run (via a second external magnet) a cylindrical Neodymium magnet (⅛ inch diameter×½ inch long) inside the NMR tube to mix the sample. If the alternative method is used, it is crucial to remove this magnet before placing the tube in the NMR magnet.

### Acquisition of NMR Spectra•Timing 15 Min-5 h

23| Acquire 2D 1H-13C HSQC spectra using a standard Bruker pulse program (‘hsqcetgpsisp.2’ or ‘hsqcetgpsisp2.2’).
Adiabatic-pulse variants of the HSQC experiment seem to give the least artifacts and the most uniform profiles relatively independently of 1JC-H coupling constants. The current optimal conditions are as follows:

TABLE 4

| | |
|---|---|
| Spectrometer | 400-700 MHz, with cryoprobe |
| | (1H coil/preamp cooling only necessary) |
| Bruker Pulse program | hsqcetgpsisp.2 or hsqcetgpsisp2.2 |
| | (Adiabatic-pulse HSQC) |
| Sample spinning | The acquisition is run without sample spinning |
| SW(H) | 12 p.p.m. (from 11 to −1 p.p.m., centered at 5 |
| | p.p.m.) |
| SW(C) | 220 p.p.m. (from 220 to −20 p.p.m., centered at 90 |
| | p.p.m.) |

TABLE 5

| | Fully dissolved | | | |
|---|---|---|---|---|
| | acetylated samples | | Gel samples in | |
| | in CDCl3 or | | DMSO -d₆/ | |
| | DMSO -d₆ | | pyridine-d₅ | |
| AQ (f2, proton, acquisition time) | ≦200 | ms | ~100 | ms |
| AQ (f1, carbon, ‘acquisition time’) | ~8 | ms | ~8 | ms |
| | (TD1*IN0) | | (TD1*IN0) | |
| D1 (relaxation delay) | 1 | s | 0.5 | s |

| | | |
|---|---|---|
| NS (number of scans) | To give total | To give total |
| | acquisition | acquisition |
| | time of 1-3 h | time of 5 h |

TD1 is the number of experiments;
IN0 is the increment delay for successive experiments;
F1 and F2 refer to the two frequency axes.

For both sample types, more scans may be required for improved and more detailed interpretation; on a cryoprobe-equipped instrument, surprisingly detailed and useful spectra are readily acquired in ˜15 min.

### Processing•Timing ˜1 Min

24| Processing can be completed off-line, e.g., via Apple Macintosh, Microsoft Windows or Linux data stations running the Topspin 3.x software, but it can also be completed directly on the instrument. The final 2D data matrix size is typically 2,048×1,024 data points.

TABLE 6

| | |
|---|---|
| F2 (proton frequency co-ordinates) | GM, GB = 0.001, LB approximately |
| | −0.1 to −0.3 (Gaussian apodization to |
| | match FID free induction decay) |
| F1 (carbon frequency co-ordinates) | QSINE 2 (cosine-bell apodization) |

GM = gaussian multiplication
GB and LB are the gaussian broadening factor and the exponential broadening factors respectively.

### Contour Volume Integration•Timing ˜30 Min

25| Measure volume integrals on isolated contours using the processing software (e.g., Bruker Topspin 3.x). For S/G/H measurements, the S2/6, G2 and H2/6 contours are used as the C-H pairs have similar environments; the G2 integral is logically doubled.

### •TIMING

Steps 1-4, sample setup: ˜5 d
Steps 5-20, starch removal (optional, 5 h):
Step 21, milling: 1-2.5 h
Step 22, preparation for NMR: variable; 5 h-2 d
Step 23, acquisition of NMR spectra: 15 min-5 h
Step 24, processing 1 min:
Step 25, contour volume integration (30 min).

TABLE 7a
Average (standard error) wood density, microfibril angle
and cell wall crystallinity of galactinol synthase overexpressing
transgenic poplar compared to wild-type poplar.

| | | | | | Cell Wall | |
|---|---|---|---|---|---|---|
| | Density | St. | Microfibril | St. | Crystal- | St. |
| | (kg/m3) | Error | Angle (°) | Error | linity (%) | Error |
| Wild-type | 293.2 | (9.6) | 10.2 | (0.5) | 36.7 | (1.2) |
| Line 3 | 332.2 | (49.6) | 10.4 | (0.2) | 36.0 | (1.5) |
| Line 8 | 347.1 | (3.8) | 10.2 | (0.5) | 34.7 | (0.9) |
| Line 11 | 549.2 | (110.9) | 7.4 | (0.4) | 45.0 | (1.7) |
| Line 6 | 564.1 | (14.5) | 7.7 | (0.3) | 41.3 | (3.4) |
| Line 10 | 302.6 | (14.9) | | | | |
| Line 2 | 279.0 | (5.3) | | | | |
| Line 5 | 300.3 | (22.8) | | | | |
| Line 9 | 296.9 | (4.7) | | | | |

TABLE 7B
Average (standard error) wood density, microfibril
angle and cell wall crystallinity of galactinol synthase
overexpressing transgenic poplar compared to wild-type poplar.

| | Density | Microfibril Angle | Crystallinity |
|---|---|---|---|
| | (kg/m³) | (°) | (%) |
| Wild Type | 293.2 (9.6) | 10.2 (0.5) | 46.7 (1.2) |
| Line 3 | 332.2 (49.6) | 10.4 (0.2) | 46.0 (1.5) |
| Line 8 | 347.1 (3.8) | 10.2 (0.5) | 44.7 (0.9) |
| Line 11 | 549.2 (10.1) | 7.4 (0.4) | 55.0 (1.7) |
| Line 6 | 564.1 (14.2) | 7.7 (0.3) | 51.3 (3.4) |

Wood density, wood microfibril angle and cell wall crystallinity of of five-month old wild-type and AtGolS3 transgenic poplar trees.

TABLE 8a
Average (standard error) cell wall structural chemistry of galactinol
synthase overexpressing transgenic poplar compared to wild-type poplar.

| | | | | | | | Total |
|---|---|---|---|---|---|---|---|
| | Arabinose | Rhamnose | Galactose | Glucose | Xylose | Mannose | Lignin |
| | (μg/mg) | (μg/mg) | (μg/mg) | (μg/mg) | (μg/mg) | (μg/mg) | (%) |
| Wild-type | 3.5 | 3.7 | 8.0 | 426.3 | 145.9 | 27.4 | 24.8 |
| | (0.4) | (0.1) | (0.4) | (16.4) | (2.2) | (1.5) | (0.1) |
| Line 3 | 3.5 | 3.7 | 9.6 | 461.4 | 133.0 | 29.5 | 23.1 |
| | (0.2) | (0.0) | (0.6) | (8.7) | (4.2) | (0.4) | (0.7) |
| Line 8 | 3.6 | 2.8 | 9.6 | 459.6 | 144.9 | 29.3 | 23.6 |
| | (0.5) | (0.8) | 0.8) | (21.4) | (5.5) | (1.3) | (0.2) |
| Line 11 | 7.1 | 2.7 | 14.0 | 444.4 | 109.1 | 16.5 | 19.4 |
| | (1.1) | (1.2) | (0.8) | (29.9) | (6.9) | (2.0) | (0.3) |
| Line 6 | 6.5 | 3.0 | 16.5 | 502.4 | 110.7 | 15.3 | 18.2 |
| | (0.5) | (0.2) | (2.5) | (31.2) | (4.8) | (0.7) | (0.2) |
| Line 10 | 3.9 | 3.0 | 8.5 | 440.0 | 149.9 | 31.4 | |
| | (1.5) | (1.6) | (0.6) | (31.2) | (8.2) | (5.2) | |
| Line 2 | 3.7 | 2.9 | 11.3 | 452.2 | 155.6 | 34.2 | |
| | (1.6) | (2.3) | (0.7) | (17.8) | (9.0) | (9.4) | |
| Line 5 | 3.1 | 3.9 | 10.6 | 491.1 | 154.7 | 21.7 | |
| | (0.2) | (0.9) | (2.9) | (38.9) | (5.5) | (3.3) | |
| Line 9 | 4.0 | 2.4 | 9.4 | 431.7 | 156.0 | 35.7 | |
| | (1.5) | (1.8) | (2.0) | (22.3) | (4.0) | (2.3) | |

TABLE 8b
Average (standard error) cell wall structural chemistry of galactinol
synthase overexpressing transgenic poplar compared to wild-type poplar.

| | Arabinose | | Rhamnose | | Galactose | | Glucose | | Xylose | | Mannose | | Lignin | |
|---|---|---|---|---|---|---|---|---|---|---|---|---|---|---|
| Lines | μg/mg | | μg/mg | | μg/mg | | μg/mg | | μg/mg | | μg/mg | | % | |
| wt | 3.12 | (0.48) | 6.63 | (0.52) | 8.65 | (0.67) | 466.16 | (6.82) | 161.37 | (4.58) | 24.64 | (0.94) | 24.79 | (0.21) |
| line 3 | 3.00 | (0.40) | 6.37 | (1.79) | 10.80 | (1.16) | 509.43 | (10.59) | 151.57 | (5.62) | 29.02 | (0.84) | 23.06 | (1.27) |
| line 6 | 7.52 | (0.71) | 4.63 | (1.03) | 18.61 | (2.79) | 540.62 | (23.77) | 127.97 | (4.87) | 8.73 | (0.80) | 18.23 | (0.40) |
| line 8 | 3.12 | (0.66) | 5.28 | (0.12) | 10.06 | (1.04) | 481.09 | (9.80) | 151.54 | (5.66) | 22.54 | (0.81) | 23.58 | (0.38) |
| line 11 | 8.67 | (1.30) | 7.15 | (1.35) | 17.02 | (0.97) | 509.45 | (4.27) | 134.61 | (5.25) | 11.39 | (1.44) | 19.38 | (0.47) |

All citations are hereby incorporated by reference.

The present invention has been described with regard to one or more embodiments. However, it will be apparent to persons skilled in the art that a number of variations and modifications can be made without departing from the scope of the

What is claimed is:

1. A method for enhancing cell wall property in a perennial plant or portion of the perennial plant comprising
(a) introducing into the perennial plant or portion of the perennial plant, at least one nucleotide construct comprising a nucleic acid molecule operatively linked to a regulatory region active in the perennial plant, wherein said nucleic acid molecule encodes a polypeptide with galactinol synthase (GolS)-like activity;
(b) growing the perennial plant or portion of the perennial pant under conditions that permit the expression of the nucleic acid, thereby enhancing the cell wall property of the perennial plant or portion of the perennial plant.

2. The method of claim 1, wherein the enhancing cell wall property comprises increased cell wall density, decreased microfibril angle, increased wood density, altered cellulose crystallinity, increased tension wood formation, increased cellulose content, reduced lignin content, altered lignin monomer composition, modified hemicellulose matrix, modified pectin matrix or a combination thereof.

3. The method of claim 1 wherein after the step of growing, the perennial plant comprising the enhanced cell wall property is grown.

4. A method for enhancing cell wall property in a perennial plant or portion of the perennial plant, comprising
(a) providing the perennial plant or a portion of the perennial plant, comprising at least one nucleotide construct comprising a nucleic acid molecule operatively linked to a regulatory region active in the plant, wherein said nucleic acid molecule encodes a polypeptide with galactinol synthase (GolS)-like activity;
(b) growing the perennial plant, or portion of the perennial plant under conditions that permit the expression of the nucleic acid, thereby enhancing the cell wall property of the perennial plant, or portion of the perennial plant.

5. A method for altering level of carbohydrates in a perennial plant or portion of a perennial plant comprising
(a) introducing into the plant or a portion of the plant, at least one nucleotide construct comprising a nucleic acid molecule operatively linked to a regulatory region active in the plant, wherein said nucleic acid molecule encodes a polypeptide with galactinol synthase (GolS)-like activity;
(b) growing the plant or portion of the pant under conditions that permit the expression of the nucleic acid, thereby altering the level of carbohydrates in the plant or portion of the plant.

6. The method of claim 5, wherein the level of total hexose is increased and the level of pentose is decreased or a combination thereof, when compared to the same parameter determined in the plant or portion of the plant of the same species, grown under the same conditions and wherein the plant or portion of the plant is not transformed with a nucleic acid molecule encodes a polypeptide with galactinol synthase (GolS)-like activity.

7. The method of claim 5, wherein the level of galactose and/or glucose is increased and/or the level of xylose is decreased when compared to the same parameter determined of the plant or portion of the plant of the same species, grown under the same conditions and wherein the plant or portion of the plant is not transformed with a nucleic acid molecule encodes a polypeptide with galactinol synthase (GolS)-like activity.

8. A perennial plant produced by the method of any one of claims 1, 4 and 5.

9. The plant of claim 8, wherein the perennial plant is a tree.

10. A perennial plant, portion of a plant or plant cell comprising a nucleic acid comprising a nucleotide sequence encoding a polypeptide with galactinol synthase (GolS)-like activity operatively linked to a regulatory region active in a plant.

11. The perennial plant, portion of the plant or plant cell of claim 10, wherein the polypeptide that is encoded by any one sequence selected from the group consisting of SEQ ID NOS: 2, 4, 6, 16-25, and 38-45.

12. The method of claim 1, wherein the polypeptide is encoded by any one sequence selected from the group consisting of SEQ ID NOS: 2, 4, 6, 16-25, and 38-45.

13. A feedstock or food stuff comprising the perennial plant, portion of the plant or plant cell of claim 8 or 10.

14. The feedstock of claim 13, for use in solid wood, pulp and paper, dissolving pulp industry, biofuel industry, or a combination thereof.

15. A nucleic acid comprising a nucleotide sequence encoding a polypeptide with galactinol synthase (GolS)-like activity operatively linked to a regulatory region active in a plant, wherein the .the polypeptide is encoded by any one sequence selected from the group consisting of SEQ ID NO 16-25, 38, and 45.

16. A method for producing a feedstock for use in pulp and paper or biofuel production comprising,
(a) providing a perennial plant comprising at least one nucleotide construct comprising a nucleic acid molecule operatively linked to a regulatory region active in the perennial plant, wherein said nucleic acid molecule encodes a polypeptide with galactinol synthase (GolS)-like activity; and
(b) growing the perennial plant under conditions that permit the expression of the nucleic acid, thereby producing the feedstock.
